SEQUENCE LISTING (54 sequences)

SEQ ID NO: 1 - protein, 759 aa, Homo sapiens
GPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVG

SEQ ID NO: 2 - DNA, 2397 bp, Homo sapiens
ggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtc

SEQ ID NO: 3 - protein, 761 aa, Mus musculus
QAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTVG

SEQ ID NO: 4 - DNA, 2283 bp, Mus musculus
caggctggcccgcgggccccctgcgcggccgcctgcacttgcgccggggactcgctggactgcagtgggcgcgggctggcgacgctgccccgggacctgccctcctggacgcgcagcctaaacctgagttataacagactctccgagatcgactctgctgcttttgaggacttgacgaatctgcaggaagtgtacctcaacagcaatgagctgacagccataccatcactgggcgctgcttccataggagttgtctctctctttttgcagcacaacaagatccttagtgtggatgggagccagctgaagtcgtacctgtccttggaagtgctggatctgagttccaacaacatcacggaaattcggagctcctgtttcccgaacggcctgcgtataagggaactcaacttggcgagcaaccgcatcagcatcctggagtctggagcatttgatggtctgtcgcggtcactgctgactctccgtctgagcaaaaacaggatcacccagcttcctgtgaaagcgttcaagctacccaggctgacacaactagacctgaatcggaatcggattcggctgattgaaggcctcacgttccaggggctcgacagcttagaggtgctgaggcttcagaggaacaacatcagcaggctgacggacggggccttctgggggctgtctaagatgcacgtgctgcacctggagtacaacagtctggtggaagtgaacagtggctccctctatggcctcacagccctgcaccagctgcacctcagcaacaactccatctctcgaattcagcgtgatggctggagcttctgccaaaagctgcatgagttgattctgtccttcaacaacctcacgcggctggatgaggagagtctagcggagttgagcagcctcagtatcctgcgcctcagtcacaacgccatcagtcacattgctgaaggcgccttcaagggactcaagagtctgcgggtcttggacctggaccataacgagatctcgggtacaatcgaggataccagtggtgcctttacggggcttgacaacctcagcaagctgactctgtttggaaacaagatcaaatctgtggctaagagagccttctcgggcctggaaagcctggaacacctgaaccttggagagaatgcaatcaggtctgtccagtttgatgcctttgcaaagatgaagaaccttaaagagctctacatcagcagtgagagcttcctgtgtgactgccagctcaagtggctgcccccatggctaatgggtaggatgctgcaggcctttgtgacagccacctgtgcccatccagagtcgctgaagggccagagcattttctcagtgctgccagacagctttgtgtgtgatgactttccaaagccacagatcatcacccagcctgagacgaccatggctgtggtgggcaaggacatccgtttcacatgctccgcagccagcagcagcagctcaccaatgaccttcgcctggaagaaggacaatgaggtcctggccaatgcagacatggagaactttgcccacgtccgtgcacaggacggcgaagtgatggagtataccactatcctgcacctccgtcacgtcacctttgggcacgagggccgctaccagtgtatcatcacaaaccactttggctccacatactcccacaaagccaggctcactgtgaatgtgttgccatcattcactaaaataccccatgacattgccatccggactggcaccacagcccgcctcgagtgtgctgccacgggccaccctaaccctcagattgcctggcagaaggatggaggcaccgatttcccggcagctcgtgagcgacgcatgcatgttatgccagacgatgatgtgttcttcatcactgatgtgaaaatagacgacatgggggtctacagctgcactgcccagaactcggcaggctcggtttcagccaacgctaccctcacagtcttagaaactccatccttggcagtgcctctggaagaccgtgtggtaactgtgggagaaacagtggccttccagtgcaaagcaaccgggagccccacaccacgcatcacctggcttaagggaggtcgcccattgagcctcacagagcgccaccatttcactccaggcaaccagctgctggttgttcagaatgtgatgatagacgatgcagggcggtatacctgtgagatgtctaatcccctgggcactgagcgagcacatagccagctgagcattttacctacccctggctgccggaaggatgggaccaccgtaggc

SEQ ID NO: 5 - protein, 5 aa, Artificial Sequence
  Heavy chain CDR 1
NYAMS

SEQ ID NO: 6 - protein, 17 aa, Artificial Sequence
  Heavy chain CDR 2
VISHGGGSTYYADSVKG

SEQ ID NO: 7 - protein, 18 aa, Artificial Sequence
  Heavy chain CDR 3
VISNCHLGVCYYSNGMDV

SEQ ID NO: 8 - protein, 13 aa, Artificial Sequence
  Light chain CDR 1
SGSSSNIGDNYVS

SEQ ID NO: 9 - protein, 7 aa, Artificial Sequence
  Light chain CDR 2
DNNKRPS

SEQ ID NO: 10 - protein, 10 aa, Artificial Sequence
  Light chain CDR 3
WDGSLSAGRV

SEQ ID NO: 11 - protein, 127 aa, Artificial Sequence
  Heavy chain variable region
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSS

SEQ ID NO: 12 - protein, 111 aa, Artificial Sequence
  Light chain variable region
QLVLTQPPSVSAAPGQRVSISCSGSSSNIGDNYVSWYQQVPGSAPKLLIYDNNKRPSGIPDRFSASKSGTSATLGITGLQTGDEADYYCATWDGSLSAGRVFGGGTKLTVL

SEQ ID NO: 13 - DNA, 15 bp, Artificial Sequence
  Heavy chain CDR 1
aactacgccatgagc

SEQ ID NO: 14 - DNA, 51 bp, Artificial Sequence
  Heavy chain CDR 2
gtgatctctcacggcggaggcagcacctactacgccgattctgtgaagggc

SEQ ID NO: 15 - DNA, 54 bp, Artificial Sequence
  Heavy chain CDR 3
gtgatcagcaactgccacctgggcgtgtgctactacagcaacggcatggatgtg

SEQ ID NO: 16 - DNA, 39 bp, Artificial Sequence
  Light chain CDR 1
agcggcagcagcagcaacatcggcgacaactacgtgtcc

SEQ ID NO: 17 - DNA, 21 bp, Artificial Sequence
  Light chain CDR 2
gacaacaacaagcggcccagc

SEQ ID NO: 18 - DNA, 30 bp, Artificial Sequence
  Light chain CDR 3
tgggatggctccctgagcgccggaagagtt

SEQ ID NO: 19 - DNA, 381 bp, Artificial Sequence
  Heavy chain variable region
gaagttcagctgcttgagtctggcggaggactggttcaacctggcggaagcctgagactgtcttgtgccgccagcggcttcaccttcagcaactacgccatgagctgggtccgacaggcccctggaaaaggccttgaatgggtgtccgtgatctctcacggcggaggcagcacctactacgccgattctgtgaagggcagattcaccatcagccgggacaacagcaagaacaccctgtacctgcagatgaacagcctgagagccgaggacaccgccgtgtactattgcgccagagtgatcagcaactgccacctgggcgtgtgctactacagcaacggcatggatgtgtggggccagggcacactggttaccgttagttct

SEQ ID NO: 20 - DNA, 333 bp, Artificial Sequence
  Light chain variable region
caactggttctgacacagcctccaagcgtgtcagctgcccctggacagagagtgtccatcagctgtagcggcagcagcagcaacatcggcgacaactacgtgtcctggtatcagcaggtcccaggctctgcccctaagctgctgatctacgacaacaacaagcggcccagcggcatccccgatagattttctgccagcaagagcggcaccagcgccacactgggaattacaggactgcagacaggcgacgaggccgactactattgtgccacatgggatggctccctgagcgccggaagagtttttggcggaggcaccaagctgaccgtgctt

SEQ ID NO: 21 - protein, 328 aa, Mus musculus
TTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 107 aa, Mus musculus
RTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 330 aa, Homo sapiens [sequence omitted]

SEQ ID NO: 24 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 107 aa, Homo sapiens [sequence omitted]

SEQ ID NO: 26 - protein, 107 aa, Homo sapiens
RSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 28 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 29 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 332 aa, Artificial Sequence
  Heavy chain constant region
ASAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 107 aa, Artificial Sequence
  Light chain constant region
RTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 245 aa, Artificial Sequence
  Hybrid Fc Heavy region
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - DNA, 990 bp, Homo sapiens
gctagcaccaagggcccatcggtcttccccctggcgccctcctccaagagcacctctgggggcacagcggccctgggctgcctggtcaaggactacttccccgaaccggtgacggtgtcgtggaactcaggggccctgaccagcggcgtgcacaccttcccggctgtcctacagtcctcaggactctactccctcagcagcgtggtgaccgtgccctccagcagcctgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 321 bp, Homo sapiens
agatctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - DNA, 999 bp, Artificial Sequence
  Heavy chain constant region
gctagcgccaaaacaacagccccctccgtataccccctagcccccgtatgcggcgacaccaccggctcctccgtaaccctaggctgcctagtaaaaggctacttccccgaacccgtaaccctaacctggaactccggctccctatcctccggcgtacacaccttccccgccgtactacaatccgacctatacaccctatcctcctccgtaaccgtaacctcctccacctggccctcccaatccatcacctgcaacgtagcccaccccgcctcctccaccaaagtagacaaaaaaatcgagccaaggggccccaccatcaagccttgccctccctgcaagtgccctgcccccaacctgctgggcggcccctccgtgttcatcttccctccaaagatcaaggacgtgctgatgatctccctgagccccatcgtgacctgcgtggtggtggacgtgtccgaggacgaccctgacgtgcagatcagctggttcgtgaacaacgtggaggtgcacaccgcccagacccagacccacagagaggactacaactccaccctgagggtggtgagcgccctgccaatccagcaccaggactggatgtccggcaaggagttcaagtgcaaggtgaacaacaaggacctgccagccccaatcgagaggaccatcagcaagcctaagggctccgtgagggccccccaggtgtacgtgctgccccctcctgaggaggagatgaccaagaagcaggtgaccctgacctgcatggtgaccgacttcatgcctgaggacatctacgtggagtggaccaacaacggcaagaccgagctgaactacaagaacaccgagcctgtgctggacagcgacggcagctacttcatgtacagcaagctgagggtggagaagaagaactgggtggagagaaactcctactcctgctccgtggtgcacgagggcctgcacaaccaccacaccaccaagagcttctccagaacccccgggaagtga

SEQ ID NO: 36 - DNA, 321 bp, Artificial Sequence
  Light chain constant region
cgtacggatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 37 - protein, 457 aa, Artificial Sequence
  Heavy chain
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 38 - protein, 457 aa, Artificial Sequence
  Heavy chain
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 218 aa, Artificial Sequence
  Light chain
QLVLTQPPSVSAAPGQRVSISCSGSSSNIGDNYVSWYQQVPGSAPKLLIYDNNKRPSGIPDRFSASKSGTSATLGITGLQTGDEADYYCATWDGSLSAGRVFGGGTKLTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 40 - protein, 218 aa, Artificial Sequence
  Light chain
QLVLTQPPSVSAAPGQRVSISCSGSSSNIGDNYVSWYQQVPGSAPKLLIYDNNKRPSGIPDRFSASKSGTSATLGITGLQTGDEADYYCATWDGSLSAGRVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 1380 bp, Artificial Sequence
  Heavy chain
gaagttcagctgcttgagtctggcggaggactggttcaacctggcggaagcctgagactgtcttgtgccgccagcggcttcaccttcagcaactacgccatgagctgggtccgacaggcccctggaaaaggccttgaatgggtgtccgtgatctctcacggcggaggcagcacctactacgccgattctgtgaagggcagattcaccatcagccgggacaacagcaagaacaccctgtacctgcagatgaacagcctgagagccgaggacaccgccgtgtactattgcgccagagtgatcagcaactgccacctgggcgtgtgctactacagcaacggcatggatgtgtggggccagggcacactggttaccgttagttctgctagcgccaaaacaacagccccctccgtataccccctagcccccgtatgcggcgacaccaccggctcctccgtaaccctaggctgcctagtaaaaggctacttccccgaacccgtaaccctaacctggaactccggctccctatcctccggcgtacacaccttccccgccgtactacaatccgacctatacaccctatcctcctccgtaaccgtaacctcctccacctggccctcccaatccatcacctgcaacgtagcccaccccgcctcctccaccaaagtagacaaaaaaatcgagccaaggggccccaccatcaagccttgccctccctgcaagtgccctgcccccaacctgctgggcggcccctccgtgttcatcttccctccaaagatcaaggacgtgctgatgatctccctgagccccatcgtgacctgcgtggtggtggacgtgtccgaggacgaccctgacgtgcagatcagctggttcgtgaacaacgtggaggtgcacaccgcccagacccagacccacagagaggactacaactccaccctgagggtggtgagcgccctgccaatccagcaccaggactggatgtccggcaaggagttcaagtgcaaggtgaacaacaaggacctgccagccccaatcgagaggaccatcagcaagcctaagggctccgtgagggccccccaggtgtacgtgctgccccctcctgaggaggagatgaccaagaagcaggtgaccctgacctgcatggtgaccgacttcatgcctgaggacatctacgtggagtggaccaacaacggcaagaccgagctgaactacaagaacaccgagcctgtgctggacagcgacggcagctacttcatgtacagcaagctgagggtggagaagaagaactgggtggagagaaactcctactcctgctccgtggtgcacgagggcctgcacaaccaccacaccaccaagagcttctccagaacccccgggaagtga

SEQ ID NO: 42 - DNA, 1371 bp, Artificial Sequence
  Heavy chain
gaagttcagctgcttgagtctggcggaggactggttcaacctggcggaagcctgagactgtcttgtgccgccagcggcttcaccttcagcaactacgccatgagctgggtccgacaggcccctggaaaaggccttgaatgggtgtccgtgatctctcacggcggaggcagcacctactacgccgattctgtgaagggcagattcaccatcagccgggacaacagcaagaacaccctgtacctgcagatgaacagcctgagagccgaggacaccgccgtgtactattgcgccagagtgatcagcaactgccacctgggcgtgtgctactacagcaacggcatggatgtgtggggccagggcacactggttaccgttagttctgctagcaccaagggcccatcggtcttccccctggcgccctcctccaagagcacctctgggggcacagcggccctgggctgcctggtcaaggactacttccccgaaccggtgacggtgtcgtggaactcaggggccctgaccagcggcgtgcacaccttcccggctgtcctacagtcctcaggactctactccctcagcagcgtggtgaccgtgccctccagcagcctgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - DNA, 654 bp, Artificial Sequence
  Light chain
caactggttctgacacagcctccaagcgtgtcagctgcccctggacagagagtgtccatcagctgtagcggcagcagcagcaacatcggcgacaactacgtgtcctggtatcagcaggtcccaggctctgcccctaagctgctgatctacgacaacaacaagcggcccagcggcatccccgatagattttctgccagcaagagcggcaccagcgccacactgggaattacaggactgcagacaggcgacgaggccgactactattgtgccacatgggatggctccctgagcgccggaagagtttttggcggaggcaccaagctgaccgtgcttcgtacggatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 44 - DNA, 654 bp, Artificial Sequence
  Light chain
caactggttctgacacagcctccaagcgtgtcagctgcccctggacagagagtgtccatcagctgtagcggcagcagcagcaacatcggcgacaactacgtgtcctggtatcagcaggtcccaggctctgcccctaagctgctgatctacgacaacaacaagcggcccagcggcatccccgatagattttctgccagcaagagcggcaccagcgccacactgggaattacaggactgcagacaggcgacgaggccgactactattgtgccacatgggatggctccctgagcgccggaagagtttttggcggaggcaccaagctgaccgtgcttagatctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 45 - DNA, 24 bp, Artificial Sequence
  mouse Lrig-1 forward primer
gacggaattcagtgaggagaacct

SEQ ID NO: 46 - DNA, 24 bp, Artificial Sequence
  mouse Lrig-1 reverse primer
caactggtagtggcagcttgtagg

SEQ ID NO: 47 - DNA, 24 bp, Artificial Sequence
  mouse Lrig-2 forward primer
tcacaaggaacattgtctgaacca

SEQ ID NO: 48 - DNA, 24 bp, Artificial Sequence
  mouse Lrig-2 reverse primer
gcctgatctaacacatcctcctca

SEQ ID NO: 49 - DNA, 24 bp, Artificial Sequence
  mouse Lrig-3 forward primer
cagcaccttgagctgaacagaaac

SEQ ID NO: 50 - DNA, 24 bp, Artificial Sequence
  mouse Lrig-3 reverse primer
ccagcctttggtaatctcggttag

SEQ ID NO: 51 - DNA, 24 bp, Artificial Sequence
  mouse FOXP3 forward primer
ctttcacctatcccacccttatcc

SEQ ID NO: 52 - DNA, 24 bp, Artificial Sequence
  mouse FOXP3 reverse primer
attcatctacggtccacactgctc

SEQ ID NO: 53 - DNA, 20 bp, Artificial Sequence
  ACTG1 forward primer
ggcgtcatggtgggcatggg

SEQ ID NO: 54 - DNA, 20 bp, Artificial Sequence
  ACTG1 reverse primer
atggcgtggggaagggcgta

(19) United States
(12) Patent Application Publication
(10) Pub. No.: US 2024/0301050 A1
(43) Pub. Date: Sep. 12, 2024

(54) NOVEL BINDING MOLECULE AND USE THEREOF

(71) Applicant: Good T Cells, Inc., Seoul (KR)

(72) Inventors: Jung Ho Kim, Seoul (KR); Beom Seok Kim, Seoul (KR)

(21) Appl. No.: 18/263,210

(22) PCT Filed: Jan. 27, 2022
(86) PCT No.: PCT/KR2022/001498

§ 371 Date: Jul. 27, 2023

(30) Foreign Application Priority Data
Jan. 27, 2021 (KR) 10-2021-0011845

(51) Int. Cl. C07K 16/28; A61K 39/00; A61K 47/68; A61P 25/28; C07K 14/725; G01N 33/68

(52) U.S. Cl. CPC C07K 16/28; A61K 47/6849; A61P 25/28; C07K 14/7051; G01N 33/6896; A61K 2039/505; C07K 2317/24; C07K 2319/00; G01N 2800/2821

(57) ABSTRACT

The present invention relates to a novel binding molecule with excellent preventive, ameliorative or therapeutic effects on a brain and nervous system disease and uses thereof. The binding molecule provided in the present invention can effectively prevent or treat brain and nervous system diseases, particularly neurodegenerative diseases or neuroinflammatory diseases.

### TECHNICAL FIELD

The present invention relates to a novel binding molecule with excellent preventive, ameliorative or therapeutic effects on a brain and nervous system disease and uses thereof.

### BACKGROUND ART

In degenerative brain and nervous system diseases such as stroke, dementia, and Alzheimer's disease, deterioration in memory, attention, cognitive ability, emotion regulation, and the like is observed, which results from death of neuronal cells and atrophy of nerve branches. Nerve branch elongation in neuronal cells leads to increased neuroplasticity, and thus plays an important role in memory and learning functions of neural circuitry. Therefore, it is predicted that active ingredients, which promote nerve branch elongation and nerve regeneration in neuronal cells, have the potential to be developed as new therapeutic agents for degenerative brain and nervous system diseases.

As the aging population rapidly increases, the incidence of degenerative brain and nervous system diseases is also on the rise. Despite innovative advances in medicine, prophylactic and therapeutic methods for degenerative brain and nervous system diseases are not yet clearly established, and no drugs have been found which have a decisive effect. Currently, therapeutic agents and treatment methods for degenerative brain and nervous system diseases are being developed; however, they often exhibit side effects and toxicity due to long-term use, and only have an effect of alleviating symptoms rather than treating the disease. Therefore, there is an urgent need to develop a material capable of achieving treatment while having decreased side effects and toxicity.

### Technical Problem

An object of the present invention is to provide a binding molecule that has excellent binding ability to the Lrig-1 protein present on the surface of regulatory T cells (Treg) and is excellent in preventing, ameliorating, or treating a brain and nervous system disease.

Another object of the present invention is to provide a nucleic acid molecule encoding said binding molecule according to the present invention.

Another object of the present invention is to provide an expression vector into which the nucleic acid molecule according to the present invention is inserted.

Another object of the present invention is to provide a host cell line transfected with the expression vector according to the present invention.

Another object of the present invention is to provide an antibody-drug conjugate according to the present invention.

Another object of the present invention is to provide a composition for preventing, ameliorating or treating a brain and nervous system disease comprising the binding molecule according to the present invention.

Another object of the present invention is to provide a pharmaceutical composition for preventing or treating a brain and nervous system disease comprising the antibody-drug conjugate according to the present invention as an active ingredient.

Another object of the present invention is to provide a pharmaceutical composition for preventing or treating a brain and nervous system disease comprising the chimeric antigen receptor (CAR) according to the present invention as an active ingredient.

Another object of the present invention is to provide a diagnostic composition and kit for diagnosing a brain and nervous system disease comprising the binding molecule according to the present invention, and a method for providing information for diagnosing a brain and nervous system disease.

Another object of the present invention is to provide a method for preventing or treating a brain and nervous system disease characterized by the binding molecule, antibody or fragment thereof, antibody-drug conjugate (ADC), or chimeric antigen receptor (CAR) according to the present invention.

However, the technical problem to be achieved by the present invention is not limited to the above-mentioned problems, and other problems that are not mentioned will be clearly understood by a skilled person in the art from the following description.

### Solution to Problem

According to an embodiment of the present invention, there is provided a binding molecule that specifically binds to leucine-rich and immunoglobulin-like domains 1 (Lrig-1) protein present on the surface of regulatory T cells (Treg cells).

As used herein, the term “binding molecule” refers to a variable domain comprising an intact immunoglobulin that includes a monoclonal antibody, such as a chimeric, humanized, or human monoclonal antibody, or an immunoglobulin that binds to an antigen, for example, an immunoglobulin fragment that competes with intact immunoglobulins for binding to monomeric HA or trimeric HA of influenza A virus. Regardless of the structure, an antigen-binding fragment binds to the same antigen recognized by intact immunoglobulins. The antigen-binding fragment may include a peptide or polypeptide which contains, out of the amino acid sequence of the binding molecule, an amino acid sequence of two or more contiguous residues, 20 or more contiguous amino acid residues, 25 or more contiguous amino acid residues, 30 or more contiguous amino acid residues, 35 or more contiguous amino acid residues, 40 or more contiguous amino acid residues, 50 or more contiguous amino acid residues, 60 or more contiguous amino acid residues, 70 or more contiguous amino acid residues, 80 or more contiguous amino acid residues, 90 or more contiguous amino acid residues, 100 or more contiguous amino acid residues, 125 or more contiguous amino acid residues, 150 or more contiguous amino acid residues, 175 or more contiguous amino acid residues, 200 or more contiguous amino acid residues, or 250 or more contiguous amino acid residues. The term “antigen-binding fragment”, in particular, includes Fab, F(ab′), F(ab′)2, Fv, dAb, Fd, complementarity determining region (CDR) fragments, single-chain antibodies (scFvs), bivalent single-chain antibodies, single-chain phage antibodies, unibodies, diabodies, triabodies, tetrabodies, polypeptides containing one or more fragments of immunoglobulin which is sufficient for a particular antigen to bind to the polypeptide, and the like. The fragment may be produced synthetically or by enzymatic or chemical digestion of a complete immunoglobulin, or may be produced by genetic engineering methods using recombinant DNA techniques. Production methods are well known in the art.

In the present invention, the “Lrig-1 protein” is a transmembrane protein consisting of 1091 amino acids present on the surface of regulatory T cells, and is composed of leucine-rich repeats (LRRs) and three immunoglobulin-like domains on the extracellular or lumen side, a cell transmembrane sequence, and a cytoplasmic tail portion. The LRIG gene family includes LRIG1, LRIG2, and LRIG3, and the amino acids therebetween are highly conserved. The LRIG1 gene is highly expressed in normal skin and can be expressed in basal and hair follicle cells to regulate proliferation of epithelial stem cells. Therefore, the LRIG1 gene plays an important role in maintaining homeostasis of the epidermis, and its absence may develop psoriasis or skin cancer. It has been reported that in a case where chromosome 3p14.3 portion in which LRIG1 is located is cut off, there is a possibility of developing into cancer cells. In fact, it was identified that expression of LRIG1 is greatly decreased in renal cell carcinoma and cutaneous squamous cell carcinoma. Recently, it has been also found that Lrig-1 is expressed in only about 20 to 30% of cancers. On the other hand, for the purpose of the present invention, the Lrig-1 protein may be, but is not limited to, a protein present in humans or mice.

In the present invention, the Lrig-1 protein may be, but is not limited to, a human-derived polypeptide represented by SEQ ID NO: 1 or a mouse-derived polypeptide represented by SEQ ID NO: 3.

In addition, in the present invention, the Lrig-1 protein represented by SEQ ID NO: 1 may be encoded by a polynucleotide represented by SEQ ID NO: 2, but is not limited thereto.

In addition, in the present invention, the Lrig-1 protein represented by SEQ ID NO: 3 may be encoded by a polynucleotide represented by SEQ ID NO: 4, but is not limited thereto.

In the present invention, the binding molecule may be a binding molecule, comprising:
a heavy chain variable region that contains a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7; and
a light chain variable region that contains a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

In the present invention, the binding molecule may be a binding molecule, comprising a heavy chain variable region consisting of the amino acid sequence represented by SEQ ID NO: 11; and
a light chain variable region consisting of the amino acid sequence represented by SEQ ID NO: 12.

In the present invention, the binding molecule may be a binding molecule, comprising:
a heavy chain variable region that contains a heavy chain CDR1 encoded by the nucleotide sequence represented by SEQ ID NO: 13, a heavy chain CDR2 encoded by the nucleotide sequence represented by SEQ ID NO: 14, and a heavy chain CDR3 encoded by the nucleotide sequence represented by SEQ ID NO: 15; and
a light chain variable region that contains a light chain CDR1 encoded by the nucleotide sequence represented by SEQ ID NO: 16, a light chain CDR2 encoded by the nucleotide sequence represented by SEQ ID NO: 17, and a light chain CDR3 encoded by the nucleotide sequence represented by SEQ ID NO: 18.

In the present invention, the binding molecule may be binding molecule, comprising:
a heavy chain variable region encoded by the nucleotide sequence represented by SEQ ID NO: 19; and
a light chain variable region encoded by the nucleotide sequence represented by SEQ ID NO: 20.

In the present invention, the binding molecule may further comprise a fragment crystallization (Fc) region or a constant region. Here, the Fc region may be an Fc region of an IgA, IgD, IgE, IgM, IgG1, IgG2, IgG3, or IgG4 antibody, or may be derived therefrom. Alternatively, the Fc region may be a hybrid Fc region.

In the present invention, the Fc region may be an Fc region of a mammalian-derived IgA, IgD, IgE, IgM, IgG1, IgG2, IgG3, or IgG4 antibody, and may preferably be an Fc region of a human-derived IgA, IgD, IgE, IgM, IgG1, IgG2, IgG3, or IgG4 antibody. However, the Fc region is not limited thereto.

As an example of the present invention, the constant region may be a mouse-derived IgG2a constant region represented by SEQ ID NO: 21, but is not limited thereto.

As an example of the present invention, the constant region may be a mouse-derived immunoglobulin kappa constant region represented by SEQ ID NO: 22, but is not limited thereto.

As an example of the present invention, the constant region may be a human-derived IgG1 constant region represented by SEQ ID NO: 23 or 24, but is not limited thereto.

As an example of the present invention, the constant region may be a human-derived immunoglobulin kappa constant region represented by SEQ ID NO: 25 or 26, but is not limited thereto.

As an example of the present invention, the constant region may be a human-derived IgG2 constant region represented by SEQ ID NO: 27, but is not limited thereto.

As an example of the present invention, the constant region may be a human-derived IgG3 constant region represented by SEQ ID NO: 28, but is not limited thereto.

As an example of the present invention, the constant region may be a human-derived IgG4 constant region represented by SEQ ID NO: 29, but is not limited thereto.

As an example of the present invention, the constant region may be a constant region represented by SEQ ID NO: 30, but is not limited thereto.

As an example of the present invention, the constant region may be a constant region represented by SEQ ID NO: 31, but is not limited thereto.

As an example of the present invention, the Fc region may be a human-derived immunoglobulin lambda constant region, but is not limited thereto.

In the present invention, the “hybrid Fc” may be derived from a combination of human IgG subclasses or a combination of human IgD and IgG. In a case where the hybrid Fc binds to a biologically active molecule, polypeptide, or the like, the hybrid Fc has effects of not only increasing a serum half-life of the biologically active molecule, but also increasing an expression level of the polypeptide when a nucleotide sequence encoding the Fc-polypeptide fusion protein is expressed.

As an example of the present invention, the hybrid Fc region may be hybrid Fc represented by SEQ ID NO: 32, but is not limited thereto.

In the binding molecule of the present invention, the Fc or constant region may be linked, via a linker, to the variable region. Here, the linker may be linked to the C-terminus of the Fc or constant region, and the N-terminus of the binding molecule of the present invention may be linked to the linker. However, the present invention is not limited thereto.

In the present invention, the “linker” may contain a sequence that can be cleaved by an enzyme that is overexpressed in a tissue or cell having a target disease. In a case where the linker may be cleaved by the overexpressed enzyme as described above, it is possible to effectively prevent activity of a polypeptide from decreasing due to the Fc or constant region. In the present invention, an example of the linker may be preferably a peptide linker consisting of 33 amino acids located in the 282^{nd} to 314th portion of human albumin which is most abundantly present in the blood, and more preferably a peptide linker consisting of 13 amino acids located in the 292^{nd} to 304^{th} portion of the human albumin. Such portions are portions which are mostly exposed to the outside in three-dimensional structure, and thus have a minimum possibility of inducing an immune response in the body. However, the linker is not limited thereto.

The binding molecule of the present invention may further comprise a heavy chain constant region consisting of an amino acid sequence selected from the group consisting of SEQ ID NOs: 21, 23, 24, 27, 28, 29, 30, and 32.

The binding molecule of the present invention may further comprise a light chain constant region consisting of the amino acid sequence represented by SEQ ID NO: 22, 25, 26, and 31.

The binding molecule of the present invention may further comprise:
a heavy chain constant region consisting of the amino acid sequence represented by SEQ ID NO: 21; and
a light chain constant region consisting of the amino acid sequence represented by SEQ ID NO: 22.

The binding molecule of the present invention may further comprise:
a heavy chain constant region represented by an amino acid sequence represented by SEQ ID NO: 23, 24, 27, 28, 29, and 30; and
a light chain constant region represented by an amino acid sequence represented by SEQ ID NO: 25, 26, and 31.

The binding molecule of the present invention may further comprise:
a heavy chain constant region consisting of the amino acid sequence represented by SEQ ID NO: 24; and
a light chain constant region consisting of the amino acid sequence represented by SEQ ID NO: 26.

The binding molecule of the present invention may further comprise:
a heavy chain constant region consisting of the amino acid sequence represented by SEQ ID NO: 30; and
a light chain constant region consisting of the amino acid sequence represented by SEQ ID NO: 31.

The binding molecule of the present invention may further comprise:
a heavy chain constant region consisting of the amino acid sequence represented by SEQ ID NO: 32.

The binding molecule of the present invention may further comprise:
a heavy chain constant region encoded by the nucleotide sequence represented by SEQ ID NO: 33 or 35; and
a light chain constant region encoded by the nucleotide sequence represented by SEQ ID NO: 34 or 36.

The binding molecule of the present invention may be a binding molecule comprising a heavy chain consisting of the amino acid sequence represented by SEQ ID NO: 37 or 38 and a light chain consisting of the amino acid sequence represented by SEQ ID NO: 39 or 40.

The binding molecule of the present invention may be a binding molecule comprising a heavy chain consisting of the amino acid sequence represented by SEQ ID NO: 37 and a light chain consisting of the amino acid sequence represented by SEQ ID NO: 39.

The binding molecule of the present invention may be a binding molecule comprising a heavy chain consisting of the amino acid sequence represented by SEQ ID NO: 38 and a light chain consisting of the amino acid sequence represented by SEQ ID NO: 40.

The binding molecule of the present invention may be a binding molecule comprising a heavy chain encoded by the nucleotide sequence represented by SEQ ID NO: 41 or 43 and a light chain encoded by the nucleotide sequence represented by SEQ ID NO: 42 or 44.

The binding molecule of the present invention may be a binding molecule comprising a heavy chain encoded by the nucleotide sequence represented by SEQ ID NO: 41 and a light chain encoded by the nucleotide sequence represented by SEQ ID NO: 42.

The binding molecule of the present invention may be a binding molecule comprising a heavy chain encoded by the nucleotide sequence represented by SEQ ID NO: 43 and a light chain encoded by the nucleotide sequence represented by SEQ ID NO: 44.

The binding molecule of the present invention is characterized by being an antibody or a fragment thereof, but is not limited thereto. The antibody includes all of a monoclonal antibody, a full-length antibody, or an antibody fragment which is a portion of an antibody, has the ability to bind to Lrig-1 protein, and can compete with the binding molecule of the present invention in binding to an epitope on Lrig-1.

As used herein, the term “antibody” refers to a protein molecule which serves as a receptor that specifically recognizes an antigen, including an immunoglobulin molecule that is immunologically reactive with a particular antigen. For the purpose of the present invention, the antigen may be Lrig-1 protein present on the surface of regulatory T cells. Preferably, the antibody may specifically recognize the leucine-rich region or immunoglobulin-like domain of the Lrig-1 protein, but is not limited thereto.

In the present invention, the “immunoglobulin” has a heavy chain and a light chain, and each of the heavy chain and the light chain comprises a constant region and a variable region. The variable region of each of the light chain and the heavy chain contains three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

In addition, as used herein, the term “monoclonal antibody” refers to an antibody molecule of a single molecular composition which is obtained from substantially the same antibody population, and exhibits single binding specificity and affinity for a particular epitope.

In the present invention, the “full-length antibody” has a structure with two full-length light chains and two full-length heavy chains in which each light chain is linked to a heavy chain by a disulfide bond, and includes IgA, IgD, IgE, IgM, and IgG. The IgG includes, as subtypes thereof, IgG1, IgG2, IgG3, and IgG4.

In addition, as used herein, the term “antigen fragment” refers to a fragment that retains an antigen-binding function, and includes Fab, Fab′, F(ab′)2, Fv, and the like. The Fab has a structure with variable regions of light and heavy chains, a constant region of the light chain, and a first constant region (CH1 domain) of the heavy chain, and has one antigen-binding site. In addition, Fab′ is different from Fab in that Fab′ has a hinge region containing at least one cysteine residue at the C-terminus of the heavy chain CH1 domain. F(ab′)₂ antibodies are produced with cysteine residues at the hinge region of Fab′ forming a disulfide bond. Fv (variable fragment) refers to the smallest antibody fragment having only a heavy chain variable region and a light chain variable region. Double-chain Fv (dsFv) is configured to be such that a heavy chain variable region and a light chain variable region are linked to each other by a disulfide bond, and single-chain Fv (scFv) is configured to be such that a heavy chain variable region and a light chain variable region are covalently linked to each other, in general, via a peptide linker. The antibody fragment may be obtained as Fab or F(ab′)₂ fragment in a case where a proteolytic enzyme, for example, papain or pepsin is used, and may be produced through a genetic recombinant technique.

In addition, in the present invention, the antibody may be, but is not limited to, a chimeric antibody, a humanized antibody, a bivalent, bispecific molecule, a minibody, a domain antibody, a bispecific antibody, an antibody mimetic, a unibody, a diabody, a triabody, or a tetrabody, or a fragment thereof. In the present invention, the “chimeric antibody” is an antibody which is obtained by recombination of a variable region of a mouse antibody and a constant region of a human antibody, and has a greatly improved immune response as compared with the mouse antibody.

In addition, as used herein, the term “humanized antibody” refers to an antibody obtained by modifying a protein sequence of an antibody derived from a non-human species so that the protein sequence is similar to an antibody variant naturally produced in humans. For example, the humanized antibody may be prepared as follows. Mouse-derived CDRs may be recombined with a human antibody-derived FR to prepare a humanized variable region, and the humanized variable region may be recombined with a constant region of a preferred human antibody to prepare a humanized antibody.

In the present invention, the binding molecule may be provided as a bispecific antibody or a bispecific antigen-binding fragment which is capable of binding to Lrig-1 protein and also binding to another protein.

In the present invention, the bispecific antibody and the bispecific antigen-binding fragment may comprise the binding molecule according to the present invention. As an example of the present invention, the bispecific antibody and the bispecific antigen-binding fragment comprise an antigen-binding domain capable of binding to Lrig-1 protein, wherein the antigen-binding domain capable of binding to Lrig-1 protein may comprise or consist of the binding molecule according to the present invention.

The bispecific antibody and the bispecific antigen-binding fragment provided in the present invention comprise an antigen-binding domain, which is a binding molecule capable of binding to Lrig-1 protein according to the present invention, and an antigen-binding domain capable of binding to another target protein. Here, the antigen-binding domain capable of binding another target protein may be an antigen-binding domain capable of binding to a protein other than Lrig-1 protein, for example, but not limited to, PD-1 or a cell surface receptor. However, the antigen-binding domain is not limited thereto.

The bispecific antibody and the bispecific antigen-binding fragment according to the present invention may be provided in any suitable format. For example, the bispecific antibody or the bispecific antigen-binding fragment may be a bispecific antibody conjugate (for example, IgG2, F(ab′)2, or CovX-body), a bispecific IgG or IgG-like molecule (for example, IgG, scFv4-Ig, IgG-scFv, scFv-IgG, DVD-Ig, IgG-sVD, sVD-IgG, or 2 in 1-IgG, mAb2, or Tandemab common LC), an asymmetric bispecific IgG or IgG-like molecule (for example, kih IgG, kih IgG common LC, CrossMab, kih IgG-scFab, mAb-Fv, charge pair, or SEED-body), a small bispecific antibody molecule (for example, diabody (Db), dsDb, DART, scDb, tandAb, tandem scFv (taFv), tandem dAb/VHH, triple body, triple head, Fab-scFv, or F(ab′)2-scFv2), a bispecific Fc and CH3 fusion protein (for example, taFv-Fc, di-diabody, scDb-CH3, scFv-Fc-scFv, HCAb-VHH, scFv-kih-Fc, or scFv-kih-CH3), or a bispecific fusion protein (for example, scFv2-albumin, scDb-albumin, taFv-toxin, DNL-Fab3, DNL-Fab4-IgG, DNL-Fab4-IgG-cytokine 2). The bispecific antibody and the bispecific antigen-binding fragment according to the invention may be designed and prepared by those skilled in the art.

A method for producing the bispecific antibody in the present invention comprises forming a reducing disulfide or non-reducing thioether bond, and chemical crosslinking of an antibody or antibody fragment. For example, N-succinimidyl-3-(-2-pyridyldithio)-propionate (SPDP) may be used, for example, for chemically crosslinking a Fab fragment through an SH-group at the hinge region, to generate a disulfide-linked bispecific F(ab)2 heterodimer.

In addition, an alternative method for producing the bispecific antibody in the present invention comprises fusing an antibody-producing hybridoma with, for example, polyethylene glycol, to produce quadroma cells capable of secreting bispecific antibodies.

The bispecific antibody and the bispecific antigen-binding fragment according to the invention may also be, for example, recombinantly produced by expression from a nucleic acid construct that encodes a polypeptide for an antigen-binding molecule.

For example, a DNA construct that contains a sequence encoding light and heavy chain variable domains for two antigen-binding domains (that is, light and heavy chain variable domains for an antigen-binding domain capable of binding to PD-1, and light and heavy chain variable domains for an antigen-binding domain capable of binding to another target protein), and a sequence encoding a suitable linker or dimerization domain between the antigen-binding domains may be prepared by molecular cloning techniques. Subsequently, a recombinant bispecific antibody may be produced by expression of the construct (for example, in vitro) in a suitable host cell (for example, a mammalian host cell), and then the expressed recombinant bispecific antibody may be optionally purified.

Antibodies may be produced by an affinity maturation process in which a modified antibody with improved affinity for an antigen as compared with an unmodified parent antibody is produced. An affinity matured antibody may be produced by a procedure known in the art.

In addition, the binding molecule provided in the present invention may include a variant of the amino acid sequence as long as the variant can specifically bind to Lrig-1 protein. For example, in order to improve binding affinity and/or other biological properties of an antibody, modifications may be made to an amino acid sequence of the antibody. Such modifications include, for example, deletions, insertions, and/or substitutions of amino acid sequence residues of the antibody.

Such amino acid variations are made based on relative similarity of amino acid side chain substituents such as hydrophobicity, hydrophilicity, charge, and size. According to analysis on sizes, shapes, and types of amino acid side chain substituents, it can be seen that arginine, lysine, and histidine are all positively charged residues; alanine, glycine, and serine have similar sizes; and phenylalanine, tryptophan, and tyrosine have similar shapes. Thus, based on these considerations, it can be said that arginine, lysine, and histidine; alanine, glycine, and serine; and phenylalanine, tryptophan, and tyrosine are biologically functional equivalents.

In introducing variations, the hydropathic index of amino acids may be considered. Each amino acid has been assigned hydropathic index depending on its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The hydropathic amino acid index is very important in conferring the interactive biological function on a protein. It is known that substitution with an amino acid having similar hydropathic index allows a protein to retain similar biological activity. In a case where variations are introduced with reference to the hydropathic index, substitutions are made between amino acids that exhibit a hydropathic index difference of preferably within ±2, more preferably within ±1, and even more preferably within ±0.5.

Meanwhile, it is also well known that substitutions between amino acids having similar hydrophilicity values result in proteins with equivalent biological activity. As disclosed in U.S. Pat. No. 4,554,101, respective amino acid residues have been assigned the following hydrophilicity values: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In a case where variations are introduced with reference to the hydrophilicity values, substitutions may be made between amino acids that exhibit a hydrophilicity value difference of preferably within ±2, more preferably within ±1, and even more preferably within ±0.5.

Amino acid exchanges in proteins which do not entirely alter activity of a molecule are known in the art. The most commonly occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Gln/Glu.

Given the above-described variations with biologically equivalent activity, it is interpreted that the binding molecule of the present invention also includes sequences that exhibit substantial identity with the sequences listed in the Sequence Listing.

As used herein, the term “substantial identity” refers to a sequence showing at least 61% homology, more preferably 70% homology, even more preferably 80% homology, and most preferably 90% homology when the sequence of the present invention is aligned with any other sequence so that they maximally correspond to each other, and the aligned sequence is analyzed by using an algorithm typically used in the art. Alignment methods for comparison of sequences are known in the art. NCBI Basic Local Alignment Search Tool (BLAST) is accessible from the National Center for Biological Information (NBCI), or the like, and may be used in conjunction with sequencing programs, such as blastp, blasm, blastx, tblastn, and tblastx, on the internet. BLAST is accessible at http://www.ncbi.nlm.nih.gov/BLAST/. Sequence homology comparison methods using this program can be identified online (http://www.ncbi.nlm.nih.gov/BLAST/blast_help.html).

In the present invention, the binding molecule, preferably the antibody, may be produced by a conventional method for producing an antibody, and may be produced by affinity maturation.

As used herein, the term “affinity maturation” refers to a process in which antibodies having increased affinity for an antigen are produced by activated B cells in the course of an immune response. For the purpose of the present invention, the affinity maturation allows antibodies or antibody fragments to be produced due to affinity maturation based on the principles of mutation and selection, in the same process that occurs in nature.

The binding molecule, preferably the antibody, provided in the present invention can effectively prevent, ameliorate, or treat brain and nervous system diseases, in particular, neurodegenerative diseases or neuroinflammatory diseases.

According to another embodiment of the present invention, there is provided a nucleic acid molecule encoding the binding molecule provided in the present invention.

The nucleic acid molecule of the present invention includes all nucleic acid molecules obtained by translating the amino acid sequences of the binding molecules provided in the present invention to polynucleotide sequences, as known to those skilled in the art. Therefore, various polynucleotide sequences may be prepared by an open reading frame (ORF), and all of these polynucleotide sequences are also included in the nucleic acid molecule of the present invention.

The nucleic acid molecule of the present invention may comprise a heavy chain encoded by the nucleotide sequence represented by SEQ ID NO: 41 or 43 and a light chain encoded by the nucleotide sequence represented by SEQ ID NO: 42 or 44.

The nucleic acid molecule of the present invention may comprise a heavy chain encoded by the nucleotide sequence represented by SEQ ID NO: 41 and a light chain encoded by the nucleotide sequence represented by SEQ ID NO: 42.

The nucleic acid molecule of the present invention may comprise a heavy chain encoded by the nucleotide sequence represented by SEQ ID NO: 43 and a light chain encoded by the nucleotide sequence represented by SEQ ID NO: 44.

According to another embodiment of the present invention, there is provided an expression vector into which the isolated nucleic acid molecule provided in the present invention is inserted.

In the present invention, the “vector” is a nucleic acid molecule capable of transporting another nucleic acid linked thereto. One type of vector is a “plasmid,” which refers to circular double-stranded DNA into which an additional DNA segment can be ligated. Another type of vector is a phage vector. Yet another type of vector is a viral vector, where an additional DNA segment can be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form of vector.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Qpp, T-even, T2, T3, T7; plant viruses. Any expression vector known, to those skilled in the art, as expression vectors can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

In order to facilitate purification of the nucleic acid molecule of the present invention, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

According to another embodiment of the present invention, there is provided a host cell line transfected with the expression vector provided in the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells. However, the host cell is not limited thereto, and any cell known to those skilled in the art which can be used as a host cell line is available.

According to another embodiment of the present invention, there is provided an antibody-drug conjugate (ADC) comprising the antibody provided in the present invention and a drug.

As used herein, the term “antibody-drug conjugate (ADC)” refers to a form in which the drug and the antibody are chemically linked to each other without degrading biological activity of the antibody and the drug. In the present invention, the antibody-drug conjugate denotes a form in which the drug is bound to an amino acid residue at the N-terminus of the heavy and/or light chain of the antibody, specifically, a form in which the drug is bound to an α-amine group at the N-terminus of the heavy and/or light chain of the antibody.

In the present invention, examples of the reactive group capable of reacting and crosslinking with the α-amine group are not particularly limited in terms of type as long as the reactive group can react and crosslink with an α-amine group at the N-terminus of a heavy or light chain of an antibody. The reactive group includes all types of groups known in the art which react with an amine group. The reactive group may, for example, be any one of isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester, but is not limited thereto.

As used herein, the term “drug” may mean any substance having a certain biological activity for a cell, which is a concept including DNA, RNA, or a peptide. The drug may be in a form which contains a reactive group capable of reacting and crosslinking with an α-amine group, and also includes a form which contains a reactive group capable of reacting and crosslinking with an α-amine group and to which a linker is linked.

According to another embodiment of the present invention, there is provided a pharmaceutical composition for preventing or treating a brain and nervous system disease comprising, as an active ingredient, a chimeric antigen receptor (CAR) comprising an antigen-specific binding domain, a linking domain and a CD3 zeta (ζ) signaling domain.

As used herein, the term “chimeric antigen receptor” or “CAR” refers to an engineered receptor comprising an extracellular antigen binding domain and an intracellular signaling domain. Although the most common type of CAR comprises a single chain variable fragment (scFv) derived from a monoclonal antibody fused to the transmembrane and intracellular domains of a T cell co-receptor, such as the CD3 zeta (ζ) chain, the present invention is not limited to these domains. Rather, the “chimeric antigen receptor” or “CAR” as used herein refers to any receptor engineered to express any intracellular signaling molecule and any extracellular antigen binding domain fused or linked thereto. In the present invention, the binding domain may comprise a single chain variable fragment (scFv) capable of specifically recognizing the Lrig-1 protein. In the present invention, the term “single chain variable fragment” or “scFv” refers to a fusion protein of a variable heavy chain (VH) and a variable light chain (VL) of an antibody by a peptide linker between VL and VH.

In addition, in the present invention, the VH domain and the VL domain may be connected through a flexible linker. In the present invention, the flexible linker may be a glycine/serine linker of about 10 to 30 amino acids (e.g., 30, 25, 20, 19, 18, 17, 16, 15, 14, 13, 12, 11, 10, 9, 8, 7, 6, or 5 amino acids), preferably 15 amino acids in length. In the present invention, the linker length can act as an important determining site for chimeric antigen receptors, so a linker shorter than the above range can increase affinity, but can also cause intracellular multimer formation to impair CAR expression, while linkers longer than this range may reduce antigen affinity by moving the VL and VH CDRs farther apart in proximity.

The chimeric antigen receptor of the present invention may further comprise at least one of a hinge region (or spacer) and a signaling domain. In the present invention, the hinge region is a region connecting antigen-binding domain and the transmembrane domain, and is also called a “spacer,” and has the purpose of extending the antigen-binding domain from the T cell membrane or the NK cell membrane. In the present invention, the hinge region can be obtained from any suitable sequence from any genus, including, for example, human or parts thereof, or may include CD8, CD28, 4-1BB, OX40, all or part of the CD3 zeta (ζ) chain, T cell receptor α or β chain, CD28, CD3c, CD45, CD4, CD5, CD8, CD9, CD16, CD22, CD33, CD37, CD64, CD80, CD86, CD134, CD137, ICOS, CD154, functional derivatives thereof, or combinations thereof commonly used in the art, but is not limited thereto. In addition, the hinge region may comprise one selected from immunoglobulins (e.g., IgG1, IgG2, IgG3, IgG4, and IgD) without being limited to immunoglobulins, but is not limited thereto.

In the present invention, the signaling domain refers to a part of a chimeric antigen receptor found or engineered to be found inside a T cell. The signaling domain may or may not comprise a transmembrane that serves to fix the chimeric antigen receptor in the plasma membrane of T cells. The transmembrane domain and the signaling domain may be derived from the same protein (e.g., CD3 zeta (ζ) molecule), or the transmembrane domain and the signaling domain may be derived from different proteins (e.g., transmembrane domain of CD28 and intracellular signaling domain of CD3 zeta (ζ) molecule, or vice versa).

In the present invention, the transmembrane domain comprises, for example, T cell receptor α or β chain, all or part of the CD3 zeta (ζ) chain, CD28, CD38, CD45, CD4, CD5, CD8, CD9, CD16, CD22, CD33, CD37, CD64, CD80, CD86, CD134, CD137, ICOS, CD154, functional derivatives thereof, or combinations thereof, but is not limited thereto. The costimulatory domain may comprise 4-1BB (CD137), OX40, CD27, CD28, CD30, CD40, PD-1, CD2, CD7, CD258, natural killer group 2 member C (NKG2C), natural killer group 2 member (NKG2D), B7-H3, CD83, ICAM-1, ligands that bind to LFA-1 (CD11a/CD18) or ICOS, active fragments thereof, functional derivatives thereof, or a functional signaling domain derived from a polypeptide comprising a combination thereof, but is not limited thereto.

In the present invention, the signaling domain may comprise a functional signaling domain derived from polypeptides comprising all or part of CD3 zeta (ζ), common FcR gamma (FcER1G), FcgammaRIIIa, FcRbeta (Fc epsilon lip), CD3gamma, CD3delta, CD3 epsilon, CD79a, CD79b, DNAX-activating protein 10 (DAP10), DNAX-activating protein 12 (DAP12), an active fragment thereof, a functional derivative thereof, or a combination thereof, but is not limited thereto, and such signaling domains are known in the art.

According to one embodiment of the present invention, in the chimeric antigen receptor (CAR) comprising an antigen-specific binding domain, a linking domain and a CD3 zeta (ζ) signaling domain, there is provided a chimeric antigen receptor wherein the specific binding domain is a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

In the present invention, the drug may include any drug regardless of type as long as the drug can treat brain and nervous system diseases, in particular, neurodegenerative diseases or neuroinflammatory diseases.

According to another embodiment of the present invention, the present invention relates to a composition for preventing, ameliorating, or treating a brain and nervous system disease comprising the binding molecule, nucleic acid molecule, expression vector, host cell line, or antibody-drug conjugate (ADC) of the present invention as an active ingredient.

The brain and nervous system disease to be prevented, ameliorated, or treated by the composition provided in the present invention may be a neurodegenerative disease or neuroinflammatory disease.

In the present invention, the “neurodegenerative disease” may refer to a disease caused by decreased function or loss of neurons, and the “neuroinflammatory disease” may refer to a disease caused by excessive inflammatory responses in the nervous system. As a specific example, the neurodegenerative disease or neuroinflammatory disease in the present invention may be selected from, but is not limited to, the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

In addition, the composition provided in the present invention may be used as a pharmaceutical composition or a food composition. However, the present invention is not limited thereto.

In the present invention, the “prevention” may include, without limitation, any act of blocking symptoms caused by a neurodegenerative disease or neuroinflammatory disease, or suppressing or delaying the symptoms, using the composition of the present invention.

In the present invention, the “treatment” or “amelioration” may include, without limitation, any act capable of ameliorating or beneficially altering symptoms caused by a neurodegenerative disease or neuroinflammatory disease, using the composition of the present invention.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition of the present invention may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition according to the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, frequency of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way. The pharmaceutical composition according to the present invention may be formulated in the form of pills, sugar-coated tablets, capsules, liquids, gels, syrups, slurries, or suspensions.

The food composition that includes the composition of the present invention may be prepared in the form of various foods, for example, beverages, gums, tea, vitamin complexes, powders, granules, tablets, capsules, confections, rice cakes, bread, and the like. The food composition of the present invention is composed of a plant extract having little toxicity and side effects, and thus can be used without worries in a case of being ingested for a long time for preventive purposes.

When the composition of the present invention is included in the food composition, it may be added in an amount corresponding to a rate of 0.10% to 50% of the total weight.

Here, in a case where the food composition is prepared in the form of beverages, there is no particular limitation except that the beverage contains the food composition at an indicated proportion, and the beverage may contain various flavoring agents, natural carbohydrates, or the like as additional ingredients, similarly to conventional beverages. That is, examples of the natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like).

In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like.

These ingredients may be used individually or in combination. A proportion of such additives is not so important, and is generally selected from the range of 0.1 to about 50 parts by weight per 100 parts by weight of the composition of the present invention.

According to still yet another embodiment of the present invention, there is provided a method for preventing, ameliorating, or treating a brain and nervous system disease, comprising a step of administering, to an individual in need thereof, the binding molecule provided in the present invention; a nucleic acid molecule encoding the binding molecule; an expression vector into which the nucleic acid molecule is inserted; a host cell line transfected with the expression vector; or the antibody-drug conjugate (ADC) provided in the present invention.

In the present invention, the “individual” is an individual suspected of developing a brain and nervous system disease, and the individual suspected of developing a brain and nervous system disease means a mammal, such as mice and domestic animals, including humans, who has developed or is likely to develop the disease in question. However, any individual, who is treatable with the active substance provided in the present invention, is included therein without limitation.

The brain and nervous system disease treated by the method of the present invention may be a neurodegenerative disease or neuroinflammatory disease. As a specific example, the neurodegenerative disease or neuroinflammatory disease in the present invention may be selected from, but is not limited to, the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

The method of the present invention may comprise a step of administering, in a pharmaceutically effective amount, the binding molecule provided in the present invention; a nucleic acid molecule encoding the binding molecule; an expression vector into which the nucleic acid molecule is inserted; a host cell line transfected with the expression vector; or the antibody-drug conjugate (ADC) provided in the present invention.

An appropriate total daily amount used may be determined by an attending physician or veterinarian within the scope of sound medical judgment, and administration may be made once or several times. However, for the purposes of the present invention, a specific therapeutically effective amount for a particular patient is preferably applied differently depending on various factors, including type and degree of reaction to be achieved, the specific composition comprising the active ingredient, including whether other agents are used therewith as the case may be, the patient's age, body weight, general health status, sex, and diet, frequency of administration, route of administration, secretion rate of the composition comprising the active ingredient, duration of treatment, and drugs used simultaneously or in combination with the specific composition, and similar factors well known in the medical field.

Meanwhile, the method for preventing or treating a brain and nervous system disease may be, but is not limited to, a combination therapy that further comprises a step of administering a compound or substance having therapeutic activity against one or more diseases.

In the present invention, the “combination” should be understood to represent simultaneous, individual, or sequential administration. In a case where the administration is made in a sequential or individual manner, the second component should be administered at intervals such that beneficial effects of the combination are not lost.

In the present invention, the dosage of the binding molecule or the antibody-drug conjugate may be, but is not limited to, about 0.0001 μg to 500 mg per kg of patient's body weight.

According to another embodiment of the present invention, there is provided a diagnostic composition for an immune-related disease comprising an agent for measuring the expression level of the Lrig-1 protein or its extracellular domain or a gene encoding the Lrig-1 protein present on the surface of a T cell using the binding molecule of the present invention.

The T cells of the present invention may be regulatory T cells.

The diagnostic composition of the present invention may comprise, for example, an agent that measures the expression level of the Lrig-1 protein or its extracellular domain or a gene encoding the Lrig-1 protein present on the surface of an activated regulatory T cell, that is, regulatory T cells with activated suppression of effector T cells, but is not limited thereto.

The diagnostic composition of the present invention may further comprise a protein, more than one selected from the group consisting of CD25, TIGIT, LAG3, CTLA-4, GITR, OX40, ICOS, PD-1, TIM-3, CCR4, FR4, CD15s, PI-16, and Lrig-1 proteins present on the surface of T cells, and an agent for measuring the expression level of a gene encoding the same. As such, when the expression level of various proteins or genes encoding such is further measured on the surface of T cells, a significant synergistic effect can be achieved in diagnosing the target disease compared to measuring the Lrig-1 protein or its extracellular domain or gene encoding it alone.

Although the agent measuring the expression level of the Lrig-1 protein or its extracellular domains of the present invention, and the expression level of more than one protein selected from the group consisting of CD25, TIGIT, LAG3, CTLA-4, GITR, OX40, ICOS, PD-1, TIM-3, CCR4, FR4, CD15s, PI-16, and Lrig-1 protein are not particularly limited, it may comprise, for example, at least one selected from the group consisting of an antibody, oligopeptide, ligand, peptide nucleic acid (PNA), and aptamer that specifically binds to the protein.

The agent measuring the expression level of the gene encoding the Lrig-1 protein of the present invention (in other words, LAIR1) or the gene encoding the extracellular domain of the Lrig-1 protein, and a gene encoding more than one protein selected from the group consisting of CD25, TIGIT, LAG3, CTLA-4, GITR, OX40, ICOS, PD-1, TIM-3, CCR4, FR4, CD15s, PI-16, and Lrig-1 may comprise at least one selected from the group consisting of a primer, probe, LNA, and antisense nucleotides that specifically bind to the gene.

As used herein, the term “primer” refers to a fragment that recognizes a target gene sequence, and consists of a forward and reverse primer pair, but preferably provides analysis results with specificity and sensitivity. High specificity can be imparted when the primers amplify the target gene sequence containing complementary primary binding sites while not causing non-specific amplification because the nucleic acid sequence of the primer is inconsistent with the non-target sequence present in the sample.

As used herein, the term “probe” refers to a substance that can specifically bind to a target substance to be detected in a sample, and a substance that can specifically confirm the presence of a target substance in a sample through binding. Given probes are commonly used in the art, the type of probe is not limited, but preferably can be peptide nucleic acid (PNA), locked nucleic acid (LNA), peptide, polypeptide, protein, RNA, or DNA, with PNA being the most preferred. More specifically, the probe is a biomaterial that includes those derived from or similar to those from living organisms or those manufactured in vitro, for example, enzymes, proteins, antibodies, microorganisms, animal and plant cells and organs, nerve cells, DNA, and RNA. DNA may comprise cDNA, genomic DNA and oligonucleotides, while RNA may comprise genomic RNA, mRNA, oligonucleotides, while proteins may comprise antibodies, antigens, enzymes, peptides, and the like.

According to another embodiment of the present invention, there is provided a diagnostic composition for a brain and nervous system disease, comprising the binding molecule provided in the present invention.

In the present invention, the kit may be, but is not limited to, an RT-PCR kit, a DNA chip kit, an ELISA kit, a protein chip kit, a rapid kit, or a multiple reaction monitoring (MRM) kit.

The diagnostic kit of the present invention may further include one or more other component compositions, solutions, or devices suitable for assay methods. For example, the diagnostic kit of the present invention may further include essential elements required for performing reverse transcription polymerase reaction. The kit for reverse transcription polymerase reaction includes a pair of primers specific for a gene encoding a marker protein. The primer pair is a nucleotide having a sequence specific to a nucleic acid sequence of the gene, and may have a length of about 7 bp to 50 bp, or about 10 bp to 30 bp. The kit may also include a primer pair specific for a nucleic acid sequence of a control gene. In addition to those mentioned above, the kit for reverse transcription polymerase reaction may include test tubes or other suitable containers, reaction buffers (with varying pH and magnesium concentrations), deoxynucleotides (dNTPs), enzymes such as Taq-polymerase and reverse transcriptase, DNase and RNase inhibitors, DEPC-treated water, sterilized water, and the like.

In addition, the diagnostic kit of the present invention may include essential elements required for performing a DNA chip assay. The DNA chip kit may include a substrate to which a cDNA or oligonucleotide corresponding to a gene or a fragment thereof is attached; reagents, agents, and enzymes for preparing a fluorescence-labeled probe; and the like. The substrate may also contain a cDNA or oligonucleotide corresponding to a control gene or a fragment thereof.

In addition, the diagnostic kit of the present invention may include essential elements required for performing ELISA. The ELISA kit includes an antibody specific for the marker protein. The antibodies are antibodies with high specificity and affinity for the marker protein and little cross-reactivity with other proteins, and include monoclonal antibodies, polyclonal antibodies, or recombinant antibodies. The ELISA kit may also include an antibody specific for a control protein. In addition to those mentioned above, the ELISA kit may include reagents capable of detecting bound antibodies, for example, labeled secondary antibodies, chromophores, enzymes (which are, for example, conjugated with an antibody) and substrates thereof, and other substances capable of binding to antibodies.

According to another embodiment of the present invention, the present invention relates to a method for providing information on diagnosis of an immune-related disease comprising a step of measuring the expression level of Lrig-1 protein or extracellular domain thereof present on the surface of T cells in a biological sample isolated from a target subject using the binding molecule of the present invention, or the expression level of a gene encoding such.

The T cells of the present invention may be regulatory T cells.

The step of measuring the expression level of the present invention may be to further measure the expression levels of a protein, more than one selected from the group consisting of CD25, TIGIT, LAG3, CTLA-4, GITR, OX40, ICOS, PD-1, TIM-3, CCR4, FR4, CD15s, PI-16, and Lrig-1 proteins present on the surface of T cells, or the expression levels of a gene encoding the same. As such, when the expression level of various proteins or genes encoding such is further measured on the surface of T cells, a significant synergistic effect can be achieved in diagnosing the target disease compared to measuring the Lrig-1 protein or the gene encoding it alone.

In the present invention, the “target individual” refers to an individual for whom it is uncertain whether the disease has developed and who has a high probability of developing the disease.

In the present invention, the “biological sample” may refer to any material, biological fluid, tissue or cell obtained or derived from an individual, and can comprise, for example, whole blood, leukocytes, peripheral blood mononuclear cells, buffy coat, plasma, serum, sputum, tears, mucus, nasal washes, nasal aspirates, breath, urine, semen, saliva, peritoneal washings, pelvic fluids, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extracts, or cerebrospinal fluid, but is not limited thereto.

The Lrig-1 protein or extracellular domain thereof of the present invention may be expressed on the cell surface of T cells, in particular, regulatory T cells, for example, activated regulatory T cells, that is the cell surface of activated regulatory T cells with activated suppressive ability of effector T cells.

In the present invention, examples of the method for measuring or comparatively analyzing the expression level of the Lrig-1 protein, extracellular domain thereof, and more than one protein selected from the group consisting of CD25, TIGIT, LAG3, CTLA-4, GITR, OX40, ICOS, PD-1, TIM-3, CCR4, FR4, CD15s, PI-16 and Lrig-1 include, but are not limited to, protein chip assay, immunoassay, ligand binding assay, matrix assisted laser desorption/ionization time of flight mass spectrometry (MALDI-TOF), sulface enhanced laser desorption/ionization time of flight mass spectrometry (SELDI-TOF), radioimmunoassay, radial immunodiffusion, ouchterlony immunodiffusion, rocket immunoelectrophoresis, immunohistochemical staining, complement fixation assay, two-dimensional electrophoresis, liquid chromatography-mass spectrometry (LC-MS), liquid chromatography-mass spectrometry/mass spectrometry (LC-MS/MS), Western blotting, and enzyme linked immunosorbent assay (ELISA).

The analysis method for measuring the expression level of a gene in order to confirm the presence and expression level of genes encoding the Lrig-1 protein or its extracellular domain, and more than one protein selected from the group consisting of CD25, TIGIT, LAG3, CTLA-4, GITR, OX40, ICOS, PD-1, TIM-3, CCR4, FR4, CD15s, PI-16 and Lrig-1 may be, but is not limited to, reverse transcription polymerase reaction (RT-PCR), competitive RT-PCR, real-time RT-PCR, RNase protection assay (RPA), Northern blotting or DNA chip.

In the present invention, the method may comprise a step of predicting that the target individual has a high probability of developing an immune-related disease in a case where the expression level of Lrig-1 protein or its extracellular domain, or the gene encoding it measured in the biological sample of the target subject is decreased compared to the normal control.

In the present invention, the method may comprise a step of predicting that the target individual has a high probability of developing an immune-related disease in a case where the expression level of more than one protein selected from the group consisting of CD25, TIGIT, LAG3, CTLA-4, GITR, OX40, ICOS, PD-1, TIM-3, CCR4, FR4, CD15s, PI-16 and Lrig-1 proteins, or where the expression level of the genes encoding such proteins is changed (increased or decreased) compared to the normal control.

In the present invention, the method may comprise a step of predicting that the target individual has a high probability of developing an immune-related disease in a case where the expression level of Lrig-1 protein or the gene encoding it is decreased compared to a normal control.

Furthermore, in the case of predicting or diagnosing that an immune-related disease is highly likely to occur by measuring the expression level of Lrig-1 protein or its extracellular domain or the gene encoding it, the method may further comprise a step of administering a drug for the disease to the individual.

According to one embodiment of the present invention, there is provided a binding molecule comprising a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

According to one embodiment of the present invention, there is provided a binding molecule comprising a heavy chain variable region comprising a heavy chain CDR1 encoded by the nucleotide sequence represented by SEQ ID NO: 13, a heavy chain CDR2 encoded by the nucleotide sequence represented by SEQ ID NO: 14, and a heavy chain CDR3 encoded by the nucleotide sequence represented by SEQ ID NO: 15, and a light chain variable region comprising a light chain CDR1 encoded by the nucleotide sequence represented by SEQ ID NO: 16, a light chain CDR2 encoded by the nucleotide sequence represented by SEQ ID NO: 17, and a light chain CDR3 encoded by the nucleotide sequence represented by SEQ ID NO: 18.

According to one embodiment of the present invention, there is provided a binding molecule comprising a heavy chain variable region consisting of the amino acid sequence represented by SEQ ID NO: 11, and a light chain variable region consisting of the amino acid sequence represented by SEQ ID NO: 12.

According to one embodiment of the present invention, there is provided a binding molecule comprising a heavy chain variable region encoded by the nucleotide sequence represented by SEQ ID NO: 19, and a light chain variable region encoded by the nucleotide sequence represented by SEQ ID NO: 20.

According to one embodiment of the present invention, there is provided a binding molecule that further comprises a fragment crystallization region (Fc region) or a constant region.

According to one embodiment of the present invention, there is provided a binding molecule wherein the Fc region is an Fc region of an IgA, IgD, IgE, IgM, IgG1, IgG2, IgG3 or IgG4, or a hybrid Fc.

According to one embodiment of the present invention, there is provided a binding molecule that further comprises a heavy chain constant region consisting of an amino acid sequence selected from the group consisting of SEQ ID NO: 21, 23, 24, 27, 28, 29, 30 and 32.

According to one embodiment of the present invention, there is provided a binding molecule that further comprises a light chain constant region consisting of an amino acid sequence selected from the group consisting of SEQ ID NO: 22, 25, 26, and 31.

According to one embodiment of the present invention, there is provided a binding molecule that further comprises a heavy chain constant region encoded by the nucleotide sequence represented by SEQ ID NO: 33 or 35, and a light chain constant region encoded by the nucleotide sequence represented by SEQ ID NO: 34 or 36.

According to one embodiment of the present invention, there is provided a binding molecule that comprises a heavy chain consisting of the amino acid sequence represented by SEQ ID NO: 37 or 38, and a light chain consisting of the amino acid sequence represented by SEQ ID NO: 39 or 40.

According to one embodiment of the present invention, there is provided a binding molecule that comprises a heavy chain consisting of the amino acid sequence represented by SEQ ID NO: 37, and a light chain consisting of the amino acid sequence represented by SEQ ID NO: 39.

According to one embodiment of the present invention, there is provided a binding molecule that comprises a heavy chain consisting of the amino acid sequence represented by SEQ ID NO: 38 and a light chain consisting of the amino acid sequence represented by SEQ ID NO: 40.

According to one embodiment of the present invention, there is provided a binding molecule that comprises a heavy chain encoded by the nucleotide sequence represented by SEQ ID NO: 41 or 43, and a light chain encoded by the nucleotide sequence represented by SEQ ID NO: 42 or 44.

According to one embodiment of the present invention, there is provided a binding molecule that comprises a heavy chain encoded by the nucleotide sequence represented by SEQ ID NO: 41, and a light chain encoded by the nucleotide sequence represented by SEQ ID NO: 42.

According to one embodiment of the present invention, there is provided a binding molecule that comprises a heavy chain encoded by the nucleotide sequence represented by SEQ ID NO: 43 and a light chain encoded by the nucleotide sequence represented by SEQ ID NO: 44.

According to one embodiment of the present invention, the binding molecule provides a binding molecule that is an antibody or a fragment thereof.

According to one embodiment of the present invention, there is provided a binding fragment wherein the antibody is a chimeric antibody, a humanized antibody, a bivalent, bispecific molecule, a minibody, a domain antibody, a bispecific antibody, an antibody mimic, an unibody, a diabody, a triabody, a tetrabody, or fragments thereof.

According to one embodiment of the present invention, a nucleic acid molecule encoding the binding molecule is provided.

According to one embodiment of the present invention, an expression vector into which the nucleic acid molecule is inserted is provided.

According to one embodiment of the present invention, a host cell line transfected with the expression vector is provided.

According to one embodiment of the present invention, there is provided an antibody-drug conjugate (ADC) that comprises an antibody that comprises a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10, and a drug.

According to one embodiment of the present invention, there is provided a pharmaceutical composition for preventing or treating a brain and nervous system disease comprising the binding molecule of the present invention as an active ingredient.

According to one embodiment of the present invention, there is provided a pharmaceutical composition wherein the brain and nervous system disease is a neurodegenerative disease or a neuroinflammatory disease.

According to one embodiment of the present invention, there is provided a pharmaceutical composition wherein the neurodegenerative disease or neuroinflammatory disease is at least one selected from the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

According to one embodiment of the present invention, in a chimeric antigen receptor (CAR) comprising an antigen-specific binding domain, a linking domain and a CD3 zeta (ζ) signaling domain, there is provided a chimeric antigen receptor wherein the specific binding domain is a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

According to one embodiment of the present invention, there is provided a pharmaceutical composition for preventing or treating a brain and nervous system disease comprising the chimeric antigen receptor as an active ingredient.

According to one embodiment of the present invention, there is provided a diagnostic composition for a brain and nervous system disease comprising the binding molecule of the present invention.

According to one embodiment of the present invention, there is provided a diagnostic composition for a brain and nervous system disease comprising the composition of the present invention.

According to one embodiment of the present invention, there is provided a method for providing information for diagnosing a brain and nervous system disease comprising the step of measuring the expression level of Lrig-1 protein on the surface of T cells in a biological sample isolated from a target subject using the binding molecule of the present invention.

According to another embodiment of the present invention, there is provided a method for preventing or treating a brain and nervous system disease characterized by a binding molecule comprising a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

According to one embodiment of the present invention, the binding molecule is an antibody or a fragment thereof, and there is provided a method for preventing or treating a brain and nervous system disease wherein the antibody is characteristically a chimeric antibody, a humanized antibody, a bivalent, a bispecific molecule, a minibody, a domain antibody, a bispecific antibody, an antibody mimic, an unibody, a diabody, a triabody, a tetrabody, or fragments thereof.

According to another embodiment of the present invention, there is provided a method for preventing or treating a brain and nervous system disease characterized by an antibody-drug conjugate comprising an antibody that comprises a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising alight chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10, and a drug.

According to one embodiment of the present invention, in a chimeric antigen receptor (CAR) comprising an antigen-specific binding domain, a linking domain and a CD3 zeta (ζ) signaling domain, there is provided a method for preventing or treating a brain and nervous system disease characterized by a chimera antigen receptor, wherein the specific binding domain is a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

### Advantageous Effects of Invention

The binding molecule provided in the present invention can specifically prevent, ameliorate, or treat brain and nervous system diseases such as various neurodegenerative or neuroinflammatory diseases.

### BRIEF DESCRIPTION OF DRAWINGS

FIG. 1 illustrates a structure of the Lrig-1 protein according to an embodiment of the present invention.

FIG. 2 illustrates a structure of the Lrig-1 protein according to an embodiment of the present invention.

FIG. 3 illustrates an expression level of Lrig-1 mRNA according to an embodiment of the present invention.

FIG. 4 illustrates an expression level of Lrig-1 mRNA according to an embodiment of the present invention.

FIG. 5 illustrates an expression level of Lrig-1 mRNA according to an embodiment of the present invention.

FIG. 6 illustrates expression levels of Lrig-1, Lrig-2, and Lrig-3 mRNAs according to an embodiment of the present invention.

FIG. 7 illustrates results obtained by comparing expression levels of Lrig-1 protein in regulatory T cells and non-regulated T cells according to an embodiment of the present invention.

FIG. 8 illustrates expression level of the Lrig-1 protein on the surface of regulatory T cells according to an embodiment of the present invention.

FIG. 9 illustrates an experimental design for identifying therapeutic effects of monoclonal antibodies on Alzheimer's, according to an embodiment of the present invention.

FIG. 10 illustrates a photograph of results obtained with a fluorescence microscope by staining the brain cortex and hippocampal tissue of the mouse with Thioflavin S (ThS) obtained after each treatment of Alzheimer's-induced mice with the monoclonal antibody according to an embodiment of the present invention.

FIG. 11 illustrates a graph showing the results of calculating the ThS+ area ratio (%) relative to normal mice after staining the brain cortex and hippocampal tissue of the mouse with Thioflavin S (ThS) obtained after each treatment of Alzheimer's-induced mice with the monoclonal antibody according to an embodiment of the present invention.

FIG. 12 illustrates a graph showing changes in spontaneous alternation (%) values as a result of Y-maze test after each treatment of Alzheimer's-induced mice with the monoclonal antibody according to an embodiment of the present invention.

FIG. 13 illustrates a graph showing changes in preference values as a result of a novel object recognition experiment after each treatment of Alzheimer's-induced mice with the monoclonal antibody according to an embodiment of the present invention.

FIG. 14 illustrates a graph showing changes in the time taken to reach the position of the platform as a result of the water maze experiment after each treatment of Alzheimer's-induced mice with the monoclonal antibody according to an embodiment of the present invention.

FIG. 15 illustrates a graph showing changes in the number of times the Alzheimer's-induced mice passed the target as a result of the water maze experiment after each treatment of Alzheimer's-induced mice with the monoclonal antibody according to an embodiment of the present invention.

FIG. 16 illustrates a graph showing calculated changes in the time taken to reach the position of the platform during the water maze experiment after each treatment of Alzheimer's-induced mice with the monoclonal antibody according to an embodiment of the present invention.

FIG. 17 illustrates an experimental design of Alzheimer's-induced 5xFAD mice and 6xTg mice for identifying therapeutic effects of monoclonal antibodies on Alzheimer's, according to an embodiment of the present invention.

FIG. 18 illustrates a graph showing changes in spontaneous alternation (%) values as a result of Y-maze test after each treatment of Alzheimer's-induced 5xFAD mice and 6xTg mice with the monoclonal antibody according to an embodiment of the present invention.

FIG. 19 illustrates a graph showing changes in preference values as a result of a novel object recognition experiment after each treatment of Alzheimer's-induced 6xTg mice with the monoclonal antibody according to an embodiment of the present invention.

FIG. 20 illustrate a graph showing changes in retention time as a result of a passive avoidance experiment after each treatment of Alzheimer's-induced 5xFAD mice and 6xTg mice with the monoclonal antibody according to an embodiment of the present invention.

### BEST DESCRIPTION FOR IMPLEMENTING THE INVENTION

According to one embodiment of the present invention, there is provided a binding molecule comprising a heavy chain variable region comprising a heavy chain CDR1 consisting of the amino acid sequence represented by SEQ ID NO: 5, a heavy chain CDR2 consisting of the amino acid sequence represented by SEQ ID NO: 6, and a heavy chain CDR3 consisting of the amino acid sequence represented by SEQ ID NO: 7, and a light chain variable region comprising a light chain CDR1 consisting of the amino acid sequence represented by SEQ ID NO: 8, a light chain CDR2 consisting of the amino acid sequence represented by SEQ ID NO: 9, and a light chain CDR3 consisting of the amino acid sequence represented by SEQ ID NO: 10.

### Detailed Description of Invention

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

### [Preparation Example] T Cell Subset Cell Culture

In order to identify whether the Lrig-1 protein is expressed only in regulatory T cells (Treg), the subsets of T cells, Th0, Th1, Th2, Th17, and iTreg, were prepared. The iTreg refers to cells whose differentiation has been artificially induced in a medium containing the following composition, unlike nTreg which has been naturally isolated.

The subsets of the T cells were induced to differentiate into respective cells by first isolating naive T cells obtained from the spleen of mice, causing RPMI1640 (Invitrogen Gibco, Grand Island, NY) nutrient medium that contains 10% fetal bovine serum (FBS; HyClone, Logan, UT) to further contain the respective ingredients of Table 1 below, and performing 72-hour incubation in an incubator at 37° C., 5% CO2.

TABLE 1

| Differentiated cell | Composition |
|---|---|
| Th0 | anti-CD3, anti-CD28 |
| Th1 | IL-12, anti-IL-4 antibody |
| Th2 | IL-4, anti-IFNβ |
| Th17 | IL-6, TGFβ, anti-IFNβ, anti-IL-4 |
| iTreg | IL-2, TGFβ |

### [Example 1] Structural Analysis of Lrig-1

A three-dimensional steric structure of the extracellular domain of the Lrig-1 protein was predicted to produce antibodies specific for the Lrig-1 protein, a surface protein of regulatory T cells.

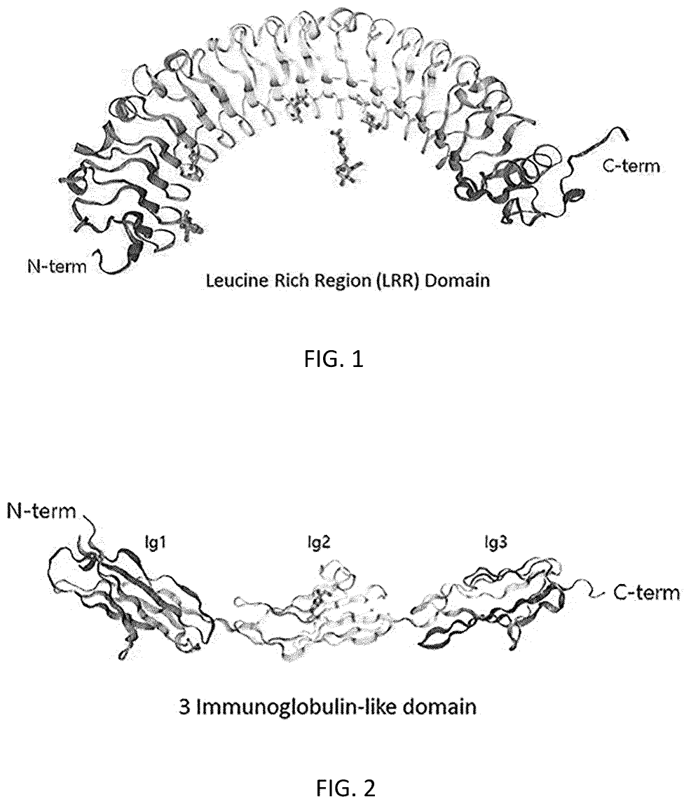

First, in order to predict base sequences of epitopes (epitopes), tools of Uniprot (http://www.uniprot.org) and RCSB Protein Data Bank (http://www.rcsb.org/pdb) were used to predict a three-dimensional steric structure of the extracellular domain (ECD) of the Lrig-1 protein so that the structure of ECD is identified. Then, the results are illustrated in FIGS. 1 and 2.

As illustrated in FIG. 1, a total of 15 leucine-rich regions of LRR1 to LRR15 existed in the Lrig-LRR domain (amino acid sequence at positions 41 to 494) in the extracellular domain of the Lrig-1 protein. Each of the LRR domains is composed of 23 to 27 amino acids, with 3 to 5 leucine being present.

In addition, as illustrated in FIG. 2, three immunoglobulin-like domains exist in amino acid sequences at positions 494 to 781 of the Lrig-1 protein in the extracellular domain of the Lrig-1 protein.

### [Example 2] Prediction of Lrig-1 Epitope Amino Acid Sequence

Prediction of the above base sequence was performed using Ellipro server (http://tools.iedb.org/ellipro/) which is an epitope prediction software based on a structure of the Lrig-1 protein. The Ellipro search engine was used because it corresponds to a search engine known to be the most reliable among the existing algorithms for predicting an epitope.

The extracellular domain analyzed in Example 1 was entered into the epitope prediction software, and a total of 22 contiguous epitope amino acid sequences were predicted, and a total of 8 discontinuous epitope amino acid sequences were predicted.

### [Production Examples 1 and 2] Production of Monoclonal Antibodies Specific to Lrig-1 Protein

Antibodies specific for the Lrig-1 protein according to the present invention were produced. The present antibodies were not produced by specifying a certain epitope, but were produced as antibodies capable of binding to any site on the Lrig-1 protein.

In order to produce the antibodies, cells expressing the Lrig-1 protein were produced. More specifically, a DNA fragment corresponding to SEQ ID NO: 2 and pcDNA (hygro) were cleaved with a cleavage enzyme, incubated at 37° C., and ligated to produce pcDNA into which a DNA sequence of the Lrig-1 protein is inserted. The thus produced pcDNA into which SEQ ID NO: 2 is inserted was introduced, through transfection, into T cells, so that the Lrig-1 protein is allowed to be expressed on the surface of the T cells.

Light and heavy chain amino acid sequences capable of binding to Lrig-1 expressed on the cell surface were selected from the Human scFv library.

The selected heavy and light chain amino acid sequences were fused with the heavy chain constant region represented by SEQ ID NO: 24 or 30 and the light chain constant region represented by SEQ ID NO: 26 or 31, respectively, to produce monoclonal antibodies. The sequences of the monoclonal antibodies are shown in Table 2 below.

TABLE 2

| | | | | Sequence |
|---|---|---|---|---|
| Classification | Clone | Location | Amino acid sequence | information |
| Production | GTC310-01 | Heavy | EVQLLESGGGLVQPGGSLRLSCAASG | SEQ ID NO: |
| Example 1 | mouse | chain | FTFS NYAMS WVRQAPGKGLEWVS | 37 |
| | antibody | | VISHGGGSTYYADSVKG | |
| | | | RFTISRDNSKNTLYLQMNSLRAEDTA | |
| | | | VYYCAR VISNCHLGVCYYSNGMDV | |
| | | | WGQGTLVTVSSAKTTAPSVYPLAPVC | |
| | | | GDTTGSSVTLGCLVKGYFPEPVTLTW | |
| | | | NSGSLSSGVHTFPAVLQSDLYTLSSSV | |
| | | | TVTSSTWPSQSITCNVAHPASSTKVD | |
| | | | KKIEPRGPTIKPCPPCKCPAPNLLGGPS | |
| | | | VFIFPPKIKDVLMISLSPIVTCVVVDVS | |
| | | | EDDPDVQISWFVNNVEVHTAQTQTH | |
| | | | REDYNSTLRVVSALPIQHQDWMSGK | |
| | | | EFKCKVNNKDLPAPIERTISKPKGSVR | |
| | | | APQVYVLPPPEEEMTKKQVTLTCMV | |
| | | | TDFMPEDIYVEWTNNGKTELNYKNT | |
| | | | EPVLDSDGSYFMYSKLRVEKKNWVE | |
| | | | RNSYSCSVVHEGLHNHHTTKSFSRTP | |
| | | | GK | |
| | | Light | QLVLTQPPSVSAAPGQRVSISC | SEQ ID NO: |
| | | chain | SGSSSNIGDNYVS | 39 |
| | | | WYQQVPGSAPKLLIY DNNKRPS | |
| | | | GIPDRFSASKSGTSATLGITGLQTGDE | |
| | | | ADYYCAT WDGSLSAGRV | |
| | | | FGGGTKLTVL | |
| | | | RADAAPTVSIFPPSSEQLTSGGASVVC | |
| | | | FLNNFYPKDINVKWKIDGSERQNGVL | |
| | | | NSWTDQDSKDSTYSMSSTLTLTKDEY | |
| | | | ERHNSYTCEATHKTSTSPIVKSFNRNE | |
| | | | C | |
| Production | GTC310-01 | Heavy | EVQLLESGGGLVQPGGSLRLSCAASG | SEQ ID NO: |
| Example 2 | humanized | chain | FTFS NYAMS WVRQAPGKGLEWVS | 38 |
| | antibody | | VISHGGGSTYYADSVKG | |
| | | | RFTISRDNSKNTLYLQMNSLRAEDTA | |
| | | | VYYCAR VISNCHLGVCYYSNGMDV | |
| | | | WGQGTLVTVSSASTKGPSVFPLAPSS | |
| | | | KSTSGGTAALGCLVKDYFPEPVTVSW | |
| | | | NSGALTSGVHTFPAVLQSSGLYSLSSV | |
| | | | VTVPSSSLGTQTYICNVNHKPSNTKV | |
| | | | DKKVEPKSCDKTHTCPPCPAPELLGG | |
| | | | PSVFLFPPKPKDTLMISRTPEVTCVVV | |
| | | | DVSHEDPEVKFNWYVDGVEVHNAKT | |
| | | | KPREEQYNSTYRVVSVLTVLHQDWL | |
| | | | NGKEYKCKVSNKALPAPIEKTISKAK | |
| | | | GQPREPQVYTLPPSREEMTKNQVSLT | |
| | | | CLVKGFYPSDIAVEWESNGQPENNYK | |
| | | | TTPPVLDSDGSFFLYSKLTVDKSRWQ | |
| | | | QGNVFSCSVMHEALHNHYTQKSLSLS | |
| | | | PGK | |
| | | Light | QLVLTQPPSVSAAPGQRVSISC | SEQ ID NO: |
| | | chain | SGSSSNIGDNYVS | 40 |
| | | | WYQQVPGSAPKLLIY DNNKRPS | |
| | | | GIPDRFSASKSGTSATLGITGLQTGDE | |
| | | | ADYYCAT WDGSLSAGRV | |
| | | | FGGGTKLTVL | |
| | | | RTVAAPSVFIFPPSDEQLKSGTASVVC | |
| | | | LLNNFYPREAKVQWKVDNALQSGNS | |
| | | | QESVTEQDSKDSTYSLSSTLTLSKADY | |
| | | | EKHKVYACEVTHQGLSSPVTKSFNRG | |
| | | | EC | |

### [Example 3] Identification of Specific Expression of Lrig-1 mRNA in Regulatory T Cells

Verification was made of whether the Lrig-1 protein can act as a biomarker specific for regulatory T cells.

For the verification, CD4⁺ T cells were isolated using magnet-activated cell sorting (MACS), through CD4 beads, from the spleen of mice. Subsequently, regulatory T (CD4⁺CD25⁺ T) cells and non-regulatory T (CD4⁺CD25⁺ T) cells were isolated with a fluorescence-activated cell sorter (FACS) using a CD25 antibody. For the respective cells and the cells differentiated in Preparation Example 1, mRNA was extracted using Trizol, and gDNA was removed from genomic RNA using gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively identify an expression level of Lrig-1 mRNA in the cDNA.

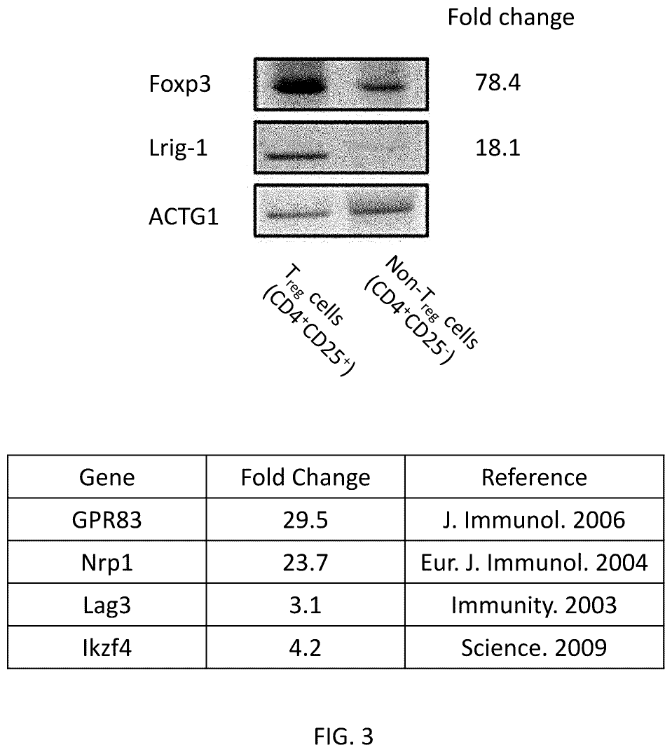

The real-time polymerase chain reaction was performed with primers shown in Table 3 below using SYBR Green (Molecular Probes) by the protocol provided by the manufacturer under conditions of 40 cycles consisting of 95° C. for 3 minutes, 61° C. for 15 seconds, 72° C. for 30 seconds, a relative gene expression level was calculated using the ACT method, and normalized using HPRT. The results are illustrated in FIGS. 3 to 6.

TABLE 3

| | | Sequence |
|---|---|---|
| Primer | Sequence | Information |
| Mouse | Forward 5′-GAC GGA ATT CAG TGA GGA GAA CCT-3′ | SEQ ID NO: 45 |
| Lrig-1 | Reverse 5′-CAA CTG GTA GTG GCA GCT TGT AGG-3′ | SEQ ID NO: 49 |
| Mouse | forward 5′-TCA CAA GGA ACA TTG TCT GAA CCA-3′ | SEQ ID NO: 47 |
| Lrig-2 | reverse 5′-GCC TGA TCT AAC ACA TCC TCC TCA-3′ | SEQ ID NO: 48 |
| Mouse | forward 5′-CAG CAC CTT GAG CTG AAC AGA AAC-3′ | SEQ ID NO: 49 |
| Lrig-3 | reverse 5′-CCA GCC TTT GGT AAT CTC GGT TAG-3′ | SEQ ID NO: 50 |
| Mouse | forward 5′-CTT TCA CCT ATC CCA CCC TTA TCC-3′ | SEQ ID NO: 51 |
| FOXP3 | reverse 5′-ATT CAT CTA CGG TCC ACA CTG CTC-3′ | SEQ ID NO: 52 |
| ACTG1 | forward 5′-GGC GTC ATG GTG GGC ATG GG-3′ | SEQ ID NO: 43 |
| | reverse 5′-ATG GCG TGG GGA AGG GCG TA-3′ | SEQ ID NO: 54 |

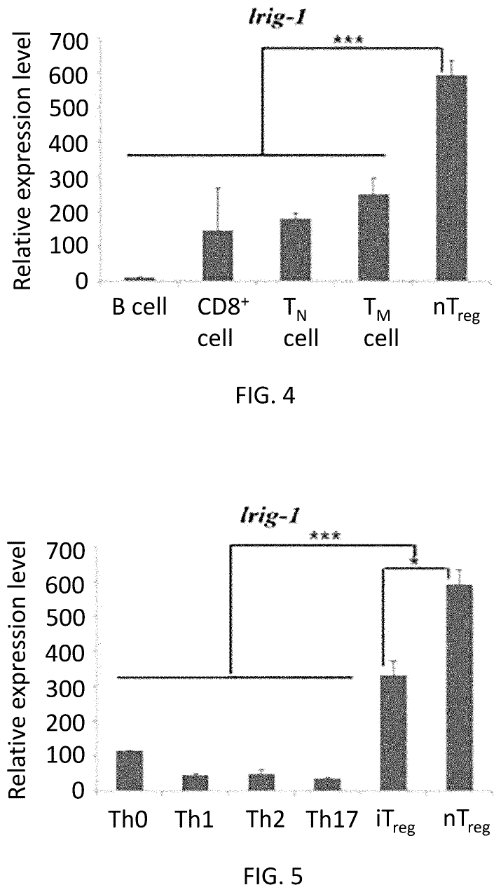

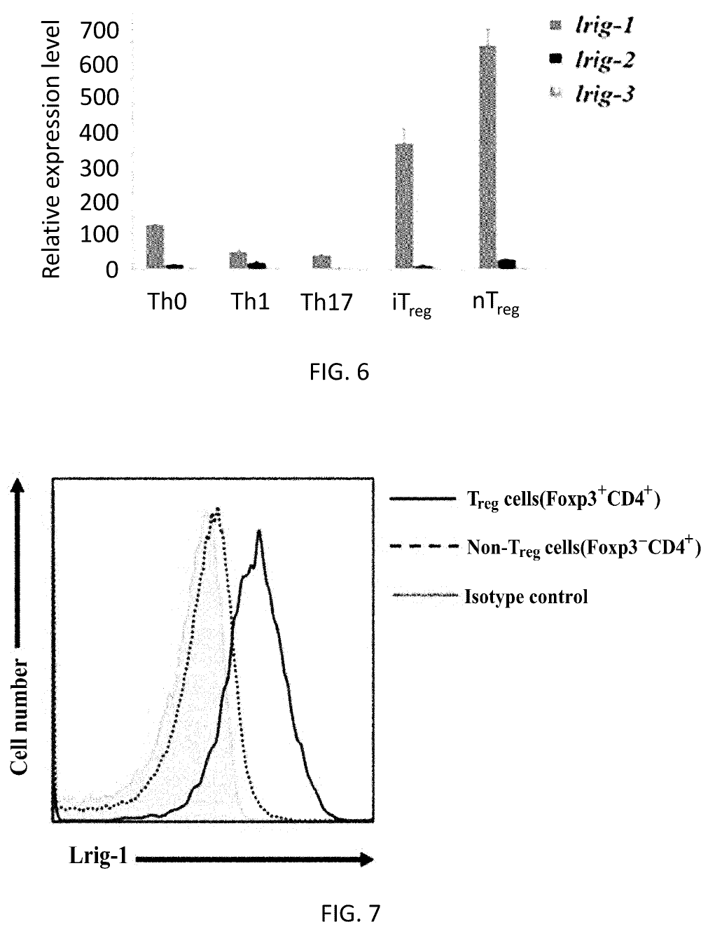

As illustrated in FIG. 3, it can be seen that the expression of Lrig-1 in regulatory T (CD4+CD25+T) cells is 18.1 times higher than non-regulatory T (CD4+CD25⁻ T) cells. This was about 10 times higher expression level than Lag3 and Ikzf4, which are previously known markers for regulatory T cells. In addition, as illustrated in FIGS. 4 and 5, the expression of Lrig-1 mRNA was remarkably high in regulatory T cells as compared with other types of immune cells, and in particular, was remarkably high in naturally isolated regulatory T cells (nTreg) as compared with induced regulatory T cells (iTreg cells). In addition, as illustrated in FIG. 6, expression of Lrig-1 was the highest among Lrig-1, Lrig-2, and Lrig-3 which correspond to the Lrig family.

From the above results, it can be seen that the Lrig-1 protein according to the present invention is specifically expressed in regulatory T cells, in particular, naturally-occurring regulatory T cells.

### [Example 4] Identification of Specific Expression of Lrig-1 Protein in Regulatory T Cells

It was identified whether the Lrig-1 protein expressed from Lrig-1 mRNA is specifically expressed only in regulatory T cells.

Using FOXP3-RFP-knocked-in mice, the FOXP3-RFP obtained by coupling red fluorescence protein (RFP) to FOXP3 promoter, a transcription factor specific for regulatory T cells, CD4⁺ T cells were isolated using magnet-activated cell sorting (MACS), through CD4 beads, from the spleen of the mice. Subsequently, using RFP protein, regulatory T (CD4⁺RFP⁺ T) cells and non-regulatory T (CD4⁺RFP⁺ T) cells were obtained by performing isolation through a fluorescence-activated cell sorter (FACS). The respective cells were stained with the purchased Lrig-1 antibody and a negative control was stained with an isotype-matched control antibody, to measure an expression level of Lrig-1 with the fluorescence-activated cell sorter. The results are illustrated in FIG. 7.

As illustrated in FIG. 7, the non-regulatory T cells indicated by a dotted line showed almost the same expression level of Lrig-1 as the negative control, whereas there were a large number of cells with high expression level of Lrig-1 in the regulatory T cells.

From the above results, it can be seen that the Lrig-1 protein according to the present invention is specifically expressed in regulatory T cells.

### [Example 5] Identification of Specific Expression of Lrig-1 Protein on Surface of Regulatory T Cells

From the viewpoint that in order to be a target of cell therapy, the Lrig-1 protein must be expressed on the surface of regulatory T cells, which in turn allows a more effective target therapy, it was identified whether the Lrig-1 protein is expressed on the surface of the regulatory T cells.

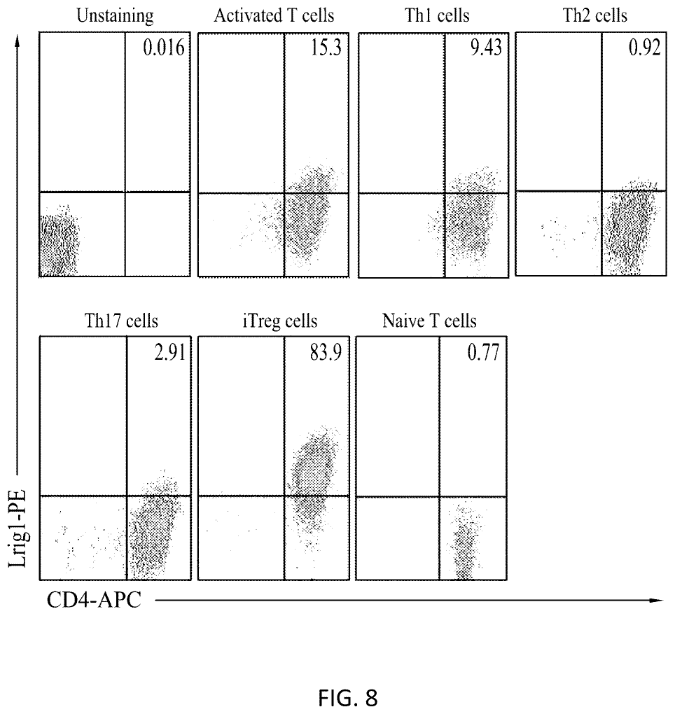

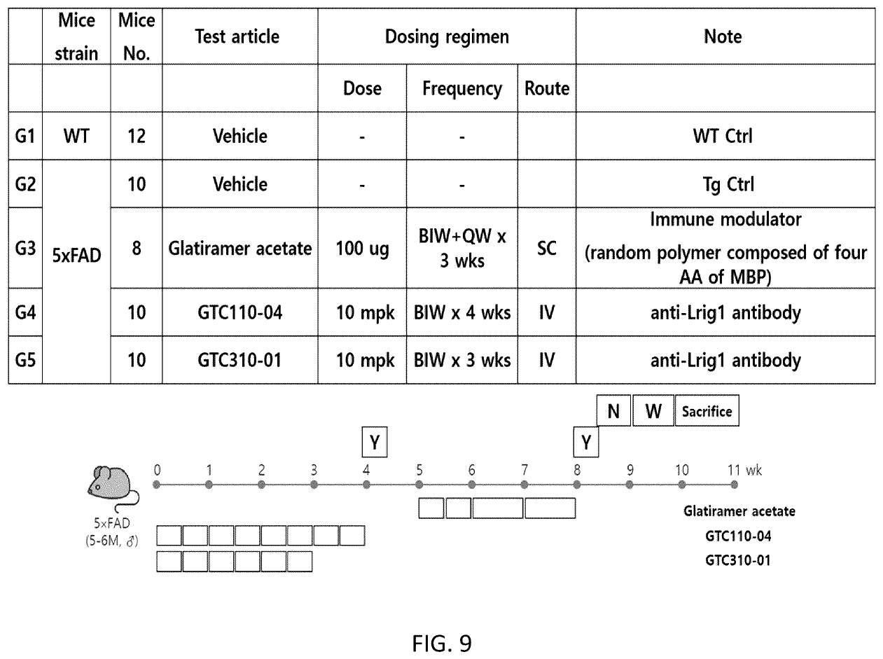

The respective differentiated T cell subsets of Preparation Example 1 were stained with anti-CD4-APC and anti-Lrig-1-PE antibodies, and expression levels of Lrig-1 were measured at the respective cell surfaces using a fluorescence-activated cell sorter (FACS). The results are illustrated in FIG. 8.

As illustrated in FIG. 8, Lrig-1 was expressed in an amount of 0.77 to 15.3 in activated T cells, Th1 cells, Th2 cells, Th17 cells, and naive T cells, whereas Lrig-1 was expressed as high as 83.9 in differentiation-induced T cells (iTreg cells).

From the above results, it can be seen that the Lrig-1 protein according to the present invention is not only specifically expressed in regulatory T (Treg) cells, but also is, in particular, expressed at a higher level on the surface of the Treg cells.

### [Example 6] Evaluation of Therapeutic Ability of Antibody According to Present Invention Against Alzheimer's Disease—5xFAD Mice

In order to evaluate the therapeutic ability of the antibody according to the present invention against Alzheimer's disease, groups were designed as shown in FIG. 9. Specifically, 5- to 6-month-old male 5xFAD mice with induced Alzheimer's disease were intravenously injected with GTC310-01 mouse antibody at an amount of 10 mpk each for 3 weeks. However, glatiramer acetate (GA, Copaxone®) was subcutaneously injected at an amount of 100 μg for 3 weeks, or the H6 antibody (GTC110-04) shown in Table 4 was intravenously injected in an amount of 10 mpk for 4 weeks as a positive control.

TABLE 4

| Clone | Location | Amino Acid Sequence |
|---|---|---|
| H6 | Heavy | EVQLLESGGGLVQPGGSLRLSCAASGFTFSNYAMSWVRQAPGKG |
| antibody | chain | LEWVSVISHGGGSTYYADSVKGRFTISRDNSKNTLYLQMNSLRA |
| (GTC110-04) | | EDTAVYYCARVISNCHLGVCYYSNGMDVWGQGTLVTVSSTTAPS |
| | | VYPLAPVCGDTTGSSVTLGCLVKGYFPEPVTLTWNSGSLSSGVH |
| | | TFPAVLQSDLYTLSSSVTVTSSTWPSQSITCNVAHPASSTKVDK |
| | | KIEPRGPTIKPCPPCKCPAPNLLGGPSVFIFPPKIKDVLMISLS |
| | | PIVTCVVVDVSEDDPDVQISWFVNNVEVHTAQTQTHREDYNSTL |
| | | RVVSALPIQHQDWMSGKEFKCKVNNKDLPAPIERTISKPKGSVR |
| | | APQVYVLPPPEEEMTKKQVTLTCMVTDFMPEDIYVEWTNNGKTE |
| | | LNYKNTEPVLDSDGSYFMYSKLRVEKKNWVERNSYSCSVVHEGL |
| | | HNHHTTKSFSRTPGK |
| | Light | QSVLTQPPSASGTPGQRVTISCSGSSSNIGNNDVYWYQQLPGTA |
| | chain | PKLLIYSDSQRPSGVPDRFSGSKSGTSASLAISGLRSEDEADYY |
| | | CGTWDYSLSGYVFGGGTKLTVLRTVAAPTVSIFPPSSEQLTSGG |
| | | ASVVCFLNNFYPKDINVKWKIDGSERQNGVLNSWTDQDSKDSTY |
| | | SMSSTLTLTKDEYERHNSYTCEATHKTSTSPIVKSFNRNEC |

### 1. Amyloid-β Plaque Reducing Effect

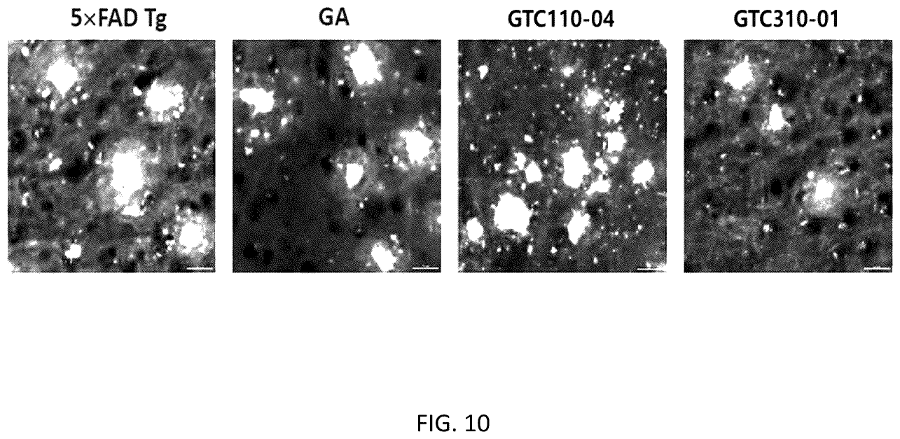

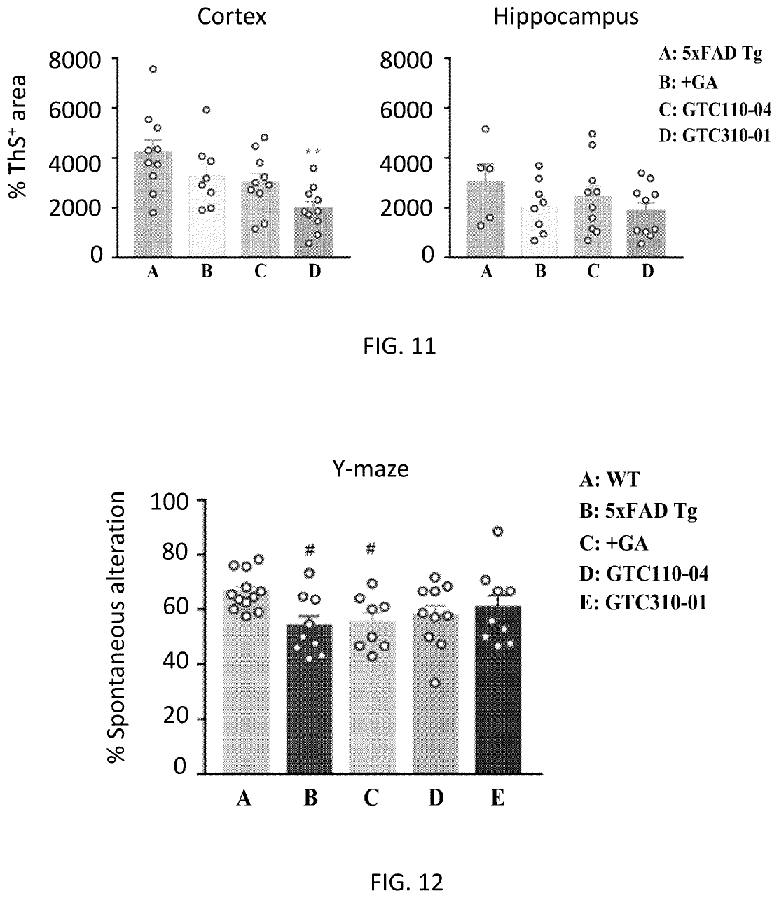

For immunohistological analysis, the above-treated mice were euthanized, and the cortex and hippocampus tissues were obtained and fixed in 4% paraformaldehyde (pH 7.4) for 16 hours. The fixed cortex and hippocampus tissues were immersed in 30% sucrose for cryoprotection and sliced to a thickness of 35 μm. To visualize AR plauqes, the sliced brain sections were stained with thioflavin S (ThS) for 7 minutes. Thioflavin S was purchased from Sigma-Aldrich (catalog number T-1892). Thioflavin S (ThS) at 500 μM was dissolved in 50% ethanol. After successive washes with 100%, 95% and 70% ethanol, the sections were transferred to PBS. Relatively thick free-floating brain slices were incubated with goat anti-GFAP antibody for 7 days at 4° C. for immunofluorescence. Then, the sections were incubated overnight at 4° C. with Alexa Fluor 594-conjugated donkey anti-goat IgG (1:200, Abcam, ab150132). Nuclear counterstaining was performed using 4′6′-damiino-2-phenylindole dihydrochloride hydrate (DAPI, 1 mg/mL, 1:2000, Sigma). Images were taken with a Leica DM2500 fluorescence microscope, and the results are shown in FIG. 10. Additionally, the ThS+ area ratio (%) relative to normal mice was calculated using the ImageJ software program, and the results are shown in FIG. 11.

As shown in FIGS. 10 and 11, although the ThS+ area significantly increased in Alzheimer's-induced mice, when the GTC310-01 antibody according to the present invention was administered, the ThS+ area was significantly reduced, especially in the cortex, thus showing that the effect of reducing amyloid-β plaques was excellent.

### 2. Y-Maze Test

For the Y-maze test, a Y-shaped maze was used which had three identical arms having a length of 40 cm (with a 15-cm high wall) at an angle of 120 degrees from each other. This experiment was a behavioral experiment using the rodent's instinctive exploring habit and was based on the fact that there is a high possibility of exploring a new area. The higher the degree that the test animal remembered the arm it had just explored and tried not to enter the same arm, the higher the animal's memory level. An exploring time of 8 minutes was given to each individual, and the final results were expressed as spontaneous alteration (%) values in FIG. 12. The spontaneous alteration (%) value was calculated by Expression 1. Here, behavioral patterns were analyzed using SMART VIDEO TRACKING Software (Panlab, USA).

$$Spontaneous alteration ( \% ) = \frac{number of triplet}{total arm entry - 2} \qquad [ Equation 1 ]$$

As shown in FIG. 12, in this experiment for measuring the relative frequency at which the test animal perceives the surrounding clues and sequentially enters the maze, it was found that in the case where the mice with induced Alzheimer's disease were administered with the GTC310-01 antibody according to the present invention, the mice had a spontaneous alteration value at a level similar to that of a normal control.

### 3. Novel Object Recognition

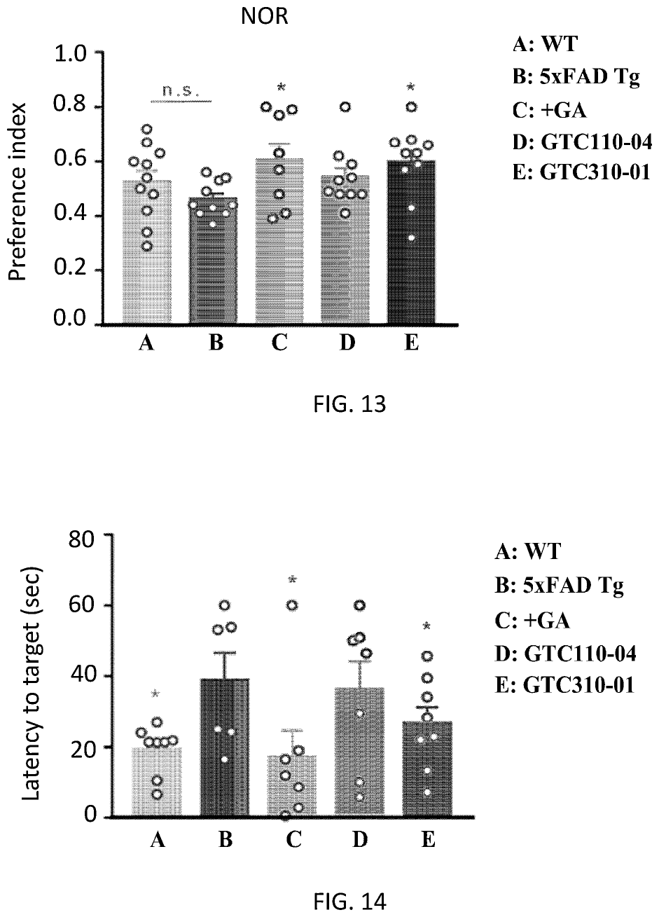

The novel object recognition test was performed to evaluate memory using two different objects in a 40 cm×40 cm acrylic cage. After causing the test animal to acclimatize to the inside of the acrylic cage, two objects were placed at certain locations and the animal was allowed to perceive the objects freely. Then, the time for exploring each object was measured. 24-hour delay was given to each individual, and only one object was changed to another at the same location. Here, in a case where the animal perceives the changed object as a novel object and spends a longer exploring time, it may be determined that the animal has better memory. In a case where the animal does not remember the objects that it explored 24 hours before, the animal fails to distinguish between the novel object and the old object, and thus there is a possibility that the animal explores equally both objects. The animal was allowed to explore freely for a total of 10 minutes, and the result was expressed as a preference index (which equals novel object exploring time/total exploring time) in FIG. 13. Here, the analysis was performed using SMART VIDEO TRACKING Software (Panlab, USA).

As shown in FIG. 13, the preference index for anew object was lower in the mice with induced Alzheimer's disease compared with the normal control. However, when the GTC310-01 antibody according to the present invention was administered, the preference value was higher than that of the normal control.

### 4. Water Maze Test

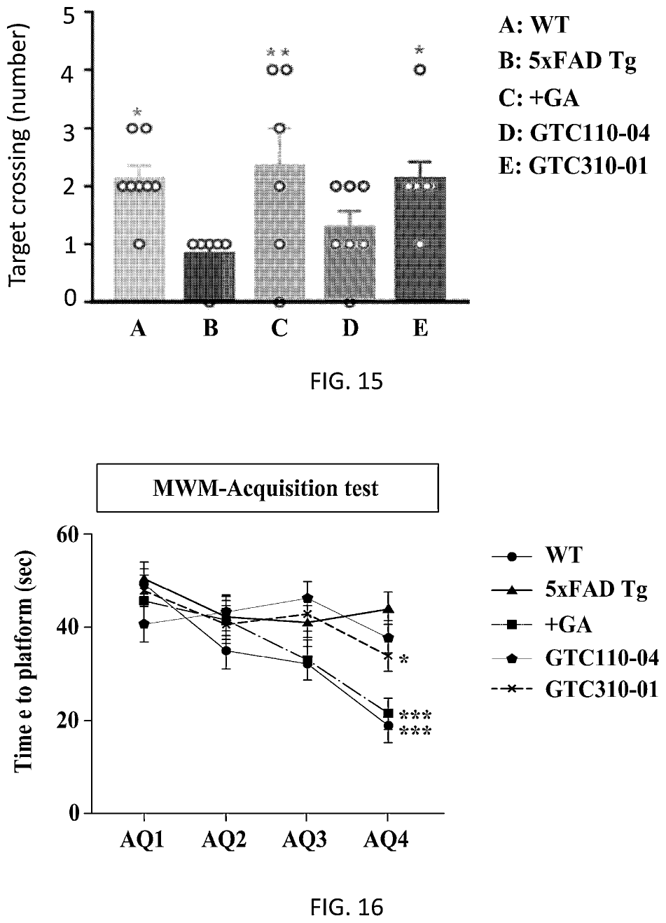

The water maze test was conducted based on the method devised by Morris. A stainless-steel pool (90 cm in diameter, 50 cm in height) was filled with water (22±1° C.) so that the height of the water surface was 30 cm. A hidden platform (5 cm in diameter) was placed 1 cm below the water surface. On day 1 of evaluation, 3 to 4 training sessions were allowed. A total swimming time per individual was set to 60 seconds; and the individual, who found the platform within 60 seconds, was allowed to stay on the platform for 10 seconds to induce memory. The individual, who was unable to find the platform even after 60 seconds, was manually guided to the platform and allowed to stay on the platform for 10 seconds, wherein the time to escape was set to 60 seconds. On day 6 after the training, a probe test was conducted to calculate spatial perception (time taken to reach the location of the platform (latency to target, see)) and the number of times it crossed the target (target crossing numbers, number), the results of which are shown in FIGS. 14 and 15, and an acquisition test was conducted to calculate the time taken to reach the platform (time to platform, see), the result of which is shown in FIG. 16. The analysis was performed using SMART VIDEO TRACKING Software (Panlab, USA). In interpreting the results, the individual included in the exclusion criteria was set as an individual who moves around a specific area without exploration through swimming.

As shown in FIGS. 14 to 16, when the GTC310-01 antibody according to the present invention was administered, the number of times it crossed the target increased significantly, and the time taken to reach the platform significantly decreased.

Through these experiments, it was found that the antibody according to the present invention had a prophylactic, ameliorating, or therapeutic effect on brain and nervous system diseases.

### [Example 7] Evaluation of Therapeutic Effect of Antibodies According to the Present Invention for Alzheimer's—6xTg Mice

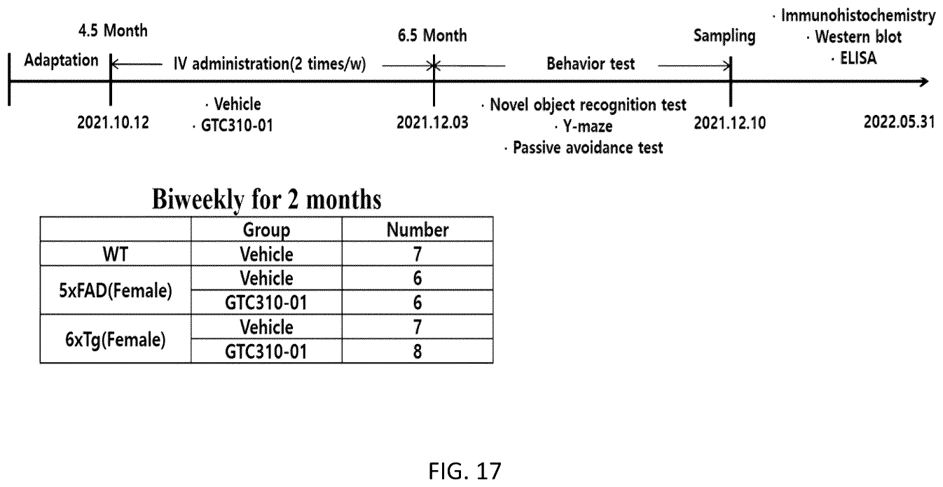

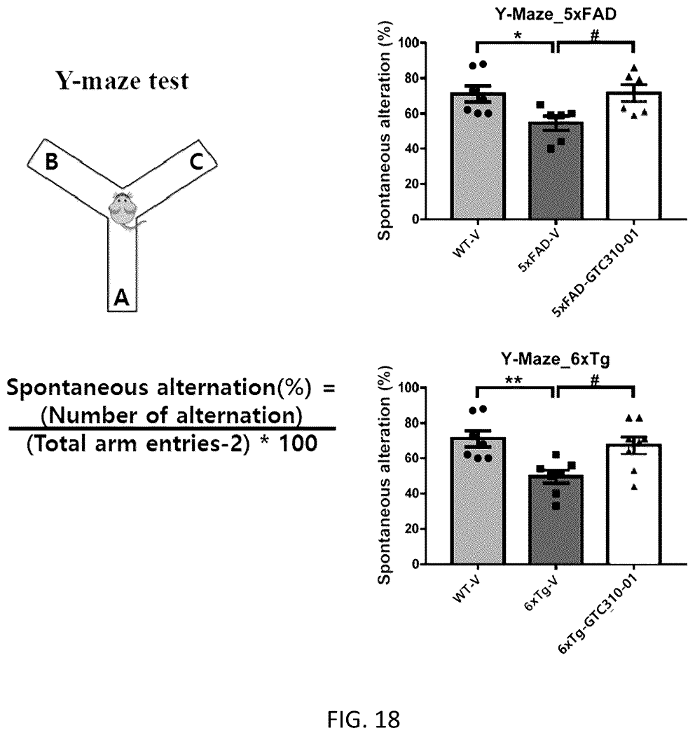

In order to evaluate the therapeutic ability of the antibody according to the present invention against Alzheimer's disease, groups were designed as shown in FIG. 17. Specifically, female 5xFAD mice with induced Alzheimer's disease and female 6xTg mice with induced Alzheimer's disease that underwent an adaptation period of 4.5 months were intravenously injected with GTC310-01 mouse antibody of Preparation Example 1 at an amount of 10 mpk for 2 months. However, glatiramer acetate (GA, Copaxone®) was intravenously injected at an amount of 100 μg for 2 months as a positive control. After 2 months, the Y-maze test, novel object recognition experiment, and passive avoidance test were conducted.

### 1. Characteristics of 6xTg Mice

Although the 5xFAD mice is widely used as an Alzheimer's-induced model, thee 6xTg mice used in the following experiment is a mouse model in which not only amyloid-β (Aβ42) but also tau protein (MAPT) is mutated and accumulated, and thus is a model that is closer to a human disease model than the 5xFAD mouse, thus making it an ideal animal model for neurological diseases including Alzheimer's.

### 2. Y-Maze Test

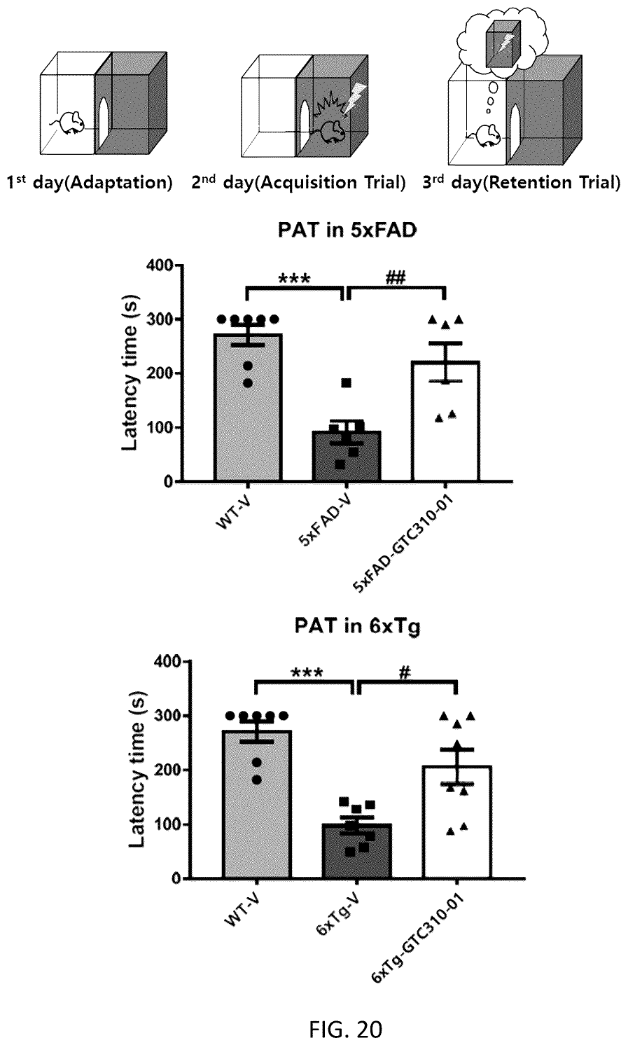

For the Y-maze test, a Y-shaped maze was used which had three identical arms having a length of 40 cm (with a 15-cm high wall) at an angle of 120 degrees from each other. This experiment was a behavioral experiment using the rodent's instinctive exploring habit and was based on the fact that there is a high possibility of exploring a new area. The higher the degree that the test animal remembered the arm it had just explored and tried not to enter the same arm, the higher the animal's memory level. An exploring time of 8 minutes was given to each individual, and the final results were expressed as spontaneous alteration (%) values in FIG. 20. The spontaneous alteration (%) value was calculated by Expression 1. Here, behavioral patterns were analyzed using SMART VIDEO TRACKING Software (Panlab, USA).

$$Spontaneous alteration ( \% ) = \frac{number of triplet}{total arm entry - 2} \qquad [ Equation 1 ]$$

As shown in FIG. 18, in this experiment for measuring the relative frequency at which the test animal perceives the surrounding clues and sequentially enters the maze, it was found that in the case where the mice with induced Alzheimer's disease were administered with the GTC310-01 antibody according to the present invention, the mice had a spontaneous alteration value at a level similar to that of a normal control. Furthermore, mice of the 6xTg model, which is more similar to the human disease model, had a spontaneous alteration value more similar to that of the normal control group than the 5xFAD mouse.

### 3. Novel Object Recognition

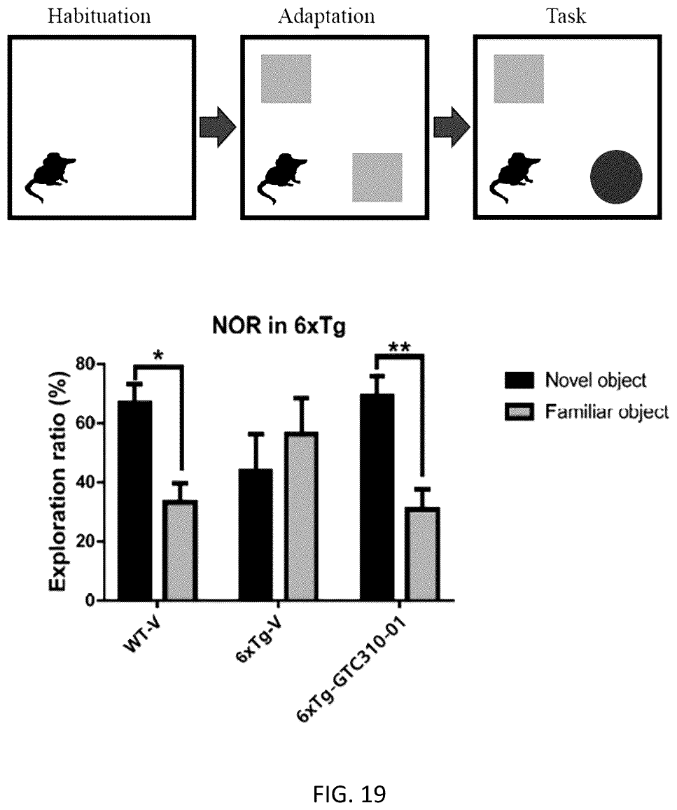

The novel object recognition test was performed to evaluate memory using two different objects in a 40 cm×40 cm acrylic cage. After causing the test animal to acclimatize to the inside of the acrylic cage, two objects were placed at certain locations and the animal was allowed to perceive the objects freely. Then, the time for exploring each object was measured. 24-hour delay was given to each individual, and only one object was changed to another at the same location. Here, in a case where the animal perceives the changed object as a novel object and spends a longer exploring time, it may be determined that the animal has better memory. In a case where the animal does not remember the objects that it explored 24 hours before, the animal fails to distinguish between the novel object and the old object, and thus there is a possibility that the animal explores equally both objects. The animal was allowed to explore freely for a total of 10 minutes, and the result was expressed as a preference index (which equals novel object exploring time/total exploring time) in FIG. 19. Here, the analysis was performed using SMART VIDEO TRACKING Software (Panlab, USA).

As shown in FIG. 19, the preference index for anew object was lower in the mice with induced Alzheimer's disease compared with the normal control. However, when the GTC310-01 antibody according to the present invention was administered, the preference value was higher than that of the normal control in the 6xTg model, which is closer to the human disease model.

### 4. Passive Avoidance Test

The passive avoidance test experimental equipment is divided into two zones, a bright chamber with lighting and a dark chamber, and the floor is made of wire mesh. On the first day, the mice were allowed to freely move and adapted. The following day, each of the 5×FAD mice and 6xTg mice were adapted in a lighted chamber without the lights on for 1 minutes, then the lights were turned on and the mice were adapted for 2 minutes, and as soon as the mice were moved to the dark chamber, an electric shock was applied at 0.5 mA for 1 second to conduct a learning test. A teat trial was conducted for each mouse on the day following the learning test. 5xFAD mice and 6xTg mice were placed in the chamber with lights on, and the time taken for all four paws of the 5xFAD mice and 6xTg mice to enter (latency time) was specified to within 300 seconds. As a result, as shown in FIG. 20, when the GTC310-01 antibody was administered to 5xFAD mice as well as 6xTg mice, the recovery was close to that of the normal control group.

Although the present invention has been described in detail above, the scope of the present invention is not limited thereto. It will be obvious to those skilled in the art that various modifications and changes can be made without departing from the technical spirit of the present invention described in the claims.

### INDUSTRIAL APPLICABILITY

The present invention relates to a novel binding molecule that is excellent for preventing, ameliorating or treating a brain and nervous system disease, and uses thereof to prevent or treat brain and nervous system diseases, particularly neurodegenerative diseases or neuroinflammatory diseases.

Sequence List Free Text

| | |
|---|---|
| <110> | Good T Cells, Inc. |
| <120> | Novel binding molecule and use thereof |
| <130> | POPB214473PCT |
| <160> | 54 |
| <170> | KoPatentIn 3.0 |
| <210> | 1 |
| <211> | 759 |
| <212> | PRT |
| <213> | Homo sapiens |
| <400> | 1 |

```
Gly Pro Arg Ala Pro Cys Ala Ala Ala Cys Thr Cys Ala Gly Asp Ser
1 5 10 15
Leu Asp Cys Gly Gly Arg Gly Leu Ala Ala Leu Pro Gly Asp Leu Pro
                        20 25 30
Ser Trp Thr Arg Ser Leu Asn Leu Ser Tyr Asn Lys Leu Ser Glu Ile
                35 40 45
Asp Pro Ala Gly Phe Glu Asp Leu Pro Asn Leu Gln Glu Val Tyr Leu
        50 55 60
Asn Asn Asn Glu Leu Thr Ala Val Pro Ser Leu Gly Ala Ala Ser Ser
65 70 75 80
His Val Val Ser Leu Phe Leu Gln His Asn Lys Ile Arg Ser Val Glu
                                85 90 95
Gly Ser Gln Leu Lys Ala Tyr Leu Ser Leu Glu Val Leu Asp Leu Ser
                        100 105 110
Leu Asn Asn Ile Thr Glu Val Arg Asn Thr Cys Phe Pro His Gly Pro
                115 120 125
Pro Ile Lys Glu Leu Asn Leu Ala Gly Asn Arg Ile Gly Thr Leu Glu
        130 135 140
Leu Gly Ala Phe Asp Gly Leu Ser Arg Ser Leu Leu Thr Leu Arg Leu
145 150 155 160
Ser Lys Asn Arg Ile Thr Gln Leu Pro Val Arg Ala Phe Lys Leu Pro
                                165 170 175
Arg Leu Thr Gln Leu Asp Leu Asn Arg Asn Arg Ile Arg Leu Ile Glu
                        180 185 190
Gly Leu Thr Phe Gln Gly Leu Asn Ser Leu Glu Val Leu Lys Leu Gln
                195 200 205
Arg Asn Asn Ile Ser Lys Leu Thr Asp Gly Ala Phe Trp Gly Leu Ser
        210 215 220
Lys Met His Val Leu His Leu Glu Tyr Asn Ser Leu Val Glu Val Asn
225 230 235 240
Ser Gly Ser Leu Tyr Gly Leu Thr Ala Leu His Gln Leu His Leu Ser
                                245 250 255
Asn Asn Ser Ile Ala Arg Ile His Arg Lys Gly Trp Ser Phe Cys Gln
                        260 265 270
Lys Leu His Glu Leu Val Leu Ser Phe Asn Asn Leu Thr Arg Leu Asp
                275 280 285
Glu Glu Ser Leu Ala Glu Leu Ser Ser Leu Ser Val Leu Arg Leu Ser
        290 295 300
His Asn Ser Ile Ser His Ile Ala Glu Gly Ala Phe Lys Gly Leu Arg
305 310 315 320
Ser Leu Arg Val Leu Asp Leu Asp His Asn Glu Ile Ser Gly Thr Ile
                                325 330 335
Glu Asp Thr Ser Gly Ala Phe Ser Gly Leu Asp Ser Leu Ser Lys Leu
                        340 345 350
Thr Leu Phe Gly Asn Lys Ile Lys Ser Val Ala Lys Arg Ala Phe Ser
                355 360 365
Gly Leu Glu Gly Leu Glu His Leu Asn Leu Gly Gly Asn Ala Ile Arg
        370 375 380
Ser Val Gln Phe Asp Ala Phe Val Lys Met Lys Asn Leu Lys Glu Leu
385 390 395 400
His Ile Ser Ser Asp Ser Phe Leu Cys Asp Cys Gln Leu Lys Trp Leu
                                405 410 415
Pro Pro Trp Leu Ile Gly Arg Met Leu Gln Ala Phe Val Thr Ala Thr
                        420 425 430
Cys Ala His Pro Glu Ser Leu Lys Gly Gln Ser Ile Phe Ser Val Pro
                435 440 445
Pro Glu Ser Phe Val Cys Asp Asp Phe Leu Lys Pro Gln Ile Ile Thr
        450 455 460
Gln Pro Glu Thr Thr Met Ala Met Val Gly Lys Asp Ile Arg Phe Thr
465 470 475 480
Cys Ser Ala Ala Ser Ser Ser Ser Ser Pro Met Thr Phe Ala Trp Lys
                                485 490 495
Lys Asp Asn Glu Val Leu Thr Asn Ala Asp Met Glu Asn Phe Val His
                        500 505 510
Val His Ala Gln Asp Gly Glu Val Met Glu Tyr Thr Thr Ile Leu His
                515 520 525
Leu Arg Gln Val Thr Phe Gly His Glu Gly Arg Tyr Gln Cys Val Ile
        530 535 540
Thr Asn His Phe Gly Ser Thr Tyr Ser His Lys Ala Arg Leu Thr Val
545 550 555 560
Asn Val Leu Pro Ser Phe Thr Lys Thr Pro His Asp Ile Thr Ile Arg
                                565 570 575
Thr Thr Thr Val Ala Arg Leu Glu Cys Ala Ala Thr Gly His Pro Asn
                        580 585 590
Pro Gln Ile Ala Trp Gln Lys Asp Gly Gly Thr Asp Phe Pro Ala Ala
                595 600 605
Arg Glu Arg Arg Met His Val Met Pro Asp Asp Asp Val Phe Phe Ile
        610 615 620
Thr Asp Val Lys Ile Asp Asp Ala Gly Val Tyr Ser Cys Thr Ala Gln
625 630 635 640
Asn Ser Ala Gly Ser Ile Ser Ala Asn Ala Thr Leu Thr Val Leu Glu
                                645 650 655
Thr Pro Ser Leu Val Val Pro Leu Glu Asp Arg Val Val Ser Val Gly
                        660 665 670
Glu Thr Val Ala Leu Gln Cys Lys Ala Thr Gly Asn Pro Pro Pro Arg
                675 680 685
Ile Thr Trp Phe Lys Gly Asp Arg Pro Leu Ser Leu Thr Glu Arg His
        690 695 700
His Leu Thr Pro Asp Asn Gln Leu Leu Val Val Gln Asn Val Val Ala
705 710 715 720
Glu Asp Ala Gly Arg Tyr Thr Cys Glu Met Ser Asn Thr Leu Gly Thr
                                725 730 735
Glu Arg Ala His Ser Gln Leu Ser Val Leu Pro Ala Ala Gly Cys Arg
                        740 745 750
Lys Asp Gly Thr Thr Val Gly
                755
```

| | |
|---|---|
| <210> | 2 |
| <211> | 2397 |
| <212> | DNA |
| <213> | Homo sapiens |
| <400> | 2 |

| | |
|---|---|
| ggcccgcggg cgccctgcgc ggccgcctgc acttgcgctg gggactcgct ggactgcggt | 60 |
| gggcgcgggc tggctgcgtt gcccggggac ctgccctcct ggacgcggag cctaaacctg | 120 |
| agttacaaca aactctctga gattgaccct gctggttttg aggacttgcc gaacctacag | 180 |
| gaagtgtacc tcaataataa tgagttgaca gcggtaccat ccctgggcgc tgcttcatca | 240 |
| catgtcgtct ctctctttct gcagcacaac aagattcgca gcgtggaggg gagccagctg | 300 |
| aaggcctacc tttccttaga agtgttagat ctgagtttga acaacatcac ggaagtgcgg | 360 |
| aacacctgct ttccacacgg accgcctata aaggagctca acctggcagg caatcggatt | 420 |
| ggcaccctgg agttgggagc atttgatggt ctgtcacggt cgctgctaac tcttcgcctg | 480 |
| agcaaaaaca ggatcaccca gcttcctgta agagcattca agctacccag gctgacacaa | 540 |
| ctggacctca atcggaacag gattcggctg atagagggcc tcaccttcca ggggctcaac | 600 |
| agcttggagg tgctgaagct tcagcgaaac aacatcagca aactgacaga tggggccttc | 660 |
| tggggactgt ccaagatgca tgtgctgcac ctggagtaca acagcctggt agaagtgaac | 720 |
| agcggctcgc tctacggcct cacggccctg catcagctcc acctcagcaa caattccatc | 780 |
| gctcgcattc accgcaaggg ctggagcttc tgccagaagc tgcatgagtt ggtcctgtcc | 840 |
| ttcaacaacc tgacacggct ggacgaggag agcctggccg agctgagcag cctgagtgtc | 900 |
| ctgcgtctca gccacaattc catcagccac attgcggagg gtgccttcaa gggactcagg | 960 |
| agcctgcgag tcttggatct ggaccataac gagatttcgg gcacaataga ggacacgagc | 1020 |
| ggcgccttct cagggctcga cagcctcagc aagctgactc tgtttggaaa caagatcaag | 1080 |
| tctgtggcta agagagcatt ctcggggctg gaaggcctgg agcacctgaa ccttggaggg | 1140 |
| aatgcgatca gatctgtcca gtttgatgcc tttgtgaaga tgaagaatct taaagagctc | 1200 |
| catatcagca gcgacagctt cctgtgtgac tgccagctga agtggctgcc cccgtggcta | 1260 |
| attggcagga tgctgcaggc ctttgtgaca gccacctgtg cccacccaga atcactgaag | 1320 |
| ggtcagagca ttttctctgt gccaccagag agtttcgtgt gcgatgactt cctgaagcca | 1380 |
| cagatcatca cccagccaga aaccaccatg gctatggtgg gcaaggacat ccggtttaca | 1440 |
| tgctcagcag ccagcagcag cagctccccc atgacctttg cctggaagaa agacaatgaa | 1500 |
| gtcctgacca atgcagacat ggagaacttt gtccacgtcc acgcgcagga cggggaagtg | 1560 |
| atggagtaca ccaccatcct gcacctccgt caggtcactt tcgggcacga gggccgctac | 1620 |
| caatgtgtca tcaccaacca ctttggctcc acctattcac ataaggccag gctcaccgtg | 1680 |
| aatgtgttgc catcattcac caaaacgccc cacgacataa ccatccggac caccaccgtg | 1740 |
| gcccgcctcg aatgtgctgc cacaggtcac ccaaaccctc agattgcctg gcagaaggat | 1800 |
| ggaggcacgg atttccccgc tgcccgtgag cgacgcatgc atgtcatgcc ggatgacgac | 1860 |
| gtgtttttca tcactgatgt gaaaatagat gacgcagggg tttacagctg tactgctcag | 1920 |
| aactcagccg gttctatttc agctaatgcc accctgactg tcctagagac cccatccttg | 1980 |
| gtggtcccct tggaagaccg tgtggtatct gtgggagaaa cagtggccct ccaatgcaaa | 2040 |
| gccacgggga accctccgcc ccgcatcacc tggttcaagg gggaccgccc gctgagcctc | 2100 |
| actgagcggc accacctgac ccctgacaac cagctcctgg tggttcagaa cgtggtggca | 2160 |
| gaggatgcgg gccgatatac ctgtgagatg tccaacaccc tgggcacgga gcgagctcac | 2220 |
| agccagctga gcgtcctgcc cgcagcaggc tgcaggaagg atgggaccac ggtaggcatc | 2280 |
| ttcaccattg ctgtcgtgag cagcatcgtc ctgacgtcac tggtctgggt gtgcatcatc | 2340 |
| taccagacca ggaagaagag tgaagagtac agtgtcacca acacagatga aaccgtc | 2397 |

| | |
|---|---|
| <210> | 3 |
| <211> | 761 |
| <212> | PRT |
| <213> | Mus musculus |
| <400> | 3 |

```
Gln Ala Gly Pro Arg Ala Pro Cys Ala Ala Ala Cys Thr Cys Ala Gly
1 5 10 15
Asp Ser Leu Asp Cys Ser Gly Arg Gly Leu Ala Thr Leu Pro Arg Asp
                        20 25 30
Leu Pro Ser Trp Thr Arg Ser Leu Asn Leu Ser Tyr Asn Arg Leu Ser
                35 40 45
Glu Ile Asp Ser Ala Ala Phe Glu Asp Leu Thr Asn Leu Gln Glu Val
        50 55 60
Tyr Leu Asn Ser Asn Glu Leu Thr Ala Ile Pro Ser Leu Gly Ala Ala
65 70 75 80
Ser Ile Gly Val Val Ser Leu Phe Leu Gln His Asn Lys Ile Leu Ser
                                85 90 95
Val Asp Gly Ser Gln Leu Lys Ser Tyr Leu Ser Leu Glu Val Leu Asp
                        100 105 110
Leu Ser Ser Asn Asn Ile Thr Glu Ile Arg Ser Ser Cys Phe Pro Asn
                115 120 125
Gly Leu Arg Ile Arg Glu Leu Asn Leu Ala Ser Asn Arg Ile Ser Ile
        130 135 140
Leu Glu Ser Gly Ala Phe Asp Gly Leu Ser Arg Ser Leu Leu Thr Leu
145 150 155 160
Arg Leu Ser Lys Asn Arg Ile Thr Gln Leu Pro Val Lys Ala Phe Lys
                                165 170 175
Leu Pro Arg Leu Thr Gln Leu Asp Leu Asn Arg Asn Arg Ile Arg Leu
                        180 185 190
Ile Glu Gly Leu Thr Phe Gln Gly Leu Asp Ser Leu Glu Val Leu Arg
                195 200 205
Leu Gln Arg Asn Asn Ile Ser Arg Leu Thr Asp Gly Ala Phe Trp Gly
        210 215 220
Leu Ser Lys Met His Val Leu His Leu Glu Tyr Asn Ser Leu Val Glu
225 230 235 240
Val Asn Ser Gly Ser Leu Tyr Gly Leu Thr Ala Leu His Gln Leu His
                                245 250 255
Leu Ser Asn Asn Ser Ile Ser Arg Ile Gln Arg Asp Gly Trp Ser Phe
                        260 265 270
Cys Gln Lys Leu His Glu Leu Ile Leu Ser Phe Asn Asn Leu Thr Arg
                275 280 285
Leu Asp Glu Glu Ser Leu Ala Glu Leu Ser Ser Leu Ser Ile Leu Arg
        290 295 300
Leu Ser His Asn Ala Ile Ser His Ile Ala Glu Gly Ala Phe Lys Gly
305 310 315 320
Leu Lys Ser Leu Arg Val Leu Asp Leu Asp His Asn Glu Ile Ser Gly
                                325 330 335
Thr Ile Glu Asp Thr Ser Gly Ala Phe Thr Gly Leu Asp Asn Leu Ser
                        340 345 350
Lys Leu Thr Leu Phe Gly Asn Lys Ile Lys Ser Val Ala Lys Arg Ala
                355 360 365
Phe Ser Gly Leu Glu Ser Leu Glu His Leu Asn Leu Gly Glu Asn Ala
        370 375 380
Ile Arg Ser Val Gln Phe Asp Ala Phe Ala Lys Met Lys Asn Leu Lys
385 390 395 400
Glu Leu Tyr Ile Ser Ser Glu Ser Phe Leu Cys Asp Cys Gln Leu Lys
                                405 410 415
Trp Leu Pro Pro Trp Leu Met Gly Arg Met Leu Gln Ala Phe Val Thr
                        420 425 430
Ala Thr Cys Ala His Pro Glu Ser Leu Lys Gly Gln Ser Ile Phe Ser
                435 440 445
Val Leu Pro Asp Ser Phe Val Cys Asp Asp Phe Pro Lys Pro Gln Ile
        450 455 460
Ile Thr Gln Pro Glu Thr Thr Met Ala Val Val Gly Lys Asp Ile Arg
465 470 475 480
Phe Thr Cys Ser Ala Ala Ser Ser Ser Ser Ser Pro Met Thr Phe Ala
                                485 490 495
Trp Lys Lys Asp Asn Glu Val Leu Ala Asn Ala Asp Met Glu Asn Phe
                        500 505 510
Ala His Val Arg Ala Gln Asp Gly Glu Val Met Glu Tyr Thr Thr Ile
                515 520 525
Leu His Leu Arg His Val Thr Phe Gly His Glu Gly Arg Tyr Gln Cys
        530 535 540
Ile Ile Thr Asn His Phe Gly Ser Thr Tyr Ser His Lys Ala Arg Leu
545 550 555 560
Thr Val Asn Val Leu Pro Ser Phe Thr Lys Ile Pro His Asp Ile Ala
                                565 570 575
Ile Arg Thr Gly Thr Thr Ala Arg Leu Glu Cys Ala Ala Thr Gly His
                        580 585 590
Pro Asn Pro Gln Ile Ala Trp Gln Lys Asp Gly Gly Thr Asp Phe Pro
                595 600 605
Ala Ala Arg Glu Arg Arg Met His Val Met Pro Asp Asp Asp Val Phe
        610 615 620
Phe Ile Thr Asp Val Lys Ile Asp Asp Met Gly Val Tyr Ser Cys Thr
625 630 635 640
Ala Gln Asn Ser Ala Gly Ser Val Ser Ala Asn Ala Thr Leu Thr Val
                                645 650 655
Leu Glu Thr Pro Ser Leu Ala Val Pro Leu Glu Asp Arg Val Val Thr
                        660 665 670
Val Gly Glu Thr Val Ala Phe Gln Cys Lys Ala Thr Gly Ser Pro Thr
                675 680 685
Pro Arg Ile Thr Trp Leu Lys Gly Gly Arg Pro Leu Ser Leu Thr Glu
        690 695 700
Arg His His Phe Thr Pro Gly Asn Gln Leu Leu Val Val Gln Asn Val
705 710 715 720
Met Ile Asp Asp Ala Gly Arg Tyr Thr Cys Glu Met Ser Asn Pro Leu
                                725 730 735
Gly Thr Glu Arg Ala His Ser Gln Leu Ser Ile Leu Pro Thr Pro Gly
                        740 745 750
Cys Arg Lys Asp Gly Thr Thr Val Gly
                755 760
```

| | |
|---|---|
| <210> | 4 |
| <211> | 2283 |
| <212> | DNA |
| <213> | Mus musculus |
| <400> | 4 |

| | |
|---|---|
| caggctggcc cgcgggcccc ctgcgcggcc gcctgcactt gcgccgggga ctcgctggac | 60 |
| tgcagtgggc gcgggctggc gacgctgccc cgggacctgc cctcctggac gcgcagccta | 120 |
| aacctgagtt ataacagact ctccgagatc gactctgctg cttttgagga cttgacgaat | 180 |
| ctgcaggaag tgtacctcaa cagcaatgag ctgacagcca taccatcact gggcgctgct | 240 |
| tccataggag ttgtctctct ctttttgcag cacaacaaga tccttagtgt ggatgggagc | 300 |
| cagctgaagt cgtacctgtc cttggaagtg ctggatctga gttccaacaa catcacggaa | 360 |
| attcggagct cctgtttccc gaacggcctg cgtataaggg aactcaactt ggcgagcaac | 420 |
| cgcatcagca tcctggagtc tggagcattt gatggtctgt cgcggtcact gctgactctc | 480 |
| cgtctgagca aaaacaggat cacccagctt cctgtgaaag cgttcaagct acccaggctg | 540 |
| acacaactag acctgaatcg gaatcggatt cggctgattg aaggcctcac gttccagggg | 600 |
| ctcgacagct tagaggtgct gaggcttcag aggaacaaca tcagcaggct gacggacggg | 660 |
| gccttctggg ggctgtctaa gatgcacgtg ctgcacctgg agtacaacag tctggtggaa | 720 |
| gtgaacagtg gctccctcta tggcctcaca gccctgcacc agctgcacct cagcaacaac | 780 |
| tccatctctc gaattcagcg tgatggctgg agcttctgcc aaaagctgca tgagttgatt | 840 |
| ctgtccttca acaacctcac gcggctggat gaggagagtc tagcggagtt gagcagcctc | 900 |
| agtatcctgc gcctcagtca caacgccatc agtcacattg ctgaaggcgc cttcaaggga | 960 |
| ctcaagagtc tgcgggtctt ggacctggac cataacgaga tctcgggtac aatcgaggat | 1020 |
| accagtggtg cctttacggg gcttgacaac ctcagcaagc tgactctgtt tggaaacaag | 1080 |
| atcaaatctg tggctaagag agccttctcg ggcctggaaa gcctggaaca cctgaacctt | 1140 |
| ggagagaatg caatcaggtc tgtccagttt gatgcctttg caaagatgaa gaaccttaaa | 1200 |
| gagctctaca tcagcagtga gagcttcctg tgtgactgcc agctcaagtg gctgccccca | 1260 |
| tggctaatgg gtaggatgct gcaggccttt gtgacagcca cctgtgccca tccagagtcg | 1320 |
| ctgaagggcc agagcatttt ctcagtgctg ccagacagct ttgtgtgtga tgactttcca | 1380 |
| aagccacaga tcatcaccca gcctgagacg accatggctg tggtgggcaa ggacatccgt | 1440 |
| ttcacatgct ccgcagccag cagcagcagc tcaccaatga ccttcgcctg gaagaaggac | 1500 |
| aatgaggtcc tggccaatgc agacatggag aactttgccc acgtccgtgc acaggacggc | 1560 |
| gaagtgatgg agtataccac tatcctgcac ctccgtcacg tcacctttgg gcacgagggc | 1620 |
| cgctaccagt gtatcatcac aaaccacttt ggctccacat actcccacaa agccaggctc | 1680 |
| actgtgaatg tgttgccatc attcactaaa ataccccatg acattgccat ccggactggc | 1740 |
| accacagccc gcctcgagtg tgctgccacg ggccacccta accctcagat tgcctggcag | 1800 |
| aaggatggag gcaccgattt cccggcagct cgtgagcgac gcatgcatgt tatgccagac | 1860 |
| gatgatgtgt tcttcatcac tgatgtgaaa atagacgaca tgggggtcta cagctgcact | 1920 |
| gcccagaact cggcaggctc ggtttcagcc aacgctaccc tcacagtctt agaaactcca | 1980 |
| tccttggcag tgcctctgga agaccgtgtg gtaactgtgg gagaaacagt ggccttccag | 2040 |
| tgcaaagcaa ccgggagccc cacaccacgc atcacctggc ttaagggagg tcgcccattg | 2100 |
| agcctcacag agcgccacca tttcactcca ggcaaccagc tgctggttgt tcagaatgtg | 2160 |
| atgatagacg atgcagggcg gtatacctgt gagatgtcta atcccctggg cactgagcga | 2220 |
| gcacatagcc agctgagcat tttacctacc cctggctgcc ggaaggatgg gaccaccgta | 2280 |
| ggc | 2283 |

| | |
|---|---|
| <210> | 5 |
| <211> | 5 |
| <212> | PRT |
| <213> | Artificial Sequence |
| <220> | |
| <223> | Heavy chain CDR 1 |
| <400> | 5 |

Asn Tyr Ala Met Ser
1 5

| | |
|---|---|
| <210> | 6 |
| <211> | 17 |
| <212> | PRT |
| <213> | Artificial Sequence |
| <220> | |
| <223> | Heavy chain CDR 2 |
| <400> | 6 |

Val Ile Ser His Gly Gly Gly Ser Thr Tyr Tyr Ala Asp Ser Val Lys
1 5 10 15
Gly

| | |
|---|---|
| <210> | 7 |
| <211> | 18 |
| <212> | PRT |
| <213> | Artificial Sequence |
| <220> | |
| <223> | Heavy chain CDR 3 |
| <400> | 7 |

Val Ile Ser Asn Cys His Leu Gly Val Cys Tyr Tyr Ser Asn Gly Met
1 5 10 15
Asp Val

| | |
|---|---|
| <210> | 8 |
| <211> | 13 |
| <212> | PRT |
| <213> | Artificial Sequence |
| <220> | |
| <223> | Light chain CDR 1 |
| <400> | 8 |

Ser Gly Ser Ser Ser Asn Ile Gly Asp Asn Tyr Val Ser
1 5 10

| | |
|---|---|
| <210> | 9 |
| <211> | 7 |
| <212> | PRT |
| <213> | Artificial Sequence |
| <220> | |
| *884<223> | Light chain CDR 2 |
| <400> | 9 |

Asp Asn Asn Lys Arg Pro Ser
1 5

| | |
|---|---|
| <210> | 10 |
| <211> | 10 |
| <212> | PRT |
| <213> | Artificial Sequence |
| <220> | |
| <223> | Light chain CDR 3 |
| <400> | 10 |

Trp Asp Gly Ser Leu Ser Ala Gly Arg Val
1 5 10

| | |
|---|---|
| <210> | 11 |
| <211> | 127 |
| <212> | PRT |
| <213> | Artificial Sequence |
| <220> | |
| <223> | Heavy chain variable region |
| <400> | 11 |

```
Glu Val Gln Leu Leu Glu Ser Gly Gly Gly Leu Val Gln Pro Gly Gly
1 5 10 15
*918
Ser Leu Arg Leu Ser Cys Ala Ala Ser Gly Phe Thr Phe Ser Asn Tyr
                        20 25 30
Ala Met Ser Trp Val Arg Gln Ala Pro Gly Lys Gly Leu Glu Trp Val
                35 40 45
Ser Val Ile Ser His Gly Gly Gly Ser Thr Tyr Tyr Ala Asp Ser Val
        50 55 60
Lys Gly Arg Phe Thr Ile Ser Arg Asp Asn Ser Lys Asn Thr Leu Tyr
65 70 75 80
*930
Leu Gln Met Asn Ser Leu Arg Ala Glu Asp Thr Ala Val Tyr Tyr Cys
                                85 90 95
Ala Arg Val Ile Ser Asn Cys His Leu Gly Val Cys Tyr Tyr Ser Asn
                        100 105 110
Gly Met Asp Val Trp Gly Gln Gly Thr Leu Val Thr Val Ser Ser
                115 120 125
```

| | |
|---|---|
| <210> | 12 |
| <211> | 111 |
| <212> | PRT |
| <213> | Artificial Sequence |
| <220> | |
| <223> | Light chain variable region |
| <400> | 12 |

```
Gln Leu Val Leu Thr Gln Pro Pro Ser Val Ser Ala Ala Pro Gly Gln
1 5 10 15
*955
Arg Val Ser Ile Ser Cys Ser Gly Ser Ser Ser Asn Ile Gly Asp Asn
                        20 25 30
Tyr Val Ser Trp Tyr Gln Gln Val Pro Gly Ser Ala Pro Lys Leu Leu
                35 40 45
Ile Tyr Asp Asn Asn Lys Arg Pro Ser Gly Ile Pro Asp Arg Phe Ser
        50 55 60
Ala Ser Lys Ser Gly Thr Ser Ala Thr Leu Gly Ile Thr Gly Leu Gln
65 70 75 80
*967
Thr Gly Asp Glu Ala Asp Tyr Tyr Cys Ala Thr Trp Asp Gly Ser Leu
                                85 90 95
Ser Ala Gly Arg Val Phe Gly Gly Gly Thr Lys Leu Thr Val Leu
                        100 105 110
```

| | |
|---|---|
| <210> | 13 |
| <211> | 15 |
| <212> | DNA |
| <213> | Artificial Sequence |
| <220> | |
| <223> | Heavy chain CDR 1 |
| <400> | 13 |

| | |
|---|---|
| aactacgcca tgagc | 15 |

| | |
|---|---|
| <210> | 14 |
| <211> | 51 |
| <212> | DNA |
| <213> | Artificial Sequence |
| <220> | |
| <223> | Heavy chain CDR 2 |
| <400> | 14 |

| | |
|---|---|
| gtgatctctc acggcggagg cagcacctac tacgccgatt ctgtgaaggg c | 51 |

| | |
|---|---|
| <210> | 15 |
| <211> | 54 |
| <212> | DNA |
| <213> | Artificial Sequence |
| <220> | |
| <223> | Heavy chain CDR 3 |
| *1009 | |
| <400> | 15 |

| | |
|---|---|
| gtgatcagca actgccacct gggcgtgtgc tactacagca acggcatgga tgtg | 54 |

| | |
|---|---|
| <210> | 16 |
| <211> | 39 |
| <212> | DNA |
| <213> | Artificial Sequence |
| <220> | |
| <223> | Light chain CDR 1 |
| *1022 | |
| <400> | 16 |

| | |
|---|---|
| agcggcagca gcagcaacat cggcgacaac tacgtgtcc | 39 |

| | |
|---|---|
| <210> | 17 |
| <211> | 21 |
| <212> | DNA |
| <213> | Artificial Sequence |
| <220> | |
| <223> | Light chain CDR 2 |
| *1035 | |
| <400> | 17 |

| | |
|---|---|
| gacaacaaca agcggcccag c | 21 |

| | |
|---|---|
| <210> | 18 |
| <211> | 30 |
| <212> | DNA |
| <213> | Artificial Sequence |
| <220> | |
| <223> | Light chain CDR 3 |
| *1048 | |
| <400> | 18 |

| | |
|---|---|
| tgggatggct ccctgagcgc cggaagagtt | 30 |

| | |
|---|---|
| <210> | 19 |
| <211> | 381 |
| <212> | DNA |
| <213> | Artificial Sequence |
| <220> | |
| <223> | Heavy chain variable region |
| <400> | 19 |

| | |
|---|---|
| gaagttcagc tgcttgagtc tggcggagga ctggttcaac ctggcggaag cctgagactg | 60 |
| tcttgtgccg ccagcggctt caccttcagc aactacgcca tgagctgggt ccgacaggcc | 120 |
| cctggaaaag gccttgaatg ggtgtccgtg atctctcacg gcggaggcag cacctactac | 180 |
| gccgattctg tgaagggcag attcaccatc agccgggaca acagcaagaa caccctgtac | 240 |
| ctgcagatga acagcctgag agccgaggac accgccgtgt actattgcgc cagagtgatc | 300 |
| agcaactgcc acctgggcgt gtgctactac agcaacggca tggatgtgtg gggccagggc | 360 |
| acactggtta ccgttagttc t | 381 |

| | |
|---|---|
| <210> | 20 |
| <211> | 333 |
| <212> | DNA |
| <213> | Artificial Sequence |
| <220> | |
| <223> | Light chain variable region |
| <400> | 20 |

| | |
|---|---|
| caactggttc tgacacagcc tccaagcgtg tcagctgccc ctggacagag agtgtccatc | 60 |
| agctgtagcg gcagcagcag caacatcggc gacaactacg tgtcctggta tcagcaggtc | 120 |
| ccaggctctg cccctaagct gctgatctac gacaacaaca agcggcccag cggcatcccc | 180 |
| gatagatttt ctgccagcaa gagcggcacc agcgccacac tgggaattac aggactgcag | 240 |
| acaggcgacg aggccgacta ctattgtgcc acatgggatg gctccctgag cgccggaaga | 300 |
| gtttttggcg gaggcaccaa gctgaccgtg ctt | 333 |

| | |
|---|---|
| <210> | 21 |
| <211> | 328 |
| <212> | PRT |
| <213> | Mus musculus |
| <400> | 21 |

```
Thr Thr Ala Pro Ser Val Tyr Pro Leu Ala Pro Val Cys Gly Asp Thr
1 5 10 15
Thr Gly Ser Ser Val Thr Leu Gly Cys Leu Val Lys Gly Tyr Phe Pro
                        20 25 30
Glu Pro Val Thr Leu Thr Trp Asn Ser Gly Ser Leu Ser Ser Gly Val
                35 40 45
His Thr Phe Pro Ala Val Leu Gln Ser Asp Leu Tyr Thr Leu Ser Ser
        50 55 60
Ser Val Thr Val Thr Ser Ser Thr Trp Pro Ser Gln Ser Ile Thr Cys
65 70 75 80
Asn Val Ala His Pro Ala Ser Ser Thr Lys Val Asp Lys Lys Ile Glu
                                85 90 95
Pro Arg Gly Pro Thr Ile Lys Pro Cys Pro Pro Cys Lys Cys Pro Ala
                        100 105 110
Pro Asn Leu Leu Gly Gly Pro Ser Val Phe Ile Phe Pro Pro Lys Ile
                115 120 125
Lys Asp Val Leu Met Ile Ser Leu Ser Pro Ile Val Thr Cys Val Val
        130 135 140
Val Asp Val Ser Glu Asp Asp Pro Asp Val Gln Ile Ser Trp Phe Val
145 150 155 160
Asn Asn Val Glu Val His Thr Ala Gln Thr Gln Thr His Arg Glu Asp
                                165 170 175
Tyr Asn Ser Thr Leu Arg Val Val Ser Ala Leu Pro Ile Gln His Gln
                        180 185 190
Asp Trp Met Ser Gly Lys Glu Phe Lys Cys Lys Val Asn Asn Lys Asp
                195 200 205
Leu Pro Ala Pro Ile Glu Arg Thr Ile Ser Lys Pro Lys Gly Ser Val
        210 215 220
Arg Ala Pro Gln Val Tyr Val Leu Pro Pro Pro Glu Glu Glu Met Thr
225 230 235 240
Lys Lys Gln Val Thr Leu Thr Cys Met Val Thr Asp Phe Met Pro Glu
                                245 250 255
Asp Ile Tyr Val Glu Trp Thr Asn Asn Gly Lys Thr Glu Leu Asn Tyr
                        260 265 270
Lys Asn Thr Glu Pro Val Leu Asp Ser Asp Gly Ser Tyr Phe Met Tyr
                275 280 285
Ser Lys Leu Arg Val Glu Lys Lys Asn Trp Val Glu Arg Asn Ser Tyr
        290 295 300
Ser Cys Ser Val Val His Glu Gly Leu His Asn His His Thr Thr Lys
305 310 315 320
Ser Phe Ser Arg Thr Pro Gly Lys
                                325
```

| | |
|---|---|
| <210> | 22 |
| <211> | 107 |
| <212> | PRT |
| <213> | Mus musculus |
| <400> | 22 |

```
Arg Thr Val Ala Ala Pro Thr Val Ser Ile Phe Pro Pro Ser Ser Glu
1 5 10 15
Gln Leu Thr Ser Gly Gly Ala Ser Val Val Cys Phe Leu Asn Asn Phe
                        20 25 30
Tyr Pro Lys Asp Ile Asn Val Lys Trp Lys Ile Asp Gly Ser Glu Arg
                35 40 45
Gln Asn Gly Val Leu Asn Ser Trp Thr Asp Gln Asp Ser Lys Asp Ser
        50 55 60
Thr Tyr Ser Met Ser Ser Thr Leu Thr Leu Thr Lys Asp Glu Tyr Glu
65 70 75 80
Arg His Asn Ser Tyr Thr Cys Glu Ala Thr His Lys Thr Ser Thr Ser
                                85 90 95
*1195
Pro Ile Val Lys Ser Phe Asn Arg Asn Glu Cys
                        100 105
```

| | |
|---|---|
| <210> | 23 |
| <211> | 330 |
| <212> | PRT |
| <213> | Homo sapiens |
| <400> | 23 |

```
Ala Ser Thr Lys Gly Pro Ser Val Phe Pro Leu Ala Pro Ser Ser Lys
1 5 10 15
Ser Thr Ser Gly Gly Thr Ala Ala Leu Gly Cys Leu Val Lys Asp Tyr
                        20 25 30
Phe Pro Glu Pro Val Thr Val Ser Trp Asn Ser Gly Ala Leu Thr Ser
                35 40 45
Gly Val His Thr Phe Pro Ala Val Leu Gln Ser Ser Gly Leu Tyr Ser
        50 55 60
Leu Ser Ser Val Val Thr Val Pro Ser Ser Ser Leu Gly Thr Gln Thr
65 70 75 80
Tyr Ile Cys Asn Val Asn His Lys Pro Ser Asn Thr Lys Val Asp Lys
                                85 90 95
Lys Val Glu Pro Lys Ser Cys Asp Lys Thr His Thr Cys Pro Pro Cys
                        100 105 110
Pro Ala Pro Glu Leu Leu Gly Gly Pro Ser Val Phe Leu Phe Pro Pro
                115 120 125
Lys Pro Lys Asp Thr Leu Met Ile Ser Arg Thr Pro Glu Val Thr Cys
        130 135 140
Val Val Val Asp Val Ser His Glu Asp Pro Glu Val Lys Phe Asn Trp
145 150 155 160
Tyr Val Asp Gly Val Glu Val His Asn Ala Lys Thr Lys Pro Arg Glu
                                165 170 175
Glu Gln Tyr Asn Ser Thr Tyr Arg Val Val Ser Val Leu Thr Val Leu
                        180 185 190
His Gln Asp Trp Leu Asn Gly Lys Glu Tyr Lys Cys Lys Val Ser Asn
                195 200 205
Lys Ala Leu Pro Ala Pro Ile Glu Lys Thr Ile Ser Lys Ala Lys Gly
        210 215 220
Gln Pro Arg Glu Pro Gln Val Tyr Thr Leu Pro Pro Ser Arg Asp Glu
225 230 235 240
Leu Thr Lys Asn Gln Val Ser Leu Thr Cys Leu Val Lys Gly Phe Tyr
                                245 250 255
Pro Ser Asp Ile Ala Val Glu Trp Glu Ser Asn Gly Gln Pro Glu Asn
                        260 265 270
Asn Tyr Lys Thr Thr Pro Pro Val Leu Asp Ser Asp Gly Ser Phe Phe
                275 280 285
Leu Tyr Ser Lys Leu Thr Val Asp Lys Ser Arg Trp Gln Gln Gly Asn
        290 295 300
Val Phe Ser Cys Ser Val Met His Glu Ala Leu His Asn His Tyr Thr
305 310 315 320
Gln Lys Ser Leu Ser Leu Ser Pro Gly Lys
                                325 330
```

| | |
|---|---|
| <210> | 24 |
| <211> | 330 |
| <212> | PRT |
| <213> | Homo sapiens |
| <400> | 24 |

```
Ala Ser Thr Lys Gly Pro Ser Val Phe Pro Leu Ala Pro Ser Ser Lys
1 5 10 15
Ser Thr Ser Gly Gly Thr Ala Ala Leu Gly Cys Leu Val Lys Asp Tyr
                        20 25 30
Phe Pro Glu Pro Val Thr Val Ser Trp Asn Ser Gly Ala Leu Thr Ser
                35 40 45
Gly Val His Thr Phe Pro Ala Val Leu Gln Ser Ser Gly Leu Tyr Ser
        50 55 60
Leu Ser Ser Val Val Thr Val Pro Ser Ser Ser Leu Gly Thr Gln Thr
65 70 75 80
Tyr Ile Cys Asn Val Asn His Lys Pro Ser Asn Thr Lys Val Asp Lys
                                85 90 95
*1293
Lys Val Glu Pro Lys Ser Cys Asp Lys Thr His Thr Cys Pro Pro Cys
                        100 105 110
Pro Ala Pro Glu Leu Leu Gly Gly Pro Ser Val Phe Leu Phe Pro Pro
                115 120 125
Lys Pro Lys Asp Thr Leu Met Ile Ser Arg Thr Pro Glu Val Thr Cys
        130 135 140
Val Val Val Asp Val Ser His Glu Asp Pro Glu Val Lys Phe Asn Trp
145 150 155 160
Tyr Val Asp Gly Val Glu Val His Asn Ala Lys Thr Lys Pro Arg Glu
                                165 170 175
Glu Gln Tyr Asn Ser Thr Tyr Arg Val Val Ser Val Leu Thr Val Leu
                        180 185 190
His Gln Asp Trp Leu Asn Gly Lys Glu Tyr Lys Cys Lys Val Ser Asn
                195 200 205
Lys Ala Leu Pro Ala Pro Ile Glu Lys Thr Ile Ser Lys Ala Lys Gly
        210 215 220
Gln Pro Arg Glu Pro Gln Val Tyr Thr Leu Pro Pro Ser Arg Glu Glu
225 230 235 240
Met Thr Lys Asn Gln Val Ser Leu Thr Cys Leu Val Lys Gly Phe Tyr
                                245 250 255
Pro Ser Asp Ile Ala Val Glu Trp Glu Ser Asn Gly Gln Pro Glu Asn
                        260 265 270
Asn Tyr Lys Thr Thr Pro Pro Val Leu Asp Ser Asp Gly Ser Phe Phe
                275 280 285
Leu Tyr Ser Lys Leu Thr Val Asp Lys Ser Arg Trp Gln Gln Gly Asn
        290 295 300
Val Phe Ser Cys Ser Val Met His Glu Ala Leu His Asn His Tyr Thr
305 310 315 320
Gln Lys Ser Leu Ser Leu Ser Pro Gly Lys
                                325 330
```

| | |
|---|---|
| <210> | 25 |
| <211> | 107 |
| <212> | PRT |
| <213> | Homo sapiens |
| <400> | 25 |

```
Arg Thr Val Ala Ala Pro Ser Val Phe Ile Phe Pro Pro Ser Asp Glu
1 5 10 15
Gln Leu Lys Ser Gly Thr Ala Ser Val Val Cys Leu Leu Asn Asn Phe
                        20 25 30
Tyr Pro Arg Glu Ala Lys Val Gln Trp Lys Val Asp Asn Ala Leu Gln
                35 40 45
Ser Gly Asn Ser Gln Glu Ser Val Thr Glu Gln Asp Ser Lys Asp Ser
        50 55 60
Thr Tyr Ser Leu Ser Ser Thr Leu Thr Leu Ser Lys Ala Asp Tyr Glu
65 70 75 80
Lys His Lys Val Tyr Ala Cys Glu Val Thr His Gln Gly Leu Ser Ser
                                85 90 95
Pro Val Thr Lys Ser Phe Asn Arg Gly Glu Cys
                        100 105
```

| | |
|---|---|
| <210> | 26 |
| <211> | 107 |
| <212> | PRT |
| <213> | Homo sapiens |
| <400> | 26 |

```
Arg Ser Val Ala Ala Pro Ser Val Phe Ile Phe Pro Pro Ser Asp Glu
1 5 10 15
Gln Leu Lys Ser Gly Thr Ala Ser Val Val Cys Leu Leu Asn Asn Phe
                        20 25 30
Tyr Pro Arg Glu Ala Lys Val Gln Trp Lys Val Asp Asn Ala Leu Gln
                35 40 45
Ser Gly Asn Ser Gln Glu Ser Val Thr Glu Gln Asp Ser Lys Asp Ser
        50 55 60
Thr Tyr Ser Leu Ser Ser Thr Leu Thr Leu Ser Lys Ala Asp Tyr Glu
65 70 75 80
Lys His Lys Val Tyr Ala Cys Glu Val Thr His Gln Gly Leu Ser Ser
                                85 90 95
*1391
Pro Val Thr Lys Ser Phe Asn Arg Gly Glu Cys
                        100 105
```

| | |
|---|---|
| <210> | 27 |
| <211> | 326 |
| <212> | PRT |
| <213> | Homo sapiens |
| <400> | 27 |

```
Ala Ser Thr Lys Gly Pro Ser Val Phe Pro Leu Ala Pro Cys Ser Arg
1 5 10 15
Ser Thr Ser Glu Ser Thr Ala Ala Leu Gly Cys Leu Val Lys Asp Tyr
                        20 25 30
Phe Pro Glu Pro Val Thr Val Ser Trp Asn Ser Gly Ala Leu Thr Ser
                35 40 45
Gly Val His Thr Phe Pro Ala Val Leu Gln Ser Ser Gly Leu Tyr Ser
        50 55 60
Leu Ser Ser Val Val Thr Val Pro Ser Ser Asn Phe Gly Thr Gln Thr
65 70 75 80
Tyr Thr Cys Asn Val Asp His Lys Pro Ser Asn Thr Lys Val Asp Lys
                                85 90 95
Thr Val Glu Arg Lys Cys Cys Val Glu Cys Pro Pro Cys Pro Ala Pro
                        100 105 110
Pro Val Ala Gly Pro Ser Val Phe Leu Phe Pro Pro Lys Pro Lys Asp
                115 120 125
Thr Leu Met Ile Ser Arg Thr Pro Glu Val Thr Cys Val Val Val Asp
        130 135 140
Val Ser His Glu Asp Pro Glu Val Gln Phe Asn Trp Tyr Val Asp Gly
145 150 155 160
Val Glu Val His Asn Ala Lys Thr Lys Pro Arg Glu Glu Gln Phe Asn
                                165 170 175
Ser Thr Phe Arg Val Val Ser Val Leu Thr Val Val His Gln Asp Trp
                        180 185 190
Leu Asn Gly Lys Glu Tyr Lys Cys Lys Val Ser Asn Lys Gly Leu Pro
                195 200 205
Ala Pro Ile Glu Lys Thr Ile Ser Lys Thr Lys Gly Gln Pro Arg Glu
        210 215 220
Pro Gln Val Tyr Thr Leu Pro Pro Ser Arg Glu Glu Met Thr Lys Asn
225 230 235 240
Gln Val Ser Leu Thr Cys Leu Val Lys Gly Phe Tyr Pro Ser Asp Ile
                                245 250 255
Ser Val Glu Trp Glu Ser Asn Gly Gln Pro Glu Asn Asn Tyr Lys Thr
                        260 265 270
Thr Pro Pro Met Leu Asp Ser Asp Gly Ser Phe Phe Leu Tyr Ser Lys
                275 280 285
Leu Thr Val Asp Lys Ser Arg Trp Gln Gln Gly Asn Val Phe Ser Cys
        290 295 300
Ser Val Met His Glu Ala Leu His Asn His Tyr Thr Gln Lys Ser Leu
305 310 315 320
Ser Leu Ser Pro Gly Lys
                                325
```

| | |
|---|---|
| <210> | 28 |
| <211> | 377 |
| <212> | PRT |
| <213> | Homo sapiens |
| <400> | 28 |

```
Ala Ser Thr Lys Gly Pro Ser Val Phe Pro Leu Ala Pro Cys Ser Arg
1 5 10 15
Ser Thr Ser Gly Gly Thr Ala Ala Leu Gly Cys Leu Val Lys Asp Tyr
                        20 25 30
Phe Pro Glu Pro Val Thr Val Ser Trp Asn Ser Gly Ala Leu Thr Ser
                35 40 45
Gly Val His Thr Phe Pro Ala Val Leu Gln Ser Ser Gly Leu Tyr Ser
        50 55 60
Leu Ser Ser Val Val Thr Val Pro Ser Ser Ser Leu Gly Thr Gln Thr
65 70 75 80
Tyr Thr Cys Asn Val Asn His Lys Pro Ser Asn Thr Lys Val Asp Lys
                                85 90 95
Arg Val Glu Leu Lys Thr Pro Leu Gly Asp Thr Thr His Thr Cys Pro
                        100 105 110
Arg Cys Pro Glu Pro Lys Ser Cys Asp Thr Pro Pro Pro Cys Pro Arg
                115 120 125
Cys Pro Glu Pro Lys Ser Cys Asp Thr Pro Pro Pro Cys Pro Arg Cys
        130 135 140
Pro Glu Pro Lys Ser Cys Asp Thr Pro Pro Pro Cys Pro Arg Cys Pro
145 150 155 160
Ala Pro Glu Leu Leu Gly Gly Pro Ser Val Phe Leu Phe Pro Pro Lys
                                165 170 175
Pro Lys Asp Thr Leu Met Ile Ser Arg Thr Pro Glu Val Thr Cys Val
                        180 185 190
Val Val Asp Val Ser His Glu Asp Pro Glu Val Gln Phe Lys Trp Tyr
                195 200 205
Val Asp Gly Val Glu Val His Asn Ala Lys Thr Lys Pro Arg Glu Glu
        210 215 220
Gln Tyr Asn Ser Thr Phe Arg Val Val Ser Val Leu Thr Val Leu His
225 230 235 240
Gln Asp Trp Leu Asn Gly Lys Glu Tyr Lys Cys Lys Val Ser Asn Lys
                                245 250 255
Ala Leu Pro Ala Pro Ile Glu Lys Thr Ile Ser Lys Thr Lys Gly Gln
                        260 265 270
Pro Arg Glu Pro Gln Val Tyr Thr Leu Pro Pro Ser Arg Glu Glu Met
                275 280 285
Thr Lys Asn Gln Val Ser Leu Thr Cys Leu Val Lys Gly Phe Tyr Pro
        290 295 300
Ser Asp Ile Ala Val Glu Trp Glu Ser Ser Gly Gln Pro Glu Asn Asn
305 310 315 320
Tyr Asn Thr Thr Pro Pro Met Leu Asp Ser Asp Gly Ser Phe Phe Leu
                                325 330 335
Tyr Ser Lys Leu Thr Val Asp Lys Ser Arg Trp Gln Gln Gly Asn Ile
                        340 345 350
Phe Ser Cys Ser Val Met His Glu Ala Leu His Asn Arg Phe Thr Gln
                355 360 365
Lys Ser Leu Ser Leu Ser Pro Gly Lys
        370 375
```

| | |
|---|---|
| <210> | 29 |
| <211> | 327 |
| <212> | PRT |
| <213> | Homo sapiens |
| <400> | 29 |

```
Ala Ser Thr Lys Gly Pro Ser Val Phe Pro Leu Ala Pro Cys Ser Arg
1 5 10 15
Ser Thr Ser Glu Ser Thr Ala Ala Leu Gly Cys Leu Val Lys Asp Tyr
                        20 25 30
Phe Pro Glu Pro Val Thr Val Ser Trp Asn Ser Gly Ala Leu Thr Ser
                35 40 45
Gly Val His Thr Phe Pro Ala Val Leu Gln Ser Ser Gly Leu Tyr Ser
        50 55 60
Leu Ser Ser Val Val Thr Val Pro Ser Ser Ser Leu Gly Thr Lys Thr
65 70 75 80
Tyr Thr Cys Asn Val Asp His Lys Pro Ser Asn Thr Lys Val Asp Lys
                                85 90 95
Arg Val Glu Ser Lys Tyr Gly Pro Pro Cys Pro Ser Cys Pro Ala Pro
                        100 105 110
Glu Phe Leu Gly Gly Pro Ser Val Phe Leu Phe Pro Pro Lys Pro Lys
                115 120 125
Asp Thr Leu Met Ile Ser Arg Thr Pro Glu Val Thr Cys Val Val Val
        130 135 140
Asp Val Ser Gln Glu Asp Pro Glu Val Gln Phe Asn Trp Tyr Val Asp
145 150 155 160
Gly Val Glu Val His Asn Ala Lys Thr Lys Pro Arg Glu Glu Gln Phe
                                165 170 175
Asn Ser Thr Tyr Arg Val Val Ser Val Leu Thr Val Leu His Gln Asp
                        180 185 190
Trp Leu Asn Gly Lys Glu Tyr Lys Cys Lys Val Ser Asn Lys Gly Leu
                195 200 205
Pro Ser Ser Ile Glu Lys Thr Ile Ser Lys Ala Lys Gly Gln Pro Arg
        210 215 220
Glu Pro Gln Val Tyr Thr Leu Pro Pro Ser Gln Glu Glu Met Thr Lys
225 230 235 240
Asn Gln Val Ser Leu Thr Cys Leu Val Lys Gly Phe Tyr Pro Ser Asp
                                245 250 255
Ile Ala Val Glu Trp Glu Ser Asn Gly Gln Pro Glu Asn Asn Tyr Lys
                        260 265 270
Thr Thr Pro Pro Val Leu Asp Ser Asp Gly Ser Phe Phe Leu Tyr Ser
                275 280 285
Arg Leu Thr Val Asp Lys Ser Arg Trp Gln Glu Gly Asn Val Phe Ser
        290 295 300
Cys Ser Val Met His Glu Ala Leu His Asn His Tyr Thr Gln Lys Ser
305 310 315 320
Leu Ser Leu Ser Leu Gly Lys
                                325
```

| | |
|---|---|
| <210> | 30 |
| <211> | 332 |
| <212> | PRT |
| <213> | Artificial Sequence |
| <220> | |
| <223> | Heavy chain constant region |
| <400> | 30 |

```
Ala Ser Ala Lys Thr Thr Ala Pro Ser Val Tyr Pro Leu Ala Pro Val
1 5 10 15
Cys Gly Asp Thr Thr Gly Ser Ser Val Thr Leu Gly Cys Leu Val Lys
                        20 25 30
Gly Tyr Phe Pro Glu Pro Val Thr Leu Thr Trp Asn Ser Gly Ser Leu
                35 40 45
Ser Ser Gly Val His Thr Phe Pro Ala Val Leu Gln Ser Asp Leu Tyr
        50 55 60
Thr Leu Ser Ser Ser Val Thr Val Thr Ser Ser Thr Trp Pro Ser Gln
65 70 75 80
Ser Ile Thr Cys Asn Val Ala His Pro Ala Ser Ser Thr Lys Val Asp
                                85 90 95
Lys Lys Ile Glu Pro Arg Gly Pro Thr Ile Lys Pro Cys Pro Pro Cys
                        100 105 110
Lys Cys Pro Ala Pro Asn Leu Leu Gly Gly Pro Ser Val Phe Ile Phe
                115 120 125
Pro Pro Lys Ile Lys Asp Val Leu Met Ile Ser Leu Ser Pro Ile Val
        130 135 140
Thr Cys Val Val Val Asp Val Ser Glu Asp Asp Pro Asp Val Gln Ile
145 150 155 160
Ser Trp Phe Val Asn Asn Val Glu Val His Thr Ala Gln Thr Gln Thr
                                165 170 175
His Arg Glu Asp Tyr Asn Ser Thr Leu Arg Val Val Ser Ala Leu Pro
                        180 185 190
Ile Gln His Gln Asp Trp Met Ser Gly Lys Glu Phe Lys Cys Lys Val
                195 200 205
Asn Asn Lys Asp Leu Pro Ala Pro Ile Glu Arg Thr Ile Ser Lys Pro
        210 215 220
Lys Gly Ser Val Arg Ala Pro Gln Val Tyr Val Leu Pro Pro Pro Glu
225 230 235 240
Glu Glu Met Thr Lys Lys Gln Val Thr Leu Thr Cys Met Val Thr Asp
                                245 250 255
Phe Met Pro Glu Asp Ile Tyr Val Glu Trp Thr Asn Asn Gly Lys Thr
                        260 265 270
Glu Leu Asn Tyr Lys Asn Thr Glu Pro Val Leu Asp Ser Asp Gly Ser
                275 280 285
Tyr Phe Met Tyr Ser Lys Leu Arg Val Glu Lys Lys Asn Trp Val Glu
        290 295 300
Arg Asn Ser Tyr Ser Cys Ser Val Val His Glu Gly Leu His Asn His
305 310 315 320
His Thr Thr Lys Ser Phe Ser Arg Thr Pro Gly Lys
                                325 330
```

| | |
|---|---|
| <210> | 31 |
| <211> | 107 |
| <212> | PRT |
| <213> | Artificial Sequence |
| <220> | |
| <223> | Light chain constant region |
| <400> | 31 |

```
Arg Thr Asp Ala Ala Pro Thr Val Ser Ile Phe Pro Pro Ser Ser Glu
1 5 10 15
Gln Leu Thr Ser Gly Gly Ala Ser Val Val Cys Phe Leu Asn Asn Phe
                        20 25 30
Tyr Pro Lys Asp Ile Asn Val Lys Trp Lys Ile Asp Gly Ser Glu Arg
                35 40 45
Gln Asn Gly Val Leu Asn Ser Trp Thr Asp Gln Asp Ser Lys Asp Ser
        50 55 60
Thr Tyr Ser Met Ser Ser Thr Leu Thr Leu Thr Lys Asp Glu Tyr Glu
65 70 75 80
Arg His Asn Ser Tyr Thr Cys Glu Ala Thr His Lys Thr Ser Thr Ser
                                85 90 95
Pro Ile Val Lys Ser Phe Asn Arg Asn Glu Cys
                        100 105
```

| | |
|---|---|
| <210> | 32 |
| <211> | 245 |
| <212> | PRT |
| <213> | Artificial Sequence |
| <220> | |
| <223> | Hybrid Fc Heavy region |
| <400> | 32 |

```
Arg Asn Thr Gly Arg Gly Gly Glu Glu Lys Lys Lys Glu Lys Glu Lys
1 5 10 15
Glu Glu Gln Glu Glu Arg Glu Thr Lys Thr Pro Glu Cys Pro Ser His
                        20 25 30
Thr Gln Pro Leu Gly Val Phe Leu Phe Pro Pro Lys Pro Lys Asp Thr
                35 40 45
Leu Met Ile Ser Arg Thr Pro Glu Val Thr Cys Val Val Val Asp Val
        50 55 60
Ser Gln Glu Asp Pro Glu Val Gln Phe Asn Trp Tyr Val Asp Gly Val
65 70 75 80
Glu Val His Asn Ala Lys Thr Lys Pro Arg Glu Glu Gln Phe Asn Ser
                                85 90 95
*1748
Thr Tyr Arg Val Val Ser Val Leu Thr Val Leu His Gln Asp Trp Leu
                        100 105 110
Asn Gly Lys Glu Tyr Lys Cys Lys Val Ser Asn Lys Gly Leu Pro Ser
                115 120 125
Ser Ile Glu Lys Thr Ile Ser Lys Ala Lys Gly Gln Pro Arg Glu Pro
        130 135 140
Gln Val Tyr Thr Leu Pro Pro Ser Gln Glu Glu Met Thr Lys Asn Gln
145 150 155 160
Val Ser Leu Thr Cys Leu Val Lys Gly Phe Tyr Pro Ser Asp Ile Ala
                                165 170 175
Val Glu Trp Glu Ser Asn Gly Gln Pro Glu Asn Asn Tyr Lys Thr Thr
                        180 185 190
Pro Pro Val Leu Asp Ser Asp Gly Ser Phe Phe Leu Tyr Ser Arg Leu
                195 200 205
Thr Val Asp Lys Ser Arg Trp Gln Glu Gly Asn Val Phe Ser Cys Ser
        210 215 220
Val Met His Glu Ala Leu His Asn His Tyr Thr Gln Lys Ser Leu Ser
225 230 235 240
Leu Ser Leu Gly Lys
                                245
```

| | |
|---|---|
| <210> | 33 |
| <211> | 990 |
| <212> | DNA |
| <213> | Homo sapiens |
| <400> | 33 |

| | |
|---|---|
| gctagcacca agggcccatc ggtcttcccc ctggcgccct cctccaagag cacctctggg | 60 |
| ggcacagcgg ccctgggctg cctggtcaag gactacttcc ccgaaccggt gacggtgtcg | 120 |
| tggaactcag gggccctgac cagcggcgtg cacaccttcc cggctgtcct acagtcctca | 180 |
| ggactctact ccctcagcag cgtggtgacc gtgccctcca gcagcctggg cacccagacc | 240 |
| tacatctgca acgtgaatca caagcccagc aacaccaagg tggacaagaa agttgagccc | 300 |
| aaatcttgtg acaaaactca cacatgccca ccgtgcccag cacctgaact cctgggggga | 360 |
| ccgtcagtct tcctcttccc cccaaaaccc aaggacaccc tcatgatctc ccggacccct | 420 |
| gaggtcacat gcgtggtggt ggacgtgagc cacgaagacc ctgaggtcaa gttcaactgg | 480 |
| tacgtggacg gcgtggaggt gcataatgcc aagacaaagc cgcgggagga gcagtacaac | 540 |
| agcacgtacc gtgtggtcag cgtcctcacc gtcctgcacc aggactggct gaatggcaag | 600 |
| gagtacaagt gcaaggtctc caacaaagcc ctcccagccc ccatcgagaa aaccatctcc | 660 |
| aaagccaaag ggcagccccg agaaccacag gtgtacaccc tgcccccatc ccgggaggag | 720 |
| atgaccaaga accaggtcag cctgacctgc ctggtcaaag gcttctatcc cagcgacatc | 780 |
| gccgtggagt gggagagcaa tgggcagccg gagaacaact acaagaccac gcctcccgtg | 840 |
| ctggactccg acggctcctt cttcctctac agcaagctca ccgtggacaa gagcaggtgg | 900 |
| cagcagggga acgtcttctc atgctccgtg atgcatgagg ctctgcacaa ccactacacg | 960 |
| cagaagagcc tctccctgtc tccgggtaaa | 990 |

| | |
|---|---|
| <210> | 34 |
| <211> | 321 |
| <212> | DNA |
| <213> | Homo sapiens |
| <400> | 34 |

| | |
|---|---|
| agatctgtgg ctgcaccatc tgtcttcatc ttcccgccat ctgatgagca gttgaaatct | 60 |
| ggaactgcct ctgttgtgtg cctgctgaat aacttctatc ccagagaggc caaagtacag | 120 |
| tggaaggtgg ataacgccct ccaatcgggt aactcccagg agagtgtcac agagcaggac | 180 |
| agcaaggaca gcacctacag cctcagcagc accctgacgc tgagcaaagc agactacgag | 240 |
| aaacacaaag tctacgcctg cgaagtcacc catcagggcc tgagctcgcc cgtcacaaag | 300 |
| agcttcaaca ggggagagtg t | 321 |

| | |
|---|---|
| <210> | 35 |
| <211> | 999 |
| <212> | DNA |
| <213> | Artificial Sequence |
| <220> | |
| <223> | Heavy chain constant region |
| <400> | 35 |

| | |
|---|---|
| gctagcgcca aaacaacagc cccctccgta taccccctag cccccgtatg cggcgacacc | 60 |
| accggctcct ccgtaaccct aggctgccta gtaaaaggct acttccccga acccgtaacc | 120 |
| ctaacctgga actccggctc cctatcctcc ggcgtacaca ccttccccgc cgtactacaa | 180 |
| tccgacctat acaccctatc ctcctccgta accgtaacct cctccacctg gccctcccaa | 240 |
| tccatcacct gcaacgtagc ccaccccgcc tcctccacca aagtagacaa aaaaatcgag | 300 |
| ccaaggggcc ccaccatcaa gccttgccct ccctgcaagt gccctgcccc caacctgctg | 360 |
| ggcggcccct ccgtgttcat cttccctcca aagatcaagg acgtgctgat gatctccctg | 420 |
| agccccatcg tgacctgcgt ggtggtggac gtgtccgagg acgaccctga cgtgcagatc | 480 |
| agctggttcg tgaacaacgt ggaggtgcac accgcccaga cccagaccca cagagaggac | 540 |
| tacaactcca ccctgagggt ggtgagcgcc ctgccaatcc agcaccagga ctggatgtcc | 600 |
| ggcaaggagt tcaagtgcaa ggtgaacaac aaggacctgc cagccccaat cgagaggacc | 660 |
| atcagcaagc ctaagggctc cgtgagggcc ccccaggtgt acgtgctgcc ccctcctgag | 720 |
| gaggagatga ccaagaagca ggtgaccctg acctgcatgg tgaccgactt catgcctgag | 780 |
| gacatctacg tggagtggac caacaacggc aagaccgagc tgaactacaa gaacaccgag | 840 |
| cctgtgctgg acagcgacgg cagctacttc atgtacagca agctgagggt ggagaagaag | 900 |
| aactgggtgg agagaaactc ctactcctgc tccgtggtgc acgagggcct gcacaaccac | 960 |
| cacaccacca agagcttctc cagaaccccc gggaagtga | 999 |

| | |
|---|---|
| <210> | 36 |
| <211> | 321 |
| <212> | DNA |
| <213> | Artificial Sequence |
| <220> | |
| <223> | Light chain constant region |

| | |
|---|---|
| cgtacggatg ctgcaccaac tgtatccatc ttcccaccat ccagtgagca gttaacatct | 60 |
| ggaggtgcct cagtcgtgtg cttcttgaac aacttctacc ccaaagacat caatgtcaag | 120 |
| tggaagattg atggcagtga acgacaaaat ggcgtcctga acagttggac tgatcaggac | 180 |
| agcaaagaca gcacctacag catgagcagc accctcacgt tgaccaagga cgagtatgaa | 240 |
| cgacataaca gctatacctg tgaggccact cacaagacat caacttcacc cattgtcaag | 300 |
| agcttcaaca ggaatgagtg t | 321 |

| | |
|---|---|
| <210> | 37 |
| <211> | 457 |
| <212> | PRT |
| <213> | Artificial Sequence |
| <220> | |
| <223> | Heavy chain |
| <400> | 37 |

```
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Asn Tyr
                        20 25 30
Ala Met Ser Trp Val Arg Gln Ala Pro Gly Lys Gly Leu Glu Trp Val
                35 40 45
Ser Val Ile Ser His Gly Gly Gly Ser Thr Tyr Tyr Ala Asp Ser Val
        50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                                85 90 95
Ala Arg Val Ile Ser Asn Cys His Leu Gly Val Cys Tyr Tyr Ser Asn
                        100 105 110
Gly Met Asp Val Trp Gly Gln Gly Thr Leu Val Thr Val Ser Ser Ala
                115 120 125
Lys Thr Thr Ala Pro Ser Val Tyr Pro Leu Ala Pro Val Cys Gly Asp
        130 135 140
Thr Thr Gly Ser Ser Val Thr Leu Gly Cys Leu Val Lys Gly Tyr Phe
145 150 155 160
Pro Glu Pro Val Thr Leu Thr Trp Asn Ser Gly Ser Leu Ser Ser Gly
                                165 170 175
Val His Thr Phe Pro Ala Val Leu Gln Ser Asp Leu Tyr Thr Leu Ser
                        180 185 190
Ser Ser Val Thr Val Thr Ser Ser Thr Trp Pro Ser Gln Ser Ile Thr
                195 200 205
Cys Asn Val Ala His Pro Ala Ser Ser Thr Lys Val Asp Lys Lys Ile
        210 215 220
Glu Pro Arg Gly Pro Thr Ile Lys Pro Cys Pro Pro Cys Lys Cys Pro
225 230 235 240
Ala Pro Asn Leu Leu Gly Gly Pro Ser Val Phe Ile Phe Pro Pro Lys
                                245 250 255
Ile Lys Asp Val Leu Met Ile Ser Leu Ser Pro Ile Val Thr Cys Val
                        260 265 270
Val Val Asp Val Ser Glu Asp Asp Pro Asp Val Gln Ile Ser Trp Phe
                275 280 285
Val Asn Asn Val Glu Val His Thr Ala Gln Thr Gln Thr His Arg Glu
        290 295 300
Asp Tyr Asn Ser Thr Leu Arg Val Val Ser Ala Leu Pro Ile Gln His
305 310 315 320
Gln Asp Trp Met Ser Gly Lys Glu Phe Lys Cys Lys Val Asn Asn Lys
                                325 330 335
Asp Leu Pro Ala Pro Ile Glu Arg Thr Ile Ser Lys Pro Lys Gly Ser
                        340 345 350
Val Arg Ala Pro Gln Val Tyr Val Leu Pro Pro Pro Glu Glu Glu Met
                355 360 365
Thr Lys Lys Gln Val Thr Leu Thr Cys Met Val Thr Asp Phe Met Pro
        370 375 380
Glu Asp Ile Tyr Val Glu Trp Thr Asn Asn Gly Lys Thr Glu Leu Asn
385 390 395 400
Tyr Lys Asn Thr Glu Pro Val Leu Asp Ser Asp Gly Ser Tyr Phe Met
                                405 410 415
Tyr Ser Lys Leu Arg Val Glu Lys Lys Asn Trp Val Glu Arg Asn Ser
                        420 425 430
Tyr Ser Cys Ser Val Val His Glu Gly Leu His Asn His His Thr Thr
                435 440 445
Lys Ser Phe Ser Arg Thr Pro Gly Lys
        450 455
```

| | |
|---|---|
| <210> | 38 |
| <211> | 457 |
| <212> | PRT |
| <213> | Artificial Sequence |
| <220> | |
| <223> | Heavy chain |
| *2013 | |
| <400> | 38 |

```
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Asn Tyr
                        20 25 30
Ala Met Ser Trp Val Arg Gln Ala Pro Gly Lys Gly Leu Glu Trp Val
                35 40 45
Ser Val Ile Ser His Gly Gly Gly Ser Thr Tyr Tyr Ala Asp Ser Val
        50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                                85 90 95
Ala Arg Val Ile Ser Asn Cys His Leu Gly Val Cys Tyr Tyr Ser Asn
                        100 105 110
Gly Met Asp Val Trp Gly Gln Gly Thr Leu Val Thr Val Ser Ser Ala
                115 120 125
Ser Thr Lys Gly Pro Ser Val Phe Pro Leu Ala Pro Ser Ser Lys Ser
        130 135 140
Thr Ser Gly Gly Thr Ala Ala Leu Gly Cys Leu Val Lys Asp Tyr Phe
145 150 155 160
Pro Glu Pro Val Thr Val Ser Trp Asn Ser Gly Ala Leu Thr Ser Gly
                                165 170 175
Val His Thr Phe Pro Ala Val Leu Gln Ser Ser Gly Leu Tyr Ser Leu
                        180 185 190
Ser Ser Val Val Thr Val Pro Ser Ser Ser Leu Gly Thr Gln Thr Tyr
                195 200 205
Ile Cys Asn Val Asn His Lys Pro Ser Asn Thr Lys Val Asp Lys Lys
        210 215 220
Val Glu Pro Lys Ser Cys Asp Lys Thr His Thr Cys Pro Pro Cys Pro
225 230 235 240
Ala Pro Glu Leu Leu Gly Gly Pro Ser Val Phe Leu Phe Pro Pro Lys
                                245 250 255
Pro Lys Asp Thr Leu Met Ile Ser Arg Thr Pro Glu Val Thr Cys Val
                        260 265 270
Val Val Asp Val Ser His Glu Asp Pro Glu Val Lys Phe Asn Trp Tyr
                275 280 285
Val Asp Gly Val Glu Val His Asn Ala Lys Thr Lys Pro Arg Glu Glu
        290 295 300
Gln Tyr Asn Ser Thr Tyr Arg Val Val Ser Val Leu Thr Val Leu His
305 310 315 320
Gln Asp Trp Leu Asn Gly Lys Glu Tyr Lys Cys Lys Val Ser Asn Lys
                                325 330 335
Ala Leu Pro Ala Pro Ile Glu Lys Thr Ile Ser Lys Ala Lys Gly Gln
                        340 345 350
Pro Arg Glu Pro Gln Val Tyr Thr Leu Pro Pro Ser Arg Glu Glu Met
                355 360 365
Thr Lys Asn Gln Val Ser Leu Thr Cys Leu Val Lys Gly Phe Tyr Pro
        370 375 380
Ser Asp Ile Ala Val Glu Trp Glu Ser Asn Gly Gln Pro Glu Asn Asn
385 390 395 400
Tyr Lys Thr Thr Pro Pro Val Leu Asp Ser Asp Gly Ser Phe Phe Leu
                                405 410 415
Tyr Ser Lys Leu Thr Val Asp Lys Ser Arg Trp Gln Gln Gly Asn Val
                        420 425 430
Phe Ser Cys Ser Val Met His Glu Ala Leu His Asn His Tyr Thr Gln
                435 440 445
Lys Ser Leu Ser Leu Ser Pro Gly Lys
        450 455
```

| | |
|---|---|
| <210> | 39 |
| <211> | 218 |
| <212> | PRT |
| <213> | Artificial Sequence |
| <220> | |
| <223> | Light chain |
| *2111 | |
| <400> | 39 |

```
Gln Leu Val Leu Thr Gln Pro Pro Ser Val Ser Ala Ala Pro Gly Gln
1 5 10 15
Arg Val Ser Ile Ser Cys Ser Gly Ser Ser Ser Asn Ile Gly Asp Asn
                        20 25 30
Tyr Val Ser Trp Tyr Gln Gln Val Pro Gly Ser Ala Pro Lys Leu Leu
                35 40 45
Ile Tyr Asp Asn Asn Lys Arg Pro Ser Gly Ile Pro Asp Arg Phe Ser
        50 55 60
Ala Ser Lys Ser Gly Thr Ser Ala Thr Leu Gly Ile Thr Gly Leu Gln
65 70 75 80
Thr Gly Asp Glu Ala Asp Tyr Tyr Cys Ala Thr Trp Asp Gly Ser Leu
                                85 90 95
Ser Ala Gly Arg Val Phe Gly Gly Gly Thr Lys Leu Thr Val Leu Arg
                        100 105 110
Ala Asp Ala Ala Pro Thr Val Ser Ile Phe Pro Pro Ser Ser Glu Gln
                115 120 125
Leu Thr Ser Gly Gly Ala Ser Val Val Cys Phe Leu Asn Asn Phe Tyr
        130 135 140
Pro Lys Asp Ile Asn Val Lys Trp Lys Ile Asp Gly Ser Glu Arg Gln
145 150 155 160
Asn Gly Val Leu Asn Ser Trp Thr Asp Gln Asp Ser Lys Asp Ser Thr
                                165 170 175
Tyr Ser Met Ser Ser Thr Leu Thr Leu Thr Lys Asp Glu Tyr Glu Arg
                        180 185 190
His Asn Ser Tyr Thr Cys Glu Ala Thr His Lys Thr Ser Thr Ser Pro
                195 200 205
Ile Val Lys Ser Phe Asn Arg Asn Glu Cys
        210 215
```

| | |
|---|---|
| <210> | 40 |
| *2158<211> | 218 |
| <212> | PRT |
| <213> | Artificial Sequence |
| <220> | |
| <223> | Light chain |
| <400> | 40 |

```
Gln Leu Val Leu Thr Gln Pro Pro Ser Val Ser Ala Ala Pro Gly Gln
1 5 10 15
Arg Val Ser Ile Ser Cys Ser Gly Ser Ser Ser Asn Ile Gly Asp Asn
                        20 25 30
Tyr Val Ser Trp Tyr Gln Gln Val Pro Gly Ser Ala Pro Lys Leu Leu
                35 40 45
Ile Tyr Asp Asn Asn Lys Arg Pro Ser Gly Ile Pro Asp Arg Phe Ser
        50 55 60
Ala Ser Lys Ser Gly Thr Ser Ala Thr Leu Gly Ile Thr Gly Leu Gln
65 70 75 80
Thr Gly Asp Glu Ala Asp Tyr Tyr Cys Ala Thr Trp Asp Gly Ser Leu
                                85 90 95
Ser Ala Gly Arg Val Phe Gly Gly Gly Thr Lys Leu Thr Val Leu Arg
                        100 105 110
Thr Val Ala Ala Pro Ser Val Phe Ile Phe Pro Pro Ser Asp Glu Gln
                115 120 125
Leu Lys Ser Gly Thr Ala Ser Val Val Cys Leu Leu Asn Asn Phe Tyr
        130 135 140
Pro Arg Glu Ala Lys Val Gln Trp Lys Val Asp Asn Ala Leu Gln Ser
145 150 155 160
Gly Asn Ser Gln Glu Ser Val Thr Glu Gln Asp Ser Lys Asp Ser Thr
                                165 170 175
Tyr Ser Leu Ser Ser Thr Leu Thr Leu Ser Lys Ala Asp Tyr Glu Lys
                        180 185 190
His Lys Val Tyr Ala Cys Glu Val Thr His Gln Gly Leu Ser Ser Pro
                195 200 205
Val Thr Lys Ser Phe Asn Arg Gly Glu Cys
        210 215
```

| | |
|---|---|
| <210> | 41 |
| <211> | 1380 |
| <212> | DNA |
| <213> | Artificial Sequence |
| <220> | |
| <223> | Heavy chain |
| <400> | 41 |

| | |
|---|---|
| gaagttcagc tgcttgagtc tggcggagga ctggttcaac ctggcggaag cctgagactg | 60 |
| tcttgtgccg ccagcggctt caccttcagc aactacgcca tgagctgggt ccgacaggcc | 120 |
| cctggaaaag gccttgaatg ggtgtccgtg atctctcacg gcggaggcag cacctactac | 180 |
| gccgattctg tgaagggcag attcaccatc agccgggaca acagcaagaa caccctgtac | 240 |
| ctgcagatga acagcctgag agccgaggac accgccgtgt actattgcgc cagagtgatc | 300 |
| agcaactgcc acctgggcgt gtgctactac agcaacggca tggatgtgtg gggccagggc | 360 |
| acactggtta ccgttagttc tgctagcgcc aaaacaacag ccccctccgt atacccccta | 420 |
| gcccccgtat gcggcgacac caccggctcc tccgtaaccc taggctgcct agtaaaaggc | 480 |
| tacttccccg aacccgtaac cctaacctgg aactccggct ccctatcctc cggcgtacac | 540 |
| accttccccg ccgtactaca atccgaccta tacaccctat cctcctccgt aaccgtaacc | 600 |
| tcctccacct ggccctccca atccatcacc tgcaacgtag cccaccccgc ctcctccacc | 660 |
| aaagtagaca aaaaaatcga gccaaggggc cccaccatca agccttgccc tccctgcaag | 720 |
| tgccctgccc ccaacctgct gggcggcccc tccgtgttca tcttccctcc aaagatcaag | 780 |
| gacgtgctga tgatctccct gagccccatc gtgacctgcg tggtggtgga cgtgtccgag | 840 |
| gacgaccctg acgtgcagat cagctggttc gtgaacaacg tggaggtgca caccgcccag | 900 |
| acccagaccc acagagagga ctacaactcc accctgaggg tggtgagcgc cctgccaatc | 960 |
| cagcaccagg actggatgtc cggcaaggag ttcaagtgca aggtgaacaa caaggacctg | 1020 |
| ccagccccaa tcgagaggac catcagcaag cctaagggct ccgtgagggc cccccaggtg | 1080 |
| tacgtgctgc cccctcctga ggaggagatg accaagaagc aggtgaccct gacctgcatg | 1140 |
| gtgaccgact tcatgcctga ggacatctac gtggagtgga ccaacaacgg caagaccgag | 1200 |
| ctgaactaca agaacaccga gcctgtgctg gacagcgacg gcagctactt catgtacagc | 1260 |
| aagctgaggg tggagaagaa gaactgggtg gagagaaact cctactcctg ctccgtggtg | 1320 |
| cacgagggcc tgcacaacca ccacaccacc aagagcttct ccagaacccc cgggaagtga | 1380 |

| | |
|---|---|
| <210> | 42 |
| <211> | 1371 |
| <212> | DNA |
| <213> | Artificial Sequence |
| <220> | |
| <223> | Heavy chain |
| <400> | 42 |

| | |
|---|---|
| gaagttcagc tgcttgagtc tggcggagga ctggttcaac ctggcggaag cctgagactg | 60 |
| tcttgtgccg ccagcggctt caccttcagc aactacgcca tgagctgggt ccgacaggcc | 120 |
| cctggaaaag gccttgaatg ggtgtccgtg atctctcacg gcggaggcag cacctactac | 180 |
| gccgattctg tgaagggcag attcaccatc agccgggaca acagcaagaa caccctgtac | 240 |
| ctgcagatga acagcctgag agccgaggac accgccgtgt actattgcgc cagagtgatc | 300 |
| agcaactgcc acctgggcgt gtgctactac agcaacggca tggatgtgtg gggccagggc | 360 |
| acactggtta ccgttagttc tgctagcacc aagggcccat cggtcttccc cctggcgccc | 420 |
| tcctccaaga gcacctctgg gggcacagcg gccctgggct gcctggtcaa ggactacttc | 480 |
| cccgaaccgg tgacggtgtc gtggaactca ggggccctga ccagcggcgt gcacaccttc | 540 |
| ccggctgtcc tacagtcctc aggactctac tccctcagca gcgtggtgac cgtgccctcc | 600 |
| agcagcctgg gcacccagac ctacatctgc aacgtgaatc acaagcccag caacaccaag | 660 |
| gtggacaaga aagttgagcc caaatcttgt gacaaaactc acacatgccc accgtgccca | 720 |
| gcacctgaac tcctgggggg accgtcagtc ttcctcttcc ccccaaaacc caaggacacc | 780 |
| ctcatgatct cccggacccc tgaggtcaca tgcgtggtgg tggacgtgag ccacgaagac | 840 |
| cctgaggtca agttcaactg gtacgtggac ggcgtggagg tgcataatgc caagacaaag | 900 |
| ccgcgggagg agcagtacaa cagcacgtac cgtgtggtca gcgtcctcac cgtcctgcac | 960 |
| caggactggc tgaatggcaa ggagtacaag tgcaaggtct ccaacaaagc cctcccagcc | 1020 |
| cccatcgaga aaaccatctc caaagccaaa gggcagcccc gagaaccaca ggtgtacacc | 1080 |
| ctgcccccat cccgggagga gatgaccaag aaccaggtca gcctgacctg cctggtcaaa | 1140 |
| ggcttctatc ccagcgacat cgccgtggag tgggagagca atgggcagcc ggagaacaac | 1200 |
| tacaagacca cgcctcccgt gctggactcc gacggctcct tcttcctcta cagcaagctc | 1260 |
| accgtggaca agagcaggtg gcagcagggg aacgtcttct catgctccgt gatgcatgag | 1320 |
| gctctgcaca accactacac gcagaagagc ctctccctgt ctccgggtaa a | 1371 |

| | |
|---|---|
| <210> | 43 |
| <211> | 654 |
| <212> | DNA |
| <213> | Artificial Sequence |
| <220> | |
| <223> | Light chain |
| <400> | 43 |

| | |
|---|---|
| caactggttc tgacacagcc tccaagcgtg tcagctgccc ctggacagag agtgtccatc | 60 |
| agctgtagcg gcagcagcag caacatcggc gacaactacg tgtcctggta tcagcaggtc | 120 |
| ccaggctctg cccctaagct gctgatctac gacaacaaca agcggcccag cggcatcccc | 180 |
| gatagatttt ctgccagcaa gagcggcacc agcgccacac tgggaattac aggactgcag | 240 |
| acaggcgacg aggccgacta ctattgtgcc acatgggatg gctccctgag cgccggaaga | 300 |
| gtttttggcg gaggcaccaa gctgaccgtg cttcgtacgg atgctgcacc aactgtatcc | 360 |
| atcttcccac catccagtga gcagttaaca tctggaggtg cctcagtcgt gtgcttcttg | 420 |
| aacaacttct accccaaaga catcaatgtc aagtggaaga ttgatggcag tgaacgacaa | 480 |
| aatggcgtcc tgaacagttg gactgatcag gacagcaaag acagcaccta cagcatgagc | 540 |
| agcaccctca cgttgaccaa ggacgagtat gaacgacata acagctatac ctgtgaggcc | 600 |
| actcacaaga catcaacttc acccattgtc aagagcttca acaggaatga gtgt | 654 |

| | |
|---|---|
| <210> | 44 |
| <211> | 654 |
| <212> | DNA |
| <213> | Artificial Sequence |
| <220> | |
| <223> | Light chain |
| <400> | 44 |

| | |
|---|---|
| caactggttc tgacacagcc tccaagcgtg tcagctgccc ctggacagag agtgtccatc | 60 |
| agctgtagcg gcagcagcag caacatcggc gacaactacg tgtcctggta tcagcaggtc | 120 |
| ccaggctctg cccctaagct gctgatctac gacaacaaca agcggcccag cggcatcccc | 180 |
| gatagatttt ctgccagcaa gagcggcacc agcgccacac tgggaattac aggactgcag | 240 |
| acaggcgacg aggccgacta ctattgtgcc acatgggatg gctccctgag cgccggaaga | 300 |
| gtttttggcg gaggcaccaa gctgaccgtg cttagatctg tggctgcacc atctgtcttc | 360 |
| atcttcccgc catctgatga gcagttgaaa tctggaactg cctctgttgt gtgcctgctg | 420 |
| aataacttct atcccagaga ggccaaagta cagtggaagg tggataacgc cctccaatcg | 480 |
| ggtaactccc aggagagtgt cacagagcag gacagcaagg acagcaccta cagcctcagc | 540 |
| agcaccctga cgctgagcaa agcagactac gagaaacaca aagtctacgc ctgcgaagtc | 600 |
| acccatcagg gcctgagctc gcccgtcaca aagagcttca acaggggaga gtgt | 654 |

| | |
|---|---|
| <210> | 45 |
| <211> | 24 |
| <212> | DNA |
| <213> | Artificial Sequence |
| <220> | mouse Lrig-1 forward primer |
| <223> | |
| <400> | 45 |

| | |
|---|---|
| gacggaattc agtgaggaga acct | 24 |

| | |
|---|---|
| <210> | 46 |
| <211> | 24 |
| <212> | DNA |
| <213> | Artificial Sequence |
| <220> | mouse Lrig-1 reverse primer |
| <223> | |
| <400> | 46 |

| | |
|---|---|
| caactggtag tggcagcttg tagg | 24 |

| | |
|---|---|
| <210> | 47 |
| <211> | 24 |
| <212> | DNA |
| <213> | Artificial Sequence |
| <220> | |
| <223> | mouse Lrig-2 forward primer |
| <400> | 47 |

| | |
|---|---|
| tcacaaggaa cattgtctga acca | 24 |

| | |
|---|---|
| <210> | 48 |
| <211> | 24 |
| <212> | DNA |
| <213> | Artificial Sequence |
| <220> | |
| <223> | mouse Lrig-2 reverse primer |
| <400> | 48 |

| | |
|---|---|
| gcctgatcta acacatcctc ctca | 24 |

| | |
|---|---|
| <210> | 49 |
| <211> | 24 |
| <212> | DNA |
| <213> | Artificial Sequence |
| <220> | |
| <223> | mouse Lrig-3 forward primer |
| <400> | 49 |

| | |
|---|---|
| cagcaccttg agctgaacag aaac | 24 |

| | |
|---|---|
| <210> | 50 |
| <211> | 24 |
| <212> | DNA |
| <213> | Artificial Sequence |
| <220> | |
| <223> | mouse Lrig-3 reverse primer |
| <400> | 50 |

| | |
|---|---|
| ccagcctttg gtaatctcgg ttag | 24 |

| | |
|---|---|
| <210> | 51 |
| <211> | 24 |
| <212> | DNA |
| <213> | Artificial Sequence |
| <220> | |
| <223> | mouse FOXP3 forward primer |
| <400> | 51 |

| | |
|---|---|
| ctttcaccta tcccaccctt atcc | 24 |

| | |
|---|---|
| <210> | 52 |
| <211> | 24 |
| <212> | DNA |
| <213> | Artificial Sequence |
| <220> | |
| <223> | mouse FOXP3 reverse primer |
| <400> | 52 |

| | |
|---|---|
| attcatctac ggtccacact gctc | 24 |

| | |
|---|---|
| <210> | 53 |
| <211> | 20 |
| <212> | DNA |
| <213> | Artificial Sequence |
| <220> | |
| <223> | ACTG1 forward primer |
| <400> | 53 |

| | |
|---|---|
| ggcgtcatgg tgggcatggg | 20 |

| | |
|---|---|
| <210> | 54 |
| <211> | 20 |
| <212> | DNA |
| <213> | Artificial Sequence |
| <220> | |
| <223> | ACTG1 reverse primer |
| <400> | 54 |

| | |
|---|---|
| atggcgtggg gaagggcgta | 20 |

1-33. (canceled)

34. A binding molecule comprising:
a heavy chain variable region comprising a heavy chain CDR1 consisting of an amino acid sequence represented by SEQ ID NO: 5, a heavy chain CDR2 consisting of an amino acid sequence represented by SEQ ID NO: 6, and a heavy chain CDR3 consisting of an amino acid sequence represented by SEQ ID NO: 7; and
a light chain variable region comprising a light chain CDR1 consisting of an amino acid sequence represented by SEQ ID NO: 8, a light chain CDR2 consisting of an amino acid sequence represented by SEQ ID NO: 9, and a light chain CDR3 consisting of an amino acid sequence represented by SEQ ID NO: 10.

35. The binding molecule according to claim 34, wherein the binding molecule comprises a heavy chain variable region consisting of an amino acid sequence represented by SEQ ID NO:
11, and a light chain variable region consisting of an amino acid sequence represented by SEQ ID NO: 12.

36. The binding molecule according to claim 34, wherein the binding molecule further comprises a fragment crystallization region (Fc region) or a constant region.

37. The binding molecule according to claim 36, wherein the Fc region is the Fc region of an IgA, IgD, IgE, IgM, IgG1, IgG2, IgG3, or IgG4 antibody, or a hybrid Fc region.

38. The binding molecule according to claim 36, wherein the binding molecule further comprises a heavy chain constant region consisting of an amino acid sequence selected from the group consisting of SEQ ID NOs: 21, 23, 24, 27, 28, 29, 30 and 32.

39. The binding molecule according to claim 36, wherein the binding molecule further comprises a light chain constant region consisting of an amino acid sequence selected from the group consisting of SEQ ID NOs: 22, 25, 26, and 31.

40. The binding molecule according to claim 34, wherein the binding molecule comprises a heavy chain consisting of an amino acid sequence represented by SEQ ID NO: 37 or 38, and a light chain consisting of an amino acid sequence represented by SEQ ID NO: 39 or 40.

41. The binding molecule according to claim 34, wherein the binding molecule is an antibody or a fragment thereof.

42. The binding molecule according to claim 41, wherein the antibody is a chimeric antibody, a humanized antibody, a bivalent, a bispecific molecule, a minibody, a domain antibody, a bispecific antibody, an antibody mimetic, a unibody, a diabody, a triabody, a tetrabody, or a fragment thereof.

43. A nucleic acid molecule encoding the binding molecule of claim 34.

44. An expression vector into which the nucleic acid molecule of claim 43 is inserted.

45. A host cell line transfected with the expression vector of claim 44.

46. An antibody-drug conjugate (ADC) comprising an antibody and a drug, wherein the antibody comprises:
a heavy chain variable region comprising a heavy chain CDR1 consisting of an amino acid sequence represented by SEQ ID NO: 5, a heavy chain CDR2 consisting of an amino acid sequence represented by SEQ ID NO: 6, and a heavy chain CDR3 consisting of an amino acid sequence represented by SEQ ID NO: 7; and
a light chain variable region comprising a light chain CDR1 consisting of an amino acid sequence represented by SEQ ID NO: 8, a light chain CDR2 consisting of an amino acid sequence represented by SEQ ID NO: 9, and a light chain CDR3 consisting of an amino acid sequence represented by SEQ ID NO: 10.

47. A pharmaceutical composition for preventing or treating a brain and nervous system disease comprising, as an active ingredient, the binding molecule of claim 34.

48. The pharmaceutical composition according to claim 47, wherein the brain and nervous system disease is a neurodegenerative or neuroinflammatory disease.

49. The pharmaceutical composition according to claim 48, wherein the neurodegenerative or neuroinflammatory disease is selected from the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

50. A chimeric antigen receptor (CAR) comprising an antigen-specific binding domain, a linking domain, and a CD3 zeta (ζ) signaling domain, wherein the antigen-specific binding domain comprises:
a heavy chain variable region comprising a heavy chain CDR1 consisting of an amino acid sequence represented by SEQ ID NO: 5, a heavy chain CDR2 consisting of an amino acid sequence represented by SEQ ID NO: 6, and a heavy chain CDR3 consisting of an amino acid sequence represented by SEQ ID NO: 7; and
a light chain variable region comprising a light chain CDR1 consisting of an amino acid sequence represented by SEQ ID NO: 8, a light chain CDR2 consisting of an amino acid sequence represented by SEQ ID NO: 9, and a light chain CDR3 consisting of an amino acid sequence represented by SEQ ID NO: 10.

51. A diagnostic composition for a brain and nervous system disease comprising the binding molecule of claim 34, wherein the brain and nervous system disease is diagnosed by measuring an expression level of Lrig-1 protein present on the surface of T cells in a biological sample isolated from a target subject.

52. A method for preventing or treating a brain and nervous system disease by administering the binding molecule of claim 34 as an active ingredient into a subject.

53. A method for preventing or treating a brain and nervous system disease by administrating the antibody-drug conjugate (ADC) of claim 46 as an active ingredient into a subject.

54. A method for preventing or treating a brain and nervous system disease by administering the chimeric antigen receptor (CAR) of claim 50 as an active ingredient into a subject.
